(19) United States
(12) Patent Application Publication
(10) Pub. No.: US 2012/0171180 A1
(43) Pub. Date: Jul. 5, 2012

(54) COMPOSITIONS COMPRISING AMNION DERIVED ADHERENT CELLS AND PLATELET-RICH PLASMA

(76) Inventors: Sascha Abramson, Holland Township, NJ (US); Mohit B. Bhatia, Manalapan, NJ (US); Uri Herzberg, Bridgewater, NJ (US)

(21) Appl. No.: 13/340,550

(22) Filed: Dec. 29, 2011

Related U.S. Application Data
(60) Provisional application No. 61/428,705, filed on Dec. 30, 2010.

(51) Int. Cl. A61K 35/16; A61P 37/06; A61P 37/08; A61P 19/02; A61P 3/10; A61P 29/00; A61P 1/00; A61P 9/00; A61P 11/06

(52) U.S. Cl. 424/93.72

(57) ABSTRACT

Provided herein are methods of using amnion derived adherent cells, and populations of, and compositions comprising, such cells, in the modulation of an immune response. In various embodiments, the immune response is graft-versus-host disease, an allergy, asthma, or an immune-related disease or disorder, e.g., an autoimmune disease.

This application claims benefit of U.S. Provisional Patent Application No. 61/428,705, filed Dec. 30, 2010, which is hereby incorporated by reference in its entirety.

### 1. FIELD

Provided herein are compositions comprising amnion derived adherent cells, referred to herein as AMDACs, and platelet-rich plasma (PRP). Also provided herein are methods of treating an individual suffering from a disease or condition that would benefit from reduced inflammation, modulation of an immune response, promotion of angiogenesis, and enhanced healing, comprising administering a therapeutically effective amount of a composition comprising AMDACs cells and platelet rich plasma, as described herein, to said individual in an amount and for a time sufficient for detectable improvement of said disease or condition, e.g., a vascular condition, a non-healing or slow-healing wound, neuropathic pain, or an orthopedic defect, e.g., a spinal disc defect or arthritic joints.

### 2. BACKGROUND

Vascular conditions, non-healing or slow-healing wounds, neuropathic pain, and orthopedic defects, e.g., a spinal disc defects, among other conditions, continue to be important medical problems. There is a need for improved therapeutics for such conditions.

### 3. SUMMARY

In one aspect, provided herein are compositions comprising amnion derived adherent cells (AMDACs), or culture medium conditioned by AMDACs, and platelet-rich plasma (PRP), e.g., for use in treating a disease, disorder or medical condition in an individual.

The AMDACs useful in the methods of treatment disclosed herein may be identified by different combinations of cellular and genetic markers. In a specific embodiment, for example, said AMDACs are OCT-4⁻ as determinable by RT-PCR. In another embodiment, the AMDACs are CD49f⁺, as determinable by flow cytometry. In yet another embodiment, the AMDACs are OCT-4⁻ and CD49f⁺ as determinable by RT-PCR and flow cytometry, respectively. In still another embodiment, the AMDACs are CD49f⁺. CD105⁺, and CD200⁺ as determinable by immunolocalization, e.g., flow cytometry. In another embodiment, the AMDACs are OCT-4⁻ as determinable by RT-PCR and CD49f⁺, CD105⁺, and CD200⁺ as determinable by immunolocalization, flow cytometry. In another specific embodiment, said AMDACs are positive for VEGFR1/Flt-1 (vascular endothelial growth factor receptor 1) or CD309 (also known as VEGFR2/KDR (vascular endothelial growth factor receptor 2)), as determinable by immunolocalization, e.g., flow cytometry. In another specific embodiment, said AMDACs are CD90⁺ and CD117⁻ as determinable by flow cytometry, and HLA-G-, as determinable by RT-PCR. In another specific embodiment, said AMDACs are OCT-4⁻ and HLA-G⁻, as determinable by RT-PCR, and CD49f⁺, CD90⁺, CD105⁺, and/or CD117⁻ as determinable by immunolocalization, e.g., flow cytometry. In another specific embodiment, any of the above AMDACs are additionally one or more of CD9⁺, CD10⁺, CD44⁺, CD54⁺, CD98⁺, Tie-2⁺ (angiopoietin receptor), TEM-7⁺ (tumor endothelial marker 7), CD31⁻, CD34⁻, CD45⁻, CD133⁻, CD143⁻, CD146⁻, or CXCR4⁻ (chemokine (C-X-C motif) receptor 4) as determinable by immunolocalization, e.g., flow cytometry. In another specific embodiment, any of the above AMDACs are additionally CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and CXCR4⁻ as determinable by immunolocalization, e.g., flow cytometry.

In another specific embodiment, the AMDACs are GFAP⁺, e.g., as determinable by a short-term neural differentiation assay (see, e.g., Section 6.3.3, below). In another specific embodiment, the AMDACs are beta-tubulin III (Tuj1)⁺, e.g., as determinable by a short-term neural differentiation assay (see, e.g., Section 6.3.3, below). In another specific embodiment, the AMDACs are OCT-4⁻, GFAP⁺, and beta-tubulin III (Tuj1)⁺. In another specific embodiment, the AMDACs described herein are OCT-4⁻, CD200⁺, CD105⁺, and CD49f⁺. In another specific embodiment, the AMDACs are CD200⁺, CD105⁺, CD90⁺, and CD73⁺. In another specific embodiment, the AMDACs described herein are CD117⁻ and are not selected using an antibody to CD117. In another specific embodiment, the AMDACs are CD146⁻ and are not selected using an antibody to CD146. In another specific embodiment, the AMDACs are OCT-4⁻ and do not express CD34 following induction with VEGF as determinable by RT-PCR and/or immunolocalization (e.g., flow cytometry). In another specific embodiment, said AMDACs are OCT-4⁻, as determinable by RT-PCR, and CD49f⁺, HLA-G⁻, CD90⁺, CD105⁺, CD117⁻, and CD200⁺, as determinable by immunolocalization, e.g., flow cytometry.

In another specific embodiment, the AMDACs useful in the methods described herein are neurogenic, as determined by a short-term neural differentiation assay (see, e.g., Section 6.3.3, below). In another specific embodiment, the AMDACs useful in the methods described herein are non-chondrogenic as determined by an in vitro chondrogenic potential assay (see, e.g. Section 6.3.2, below). In another specific embodiment, the AMDACs useful in the methods described herein are non-osteogenic as determined by an osteogenic phenotype assay (see, e.g., Section 6.3.1, below). In another specific embodiment, the AMDACs described herein are non-osteogenic after being cultured for up to 6 weeks (e.g., for 2 weeks, for 4 weeks, or for 6 weeks) in DMEM at pH 7.4 (High glucose) supplemented with 100 nM Dexamethasone, 10 mM β-glycerol phosphate, 50 μM L-ascorbic acid-2-phosphate, wherein osteogenesis is assessed using von Kossa staining; alizarin red staining; or by detecting the presence of osteopontin, osteocalcin, osteonectin, and/or bone sialoprotein by, e.g., RT-PCR.

In more specific embodiments, any of the above AMDACs additionally: (a) express one or more of CD9, CD10, CD44, CD54, CD98, CD200, Tie-2, TEM-7, VEGFR1/Flt-1, or VEGFR2/KDR (CD309), as determinable by immunolocalization, e.g., flow cytometry; (b) lack expression of one or more of CD31, CD34, CD38, CD45, CD133, CD143, CD144, CD146, CD271, CXCR4, HLA-G, or VE-cadherin, as determinable by immunolocalization, e.g., flow cytometry; (c) lack expression of SOX2, as determinable by RT-PCR; (d) express mRNA for one or more of ACTA2, ADAMTS1, AMOT, ANG, ANGPT1, ANGPT2, ANGPTL1, ANGPTL2, ANGPTL4, BAI1, CD44, CD200, CEACAM1, CHGA, COL15A1, COL18A1, COL4A1, COL4A2, COL4A3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, GRN, HGF, HEY1, HSPG2, IFNB1, IL8, IL12A, ITGA4, ITGAV, ITGB3, MDK, MMP2, MYOZ2, NRP1, NRP2, PDGFB, PDGFRA, PDGFRB, PECAM1, PF4, PGK1, PROX1, PTN, SEMA3F, SERPINB5, SERPINC1, SERPINF1, TIMP2, TIMP3, TGFA, TGFB1, THBS1, THBS2, TIE1, TIE2/TEK, TNF, TNNI1, TNFSF15, VASH1, VEGF, VEGFB, VEGFC, VEGFR1/FLT1, or VEGFR2/KDR; (e) produce one or more of the proteins CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type IIB precursor, Wnt-9 protein, glial fibrillary acidic protein, astrocyte, myosin-binding protein C, or myosin heavy chain, nonmuscle type A; (f) secrete one or more of vascular endothelial growth factor (VEGF), hepatocyte growth factor (HGF), interleukin-8 (IL-8), monocyte chemotactic protein-3 (MCP-3), FGF2, Follistatin, G-CSF, EGF, ENA-78, GRO, IL-6, MCP-1, PDGF-BB, TIMP-2, uPAR, or galectin-1 into culture medium in which the AMDACs grows; (g) express one or more of micro RNAs miR-17-3p, miR-18a, miR-18b, miR-19b, miR-92, or miR-296 at a higher level than an equivalent number of bone marrow-derived mesenchymal stem cells; (h) express one or more of micro RNAs miR-20a, miR-20b, miR-221, miR-222, miR-15b, or miR-16 at a lower level than an equivalent number of bone marrow-derived mesenchymal stem cells; (i) express one or more of miRNAs miR-17-3p, miR-18a, miR-18b, miR-19b, miR-92, miR-20a, miR-20b, miR-296, miR-221, miR-222, miR-15b, and/or miR-16; or (j) express increased levels of one or more of CD202b, IL-8 or VEGF when cultured in less than about 5% O₂, compared to expression of CD202b, IL-8 or VEGF when cultured under 21% O₂. In a more specific embodiment, said AMDACs are OCT-4⁻, as determinable by RT-PCR, and CD49f, HLA-G⁻, CD90⁺, CD105⁺, CD117⁻, and CD200⁺, as determinable by immunolocalization, e.g., flow cytometry. In another specific embodiment, said AMDACs are OCT-4⁻, as determinable by RT-PCR, and CD49f⁺, HLA-G⁻, CD90⁺, CD105⁺, and CD117⁻, as determinable by immunolocalization, e.g., flow cytometry, and wherein said AMDACs additionally: (a) express CD9, CD10, CD44, CD54, CD98, CD200, Tie-2, TEM-7, VEGFR1/Flt-1, and VEGFR2/KDR (CD309), as determinable by immunolocalization, e.g., flow cytometry; (b) lack expression of CD31, CD34, CD38, CD45, CD133, CD143, CD144, CD146, CD271, CXCR4, HLA-G, and VE-cadherin, as determinable by immunolocalization, e.g., flow cytometry; (c) lack expression of SOX2, as determinable by RT-PCR; (d) express mRNA for ACTA2, ADAMTS1, AMOT, ANG, ANGPT1, ANGPT2, ANGPTL1, ANGPTL2, ANGPTL4, BAI1, CD44, CD200, CEACAM1, CHGA, COL15A1, COL18A1, COL4A1, COL4A2, COL4A3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, GRN, HGF, HEY1, HSPG2, IFNB1, IL8, IL12A, ITGA4, ITGAV, ITGB3, MDK, MMP2, MYOZ2, NRP1, NRP2, PDGFB, PDGFRA, PDGFRB, PECAM1, PF4, PGK1, PROX1, PTN, SEMA3F, SERPINB5, SERPINC1, SERPINF1, TIMP2, TIMP3, TGFA, TGFB1, THBS1, THBS2, TIE1, TIE2/TEK, TNF, TNNI1, TNFSF15, VASH1, VEGF, VEGFB, VEGFC, VEGFR1/FLT1, and VEGFR2/KDR as determinable by RT-PCR; (e) produce the proteins CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and activin receptor type IIB precursor, Wnt-9 protein, glial fibrillary acidic protein, astrocyte, myosin-binding protein C, and/or myosin heavy chain, nonmuscle type A; (t) secrete VEGF, HGF, IL-8. MCP-3, FGF2, Follistatin, G-CSF, EGF, ENA-78, GRO, IL-6, MCP-1, PDGF-BB, TIMP-2, uPAR, and Galectin-1 into culture medium in which the cell grows; (g) express micro RNAs miR-17-3p, miR-18a, miR-18b, miR-19b, miR-92, and miR-296 at a higher level than an equivalent number of bone marrow-derived mesenchymal stem cells; (h) express micro RNAs miR-20a, miR-20b, miR-221, miR-222, miR-15b, and miR-16 at a lower level than an equivalent number of bone marrow-derived mesenchymal stem cells; (i) express miRNAs miR-17-3p, miR-18a, miR-18b, miR-19b, miR-92, miR-20a, miR-20b, miR-296, miR-221, miR-222, miR-15b, and miR-16; or (j) express increased levels of CD202b, IL-8 and/or VEGF when cultured in less than about 5% O₂, compared to expression of CD202b, IL-8 and/or VEGF under 21% O₂.

In other embodiments, for example, the amnion derived adherent cells are adherent to tissue culture plastic, and are OCT-4⁻, as determinable by RT-PCR for 30 cycles, e.g., as compared to an appropriate control cell line, such as an embryonal carcinoma-derived stem cell line (e.g., NTERA-2, e.g., available from the American Type Culture Collection, ATCC Number CRL-1973). In a specific embodiment, the cells are OCT-4⁻, as determinable by RT-PCR, and VEGFR1/Flt-1⁺ (vascular endothelial growth factor receptor 1) and/or VEGFR2/KDR⁺ (vascular endothelial growth factor receptor 2, also known as kinase insert domain receptor), as determinable by immunolocalization, e.g., flow cytometry. In another specific embodiment, the cells are OCT-4⁻, as determinable by RT-PCR, and CD49f⁺ (integrin-α6⁺), as determinable by immunolocalization, e.g., flow cytometry. In a specific embodiment, said cells are OCT-4⁻, as determinable by RT-PCR, and HLA-G⁻, as determinable by RT-PCR. In another specific embodiment, said cells are OCT-4⁻, as determinable by RT-PCR, and CD90⁺, CD105⁺, or CD117⁻ as determinable by immunolocalization, e.g., flow cytometry. In a more specific embodiment, said OCT-4⁻ cells are CD90⁺, CD105⁺, and CD117⁻. In another specific embodiment, the cells are OCT-4⁻, and do not express SOX2, e.g., as determinable by RT-PCR for 30 cycles.

In another embodiment, said OCT-4⁻ cells are one or more of CD29⁺, CD73⁺, ABC-p⁺, and CD38⁻, as determinable by immunolocalization, e.g., flow cytometry.

In another specific embodiment, said OCT-4⁻ amnion derived adherent cells are additionally one or more of CD9⁺, CD10⁺, CD44⁺, CD54⁺, CD98⁺, Tie-2⁺ (angiopoietin receptor), TEM-7⁺ (tumor endothelial marker 7), CD31⁻, CD34⁻, CD45⁻, CD133⁻, CD143⁻ (angiotensin-1-converting enzyme, ACE), CD146⁻ (melanoma cell adhesion molecule), CXCR4⁻ (chemokine (C-X-C motif) receptor 4) as determinable by immunolocalization, e.g., flow cytometry. In a more specific embodiment, said amnion derived adherent cells are CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and CXCR4⁻ as determinable by immunolocalization, e.g., flow cytometry. In another more specific embodiment, the amnion derived adherent cells provided herein are OCT-4⁻, as determinable by RT-PCR; VEGFR1/Flt-1⁺ and/or VEGFR2/KDR⁺, as determinable by immunolocalization, e.g., flow cytometry: and one or more, or all, of CD31⁻, CD34⁻, CD45⁻, CD133⁻, and/or Tie-2⁻ as determinable by immunolocalization, e.g., flow cytometry. In a specific embodiment, the amnion derived adherent cells express at least 2 log less PCR-amplified mRNA for OCT-4 at, e.g., >20 cycles (e.g., 20-30 cycles), than an equivalent number of NTERA-2 cells. In another specific embodiment, said OCT-4⁻ cells are additionally VE-cadherin⁻ (CD144⁻) as determinable by immunolocalization, e.g., flow cytometry. In another specific embodiment, said OCT-4⁻ cells are additionally positive for CD105⁺ and CD200⁺ as determinable by immunolocalization, e.g., flow cytometry. In another specific embodiment, said OCT-4⁻ cells do not express CD34, e.g., as detected by immunolocalization, e.g., flow cytometry after exposure to 1 to 100 ng/mL VEGF (vascular endothelial growth factor) for 4 to 21 days.

In another embodiment, the amnion derived adherent cells are adherent to tissue culture plastic, and are OCT-4⁻ and SOX-2⁻, as determinable by RT-PCR. In yet another embodiment, said cells are CD90⁺, CD105⁺, and CD117⁻, as determinable by immunolocalization, e.g., flow cytometry. In a specific embodiments, the OCT-4⁻, SOX-2⁻ amnion derived adherent cells are additionally HLA-G⁻ or CD271⁻, as determinable by immunolocalization, e.g., flow cytometry. In a more specific embodiment, said cells are OCT-4⁻ and SOX-2⁻, as determinable by RT-PCR; and CD90⁺, CD105⁺, CD117⁻, CD271⁻ and HLA-G⁻, as determinable by immunolocalization, e.g., flow cytometry.

In another embodiment of, and in addition to, any of the above AMDACs, said cell is adherent to tissue culture plastic, and positive for CD309 (also known as VEGFR2/KDR⁺).

The amnion derived adherent cells useful in the methods of treatment disclosed herein, in another embodiment, are adherent to tissue culture plastic, are OCT-4⁻, as determinable by RT-PCR at, e.g., >20 cycles, such as 20-30 cycles, and are one or more of VEGFR2/KDR⁺, CD9⁺, CD54⁺, CD105⁺, CD200⁺, or VE-cadherin⁻, as determinable by immunolocalization, e.g., flow cytometry. In a specific embodiment, said cells are OCT-4⁻, as determinable by RT-PCR at, e.g., >20 cycles, e.g., 20-30 cycles, and VEGFR2/KDR⁺, CD9⁺, CD54⁺, CD105⁺, CD200⁺, and VE-cadherin⁻, as determinable by immunolocalization, e.g., flow cytometry. In another specific embodiment, the cells do not express CD34, e.g., as detected by immunolocalization, e.g., flow cytometry, after exposure to 1 to 100 ng/mL VEGF for 4 to 21 days.

In another embodiment, the amnion derived adherent cells are OCT-4⁻, CD49f⁺, HLA-G⁻, CD90⁺, CD105⁺, and CD117⁻. In a more specific embodiment, said cells are one or more of CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻ (melanoma cell adhesion molecule), or CXCR4⁻, as determinable by immunolocalization, e.g., flow cytometry. In a more specific embodiment, said cells are CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and CXCR4⁻ as determinable by immunolocalization, e.g., flow cytometry. In another specific embodiment, said cells are VEGFR1/Flt-1⁺ and/or VEGFR2/KDR⁺, as determinable by immunolocalization, e.g., flow cytometry; and one or more of CD31⁻, CD34⁻, CD45⁻, CD133⁻, and/or Tie-2⁺ as determinable by immunolocalization, e.g., flow cytometry. In another specific embodiment, said cell is additionally VEGFR1/Flt-1⁺, VEGFR2/KDR⁺, CD31⁻, CD34⁻, CD45⁻, CD133⁻, and Tie-2⁺ as determinable by immunolocalization, e.g., flow cytometry.

In another embodiment, the amnion derived adherent cells disclosed herein do not express mRNA for one or more of FGF4, IFNG, CXCL10, ANGPT4, ANGPTL3, FGA, LEP, PRL, PROK1, TNMD, FLT3, XLKD1, CDH5, LECT1, PLG, TERT, SOX2, NANOG, MMP-13, DLX5, or BGLAP, as determinable by RT-PCR, e.g., for 30 cycles. In another embodiment, the amnion derived adherent cells provided herein do not constitutively express one or more of invariant chain, HLA-DR-DP-DQ, CD6, or CD271, as determinable by flow cytometry, i.e., the amnion derived adherent cells do not express these markers under normal, unstimulated conditions.

In a specific embodiment, the AMDACs described herein are telomerase⁻, as measured by, e.g., RT-PCR and/or telomeric repeat amplification protocol (TRAP) assays. In another specific embodiment, the AMDACs described herein do not express mRNA for telomerase reverse transcriptase (TERT) as determinable by RT-PCR, e.g., for 30 cycles. In another specific embodiment, the AMDACs described herein are NANOG⁻, e.g., as measurable by RT-PCR. In another specific embodiment, the AMDACs described herein do not express mRNA for NANOG as determinable by RT-PCR, e.g., for 30 cycles. In a specific embodiment, the AMDACs described herein are (sex determining region Y)-box 2 (SOX2)⁻. In another specific embodiment, the AMDACs described herein do not express mRNA for SOX2 as determinable by RT-PCR, e.g., for 30 cycles.

Further provided herein is an isolated population of cells comprising amnion derived adherent cells, wherein the population of cells is therapeutically effective in the methods of treatment disclosed herein. Such populations of cells can comprise any of the amnion derived adherent cells, described by any of the combinations of markers, as disclosed herein. In specific embodiments, at least about 50%, 60%, 70%, 80%, 90%, 95%, 98% or 99% of cells in said population are such amnion derived adherent cells. In other specific embodiments, at least 25%, 35%, 45%, 50%, 60%, 75%, 85% or more of the cells in the isolated population of cells comprising amnion derived adherent cells are not OCT-4⁺.

In certain embodiments, the compositions provided herein additionally comprise a second type of stem cell. In a specific embodiment, for example, the compositions provided herein comprise isolated amnion derived adherent cells and additionally a second type of cell, e.g., stem cells or progenitor cells. In specific embodiments, the AMDACs disclosed herein comprise at least 10%, 15%, 20%, 25%, 30%, 35%, 40%, 45%, 50%, 55%, 60%, 65%, 70%, 80%, 85%, 90%, 95% or at least 98% of cells in said composition. In other specific embodiments, at least 25%, 35%, 45%, 50%, 60%, 75%, 85% or more of the cells in the composition comprising amnion derived adherent cells and a second type of cell are not OCT-4⁺. In a specific embodiment, the second type of cells are contained within or isolated from placental blood, umbilical cord blood, crude bone marrow or other tissues. In a more specific embodiment, said second type of cells are embryonic stem cells, stem cells isolated from peripheral blood, stem cells isolated from placental blood, stem cells isolated from placental perfusate, stem cells isolated from placental tissue, stem cells isolated from umbilical cord blood, umbilical cord stem cell (e.g., stem cells from umbilical cord matrix or Wharton's jelly), bone marrow-derived mesenchymal stem cells, mesenchymal stromal cells, hematopoietic stem cells or progenitor cells, e.g., CD34⁺ cells, somatic stem cell, adipose stem cells, induced pluripotent stem cells, or the like. In another more specific embodiment, said second type of cells comprise at least 10%, 15%, 20%, 25%, 30%, 35%, 40%, 45%, or 50% of cells in said population.

In another specific embodiment, any of the above AMDACs, or second type of cells, are, or have been, proliferated in culture. In another specific embodiment, any of the above cells are from a culture of such cells that has been passaged at least 1, 2, 3, 4, 5, 6, 7, 8, 9, 10, 11, 12, 13, 14, 15, 16, 17, 18, 19, or 20 times, or more. In another specific embodiment, any of the above cells are from a culture of such cells that has doubled at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or at least 50 times, or more.

Said composition comprising AMDACs is formulated to be administered to said individual by injection, e.g., local injection.

In some embodiments, the platelet rich plasma of the compositions provided herein is autologous platelet rich plasma. In some embodiments, the platelet rich plasma is derived from placental perfusate. In other embodiments, the [platelet-rich plasma is obtained or derived from a suitable donor.

In some embodiments, the volume to volume ratio of AMDACs to platelet rich plasma in the composition is between about 10:1 and 1:10. In some embodiments, the volume to volume ratio of AMDACs to platelet rich plasma in the composition is about 1:1. In some embodiments, the ratio of the number of AMDACs to the number of platelets in the platelet rich plasma is between about 100:1 and 1:100. In some embodiments, the ratio of the number of AMDACs to the number of platelets in the platelet rich plasma is about 1:1.

In another aspect, provided herein is a method of transplantation comprising administering to an individual, e.g., injecting an individual with, a composition comprising AMDACs and platelet rich plasma, wherein said transplantation results in prolonged localization of said AMDACs at the site of injection, or region, relative to injection of AMDACs not combined with platelet rich plasma.

In some embodiments, the platelet rich plasma is autologous platelet rich plasma. In some embodiments, the platelet rich plasma is derived from placental perfusate.

In some embodiments, the AMDACs and platelet rich plasma are combined to form said composition ex vivo prior to said injecting the individual. In some embodiments, the platelet rich plasma is injected into the individual in a first step, and the AMDACs are injected into or near the site of platelet rich plasma injection in a second step, and the composition is formed in vivo.

In some embodiments of the methods of transplantation provided herein, the volume to volume ratio of AMDACs to platelet rich plasma in the composition is between about 10:1 and 1:10. In some embodiments, the volume to volume ratio of AMDACs to platelet rich plasma in the composition is about 1:1. In some embodiments, the ratio of the number of AMDACs to the number of platelets in the platelet rich plasma is between about 100:1 and 1:100. In some embodiments, the ratio of the number of AMDACs to the number of platelets in the platelet rich plasma is about 1:1.

In another aspect, provided herein is a method of treating an individual having or at risk of developing critical limb ischemia, comprising administering to the individual a therapeutically effective amount of a composition comprising AMDACs and platelet rich plasma. In another aspect, provided herein is a method of treating an individual having leg ulcer, comprising contacting the leg ulcer with a therapeutically effective amount of a composition comprising AMDACs and platelet rich plasma. In some embodiments, the leg ulcer is a venous leg ulcer, arterial leg ulcer, diabetic leg ulcer, decubitus ulcer, or split thickness skin grafted ulcer.

In another aspect, provided herein is a method of treating an individual having degenerative disc disorder, comprising administering to the individual a therapeutically effective amount of a composition comprising AMDACs and platelet rich plasma.

In another aspect, provided herein is a method of treating an individual having herniated disc, comprising contacting the herniated disc with a therapeutically effective amount of a composition comprising AMDACs and platelet rich plasma.

In another aspect, provided herein is a method of treating an individual having neuropathic pain, comprising administering to the individual a therapeutically effective amount of a composition comprising AMDACs and platelet rich plasma.

The isolated amnion derived adherent cells and cell populations provided herein are not the isolated placental stem cells or cell populations described, e.g., in U.S. Pat. No. 7,255,879 or U.S. Patent Application Publication No. 2007/0275362. The isolated amnion derived adherent cells provided herein are also not endothelial progenitor cells, amniotic epithelial cells, trophoblasts, cytotrophoblasts, embryonic germ cells, embryonic stem cells, cells obtained from the inner cell mass of an embryo, or cells obtained from the gonadal ridge of an embryo.

### 3.1 DEFINITIONS

As used herein, the term “about,” when referring to a stated numeric value, indicates a value within plus or minus 10% of the stated numeric value.

As used herein, the term “amount,” when referring to the AMDACs described herein, means a particular number of AMDACs.

As used herein, the term “stem cell” defines a cell that retains at least one attribute of a stem cell, e.g., a marker or gene expression profile associated with one or more types of stem cells; the ability to replicate at least 10-40 times in culture; multipotency, e.g., the ability to differentiate, either in vitro, in vivo or both, into cells of one or more of the three germ layers; the lack of adult (i.e., differentiated) cell characteristics, or the like.

As used herein, the term “derived” means isolated from or otherwise purified. For example, amnion derived adherent cells can be isolated from amnion, or cultured from cells isolated from amnion. The term “derived” encompasses cells that are cultured from cells isolated directly from a tissue, e.g., the amnion, and cells cultured or expanded from primary isolates.

As used herein, “immunolocalization” means the detection of a compound. e.g., a cellular marker, using an immune protein, e.g., an antibody or fragment thereof in, for example, flow cytometry, fluorescence-activated cell sorting, magnetic cell sorting, in situ hybridization, immunohistochemistry, or the like.

As used herein, stem cells, e.g., AMDACs are “isolated” if at least 50%, 60%, 70%, 80%, 90%, 95%, or at least 99% of other cells, with which the stem cells are naturally associated, are removed from the stem cells, e.g., during collection and/or culture of the stem cells.

As used herein, the term “isolated population of cells” means a population of cells that is substantially separated from other cells of the tissue, e.g., amnion, from which the population of cells is obtained or derived. In some embodiments, a population of, e.g., stem cells is “isolated” if at least 50%, 60%, 70%, 80%, 90%, 95%, or at least 99% of the cells with which the population of stem cells are naturally associated are removed from the population of stem cells, e.g., during collection and/or culture of the population of stem cells.

As used herein, AMDACs are “positive” for a particular marker when that marker is detectable above background, e.g., by immunolocalization, e.g., by flow cytometry; or by RT-PCR, etc. For example, a cell or cell population is described as positive for, e.g., CD105 if CD105 is detectable on the cell, or in the cell population, in an amount detectably greater than background (in comparison to, e.g., an isotype control), or an experimental negative control for any given assay). In the context of, e.g., antibody-mediated detection, “positive,” as an indication a particular cell surface marker is present, means that the marker is detectable using an antibody, e.g., a fluorescently-labeled antibody, specific for that marker; “positive” also means that a cell or population of cells displays that marker in a amount that produces a signal, e.g., in a cytometer, ELISA, or the like, that is detectably above background. For example, a cell is “CD 105⁺” where the cell is detectably labeled with an antibody specific to CD105, and the signal from the antibody is detectably higher than a control (e.g., background). Conversely, “negative” in the same context means that the cell surface marker is not detectable using an antibody specific for that marker compared to background. For example, a cell or population of cells is “CD34⁻” where the cell or population of cells is not detectably labeled with an antibody specific to CD34. Unless otherwise noted herein, cluster of differentiation (“CD”) markers are detected using antibodies. For example, OCT-4 can be determined to be present, and a cell is OCT-4⁺, if mRNA for OCT-4 is detectable using RT-PCR, e.g., for 30 cycles. A cell is also positive for a marker when that marker can be used to distinguish the cell from at least one other cell type, or can be used to select or isolate the cell when present or expressed by the cell.

As used herein, “immunomodulation” and “immunomodulatory” mean causing, or having the capacity to cause, a detectable change in an immune response, and the ability to cause a detectable change in an immune response either systemically or locally.

As used herein, “immunosuppression” and “immunosuppressive” mean causing, or having the capacity to cause, a detectable reduction in an immune response, and the ability to cause a detectable suppression of an immune response either systemically or locally.

### 4. BRIEF DESCRIPTION OF THE FIGURES

FIG. 1 shows expression of stem cell-related genes by amnion derived adherent cells and NTERA-2 cells.

FIG. 2 shows the expression of TEM-7 on the cell surface of amnion derived adherent cells (AMDACs).

FIGS. 3A-3D show the secretion of selected angiogenic proteins by amnion derived adherent cells. FIG. 3A: Secretion by passage six AMDACs (n=3) of tissue inhibitor of metalloprotease-1 (TIMP-1), TIMP-2, thrombopoietin, vascular endothelial growth factor (VEGF), and VEGF-D. FIG. 3B: Secretion by passage six AMDACs (n=3) of angiogenin, epidermal growth factor (EGF), epithelial neutrophil-activating peptide 78 (ENA-78), basic fibroblast growth factor (bFGF), and growth-regulated oncogene alpha (GRO). FIG. 3C: Secretion by passage six AMDACs (n=3) of interferon gamma (IFN-gamma), insulin-like growth factor-1 (IGF-1), interleukin-6 (IL-6), IL-8, and leptin. FIG. 3D: Secretion by passage six AMDACs (n=3) of monocyte chemotactic protein-1 (MCP-1), platelet-derived growth factor (PDGF)-BB, placental growth factor (PlGF), ranter, and transforming growth factor-beta (TGF-beta).

FIG. 4 demonstrates the ability of AMDACs to inhibit T cell proliferation in vitro. NHDF: neonatal human dermal fibroblasts. Bars to left for AMDAC, NHDF: CD4+ T cell suppression compared to absence of AMDACs or NHDFs. Bars to right for AMDAC, NHDF: CD8+ T cell suppression compared to absence of AMDACs or NHDFs. Y axis: percent suppression attributable to AMDACs or NHDFs as compared to T cell proliferation in the absence of AMDACs or NHDFs.

FIG. 5 demonstrates that media conditioned by AMDACs induces suppression of TNF-alpha production by T cells. Y axis: percent suppression of production of TNF-α by bulk T cells in the presence of AMDACs or NHDFs as compared to production of TNF-α in the absence of AMDACs or NHDFs.

FIG. 6 shows suppression by AMDACs of Th1 T cells. Pan T base: percent of Th1 T cells in the absence of AMDACs. 100K, 75K, 50K, 25K: percent Th1 T cells in the presence of 100,000, 75,000, 50,000, and 25,000 AMDACs, respectively.

FIG. 7 shows suppression by AMDACs of Th17 T cells in a dose-dependent manner. 100K, 80K, 60K, 40K: percent Th17 T cells (in the absence of AMDACs) remaining after coculture with 100,000, 80,000, 60,000, and 40,000 AMDACs, respectively.

FIG. 8 shows increase of FoxP3 Treg cells by AMDACs. Baseline: percent of FoxP3 Treg cells in total T cells in the absence of AMDACs. 100K, 75K, 50K, 25K: percent FoxP3 Treg cells in the presence of 100,000, 75,000, 50,000, and 25,000 AMDACs, respectively.

FIGS. 9A-9C depict flow cytometry results of DC populations as assessed by CD86 and HLA-DR expression. All: SSC: side scatter gate. Cell type: dendritic cells (DC) alone, or DC+AMDACs. LPS+IFN-γ: cells stimulated (+) or not stimulated (−) with bacterial lipopolysaccharide and interferon gamma. FIG. 9A: DC labeled with anti-CD86-phycoerythrin (PE). FIG. 9B: DC labeled with anti-HLA-DR-PerCP Cy5.5. FIG. 9C: DC labeled with anti-IL-12-PE (Y-axis) and anti-CD11c-FITC.

FIG. 10 depicts suppression of production of tumor necrosis factor-alpha (TNF-α) and interleukin-12 (IL-12 by bacterial lipopolysaccharide (LPS)-stimulated dendritic cells (DCs). For each condition (IL-12 or TNF-α production), the left column is the production of the cytokine by DCs in the presence of LPS and interferon-gamma (IFN-γ), and the right column is the production of the cytokine by DCs in the presence of LPS, IFN-γ, and AMDACs. “□−” indicates condition in which DCs were not stimulated with either LPS or IFN-γ. Numbers to the right of each condition indicate the number of picograms of IL-12 or TNF-α produced by DC in each condition.

FIG. 11 depicts AMDAC-mediated suppression of natural killer (NK) cell proliferation. X axis: number of days of culture of NK cell precursors with (left bars) or without (right bars) AMDACs. Y axis: number of NK cells at each day of culture indicated.

FIG. 12 depicts AMDAC suppression of NK cell cytotoxicity. X axis: number of AMDACs per well in a coculture with NK cells and K562 cells (a human immortalized myelogenous leukaemia cell line) as targets. Y axis: Percent NK cytotoxicity, calculated as (1−total number of K562 cells in the sample÷total K562 cells in a control containing no NK cells)×100.

### 5. DETAILED DESCRIPTION

### 5.1 AMDACS and Platelet-Rich Plasma

Provided herein are compositions comprising AMDACs combined with platelet rich plasma, wherein administration of the compositions to an individual in need thereof results in, e.g., prolonged localization of the AMDACs at the site of injection or implantation, relative to administration of AMDACs not combined with platelet rich plasma. In certain embodiments, the AMDACs are human. In other embodiments, the platelet rich plasma is human, e.g., is obtained from or derived from a human source. In other embodiments, both the AMDACs and PRP are human.

In various embodiments, the volume to volume ratio of AMDACs (e.g., AMDACs in suspension) to platelet rich plasma can be between about 10:1 and 1:10. In some embodiments, the volume to volume ratio of AMDACs to platelet rich plasma is about 10:1, 9.5:1, 9:1, 8.5:1, 8:1, 7.5:1, 7:1, 6.5:1, 6:1, 5.5:1, 5:1, 4.5:1, 4:1, 3.5:1, 3:1, 2.5:1, 2:1, 1.5:1, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1.9.5, or 1:10. In particular embodiments, the volume to volume ratio of AMDACs to platelet rich plasma is about 1:1. In some embodiments, the ratio of the number of AMDACs to the number of platelets in the platelet rich plasma can be between about 100:1 and 1:100. In some embodiments, the volume to volume ratio of AMDACs to platelet rich plasma is about 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10 1:15, 1:20, 1:25, 1:30, 1:35, 1:40, 1:45, 1:50, 1:55, 1:60, 1:65, 1:70, 1:75, 1:80, 1:85, 1:90, 1:95, or 1:100. In particular embodiments, the ratio of the number of AMDACs to the number of platelets in the platelet rich plasma is about 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10 1:15, 1:20, 1:25, 1:30, 1:35, 1:40, 1:45 1:50, 1:55, 1:60, 1:65, 1:70, 1:75, 1:80, 1:85, 1:90, 1:95, or 1:100.

The compositions comprising AMDACs and platelet rich plasma provided herein can comprise a therapeutically-effective amount of AMDACs, platelets, e.g., platelet rich plasma, or both. The combination compositions can comprise at least 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, or 1×10¹¹ AMDACs, platelets in platelet rich plasma, or both, or no more than 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, or 1×10¹¹ AMDACs, platelets in platelet rich plasma, or both.

### 5.2 Platelet-Rich Plasma

The compositions and methods provided herein use AMDACs in combination with platelet rich plasma (PRP). In some embodiments, PRP useful in the combination compositions and methods provided herein comprises platelet cells at a concentration of at least 1.1-fold greater than the concentration of platelets in whole blood, e.g., unprocessed whole blood. In some embodiments, the PRP comprises platelet cells at a concentration of about 1.1-fold to about 10-fold greater than the concentration of platelets in whole blood. In some embodiments, the PRP comprises platelet cells at a concentration of about 1.5, 2.0, 2.5, 3.0, 3.5, 4, 4.5, 5, 5.5, 6, 6.5, 7, 7.5, 8, 8.5, 9, 9.5, 10-fold, or more than 10-fold greater than the concentration of platelets in whole blood.

Generally, a microliter of whole blood comprises between 140,000 and 500,000 platelets. In some embodiments, the platelet concentration in the PRP useful in the combination compositions and methods provided herein is between about 150,000 and about 2,000,000 platelets per microliter. In some embodiments, the platelet concentration in the PRP is about 150,000, 200,000, 300,000, 400,000, 500,000, 600,000, 700,000, 800,000, 900,000, 1,000,000, 1,100,000, 1,100,000, 1,200,000, 1,300,000, 1,400,000, 1,500,000, 1,600,000, 1,700,000, 1,800,000, 1,900,000, or 2,000,000 platelets per microliter. In some embodiments, the platelet concentration in the PRP is about 2,500,000 to about 5,000,000, or about 5,000,000 to about 7,000,000 platelets per microliter.

The combination compositions provided herein may comprise PRP derived from a human or animal source of whole blood. The PRP may be prepared from an autologous source, an allogeneic source, a single source, or a pooled source of platelets and/or plasma, e.g., platelets harvested from placental perfusate. The PRP can be isolated from whole blood or portions of whole blood using a variety of techniques comprising, for example, centrifugation, gravity filtration, and/or direct cell sorting.

PRP can be, e.g., prepared from a donor who has not been previously treated with a thrombolytic agent, such as heparin, tPA, or aspirin. In some embodiments, the donor has not received a thrombolytic agent for at least 2 hours, 1 day, 2 weeks, or 1 month prior to withdrawing the blood for extraction of the PRP.

To derive PRP from donor blood, whole blood may be collected from the donor, for example, using a blood collection syringe. The amount of blood collected may depend on a number of factors, including, for example, the amount of PRP desired, the health of the donor, the severity or location of the tissue damage in the individual to be treated, the availability of prepared PRP, or any suitable combination of factors.

Any suitable amount of blood may be collected. For example, about 30 to 60 ml of whole blood may be drawn. In an exemplary embodiment, about 11 ml of blood may be withdrawn into a syringe that contains about 5 ml of an anticoagulant, such as acid-citrate-phosphate or citrate-phosphate-dextrose solution. The syringe may be attached to an apheresis needle, and primed with the anticoagulant. Blood may be drawn from the donor using standard aseptic practice. In some embodiments, a local anesthetic such as anbesol, benzocaine, lidocaine, procaine, bupivicaine, or any appropriate anesthetic known in the art may be used to anesthetize the insertion area.

5.2.1 Methods of Obtaining Platelet Rich Plasma

Isolation of platelets from whole blood depends upon the density difference between platelets and red blood cells. The platelets and white blood cells are concentrated in the layer (i.e., the “buffy coat”) between the platelet depleted plasma (top layer) and red blood cells (bottom layer). For example, a bottom buoy and a top buoy may be used to trap the platelet-rich layer between the upper and lower phase. This platelet-rich layer may then be withdrawn using a syringe or pipette. Generally, at least 60% or at least 80% of the available platelets within the blood sample can be captured. These platelets may be resuspended in a volume that may be about 3% to about 20% or about 5% to about 10% of the sample volume. PRP may be isolated from whole blood by any method known in the art. For example, the PRP may be prepared from whole blood using a centrifuge. In a particular embodiment, whole blood is spun at 150-1350×g for 6 minutes at room temperature.

In another embodiment, whole blood can be centrifuged using a gravitational platelet system, such as the Cell Factor Technologies GPS SYSTEM™ centrifuge. The blood-filled syringe may be slowly transferred to a disposable separation tube which may be loaded into a port on the GPS centrifuge. The sample may be capped and placed into the centrifuge. The centrifuge may be counterbalanced with a tube comprising sterile saline, placed into the opposite side of the centrifuge. Alternatively, if two samples are prepared, two GPS disposable tubes may be filled with equal amounts of blood and citrate dextrose. The samples may then be spun to separate platelets from blood and plasma. The samples may be spun at about 2000 rpm to about 5000 rpm for about 5 minutes to about 30 minutes. For example, centrifugation may be performed at 3200 rpm for extraction from a side of the separation tube and then isolated platelets may be suspended in about 3 cc to about 5 cc of plasma by agitation. The PRP may then be extracted from a side port using, for example, a 10 cc syringe. If about 55 cc of blood is collected from a patient, about 5 cc of PRP may be obtained.

The PRP may be buffered using an alkaline buffering agent to a physiological pH. The buffering agent may be a biocompatible buffer such as HEPES, TRIS, monobasic phosphate, monobasic bicarbonate, or any suitable combination thereof capable of adjusting the PRP to physiological pH between about 6.5 and about 8.0. In certain embodiments, the physiological pH is adjusted to about pH 7.3 to about pH 7.5, more specifically, about pH 7.4. In certain embodiments, the buffering agent is an 8.4% sodium bicarbonate solution. In this embodiment, for each cc of PRP isolated from whole blood, 0.05 cc of 8.4% sodium bicarbonate may be added. In some embodiments, the syringe may be gently shaken to mix the PRP and bicarbonate.

Platelet counts in the PRP can be counted and recorded, and the PRP can be resuspended for a precise number of wells in a compatible vehicle or in the donor's own plasma prior to combining with AMDACs according to the methods described herein.

In some embodiments of the compositions and methods provided herein, the composition comprises AMDACs and PRP derived from placental perfusate. An exemplary method for isolating PRP from placental perfusate is as follows. Following exsanguination of the umbilical cord and placenta, the placenta is placed in a sterile, insulated container at room temperature and delivered to the laboratory within 4 hours of birth. The placenta is discarded if, on inspection, there is evidence of physical damage such as fragmentation of the organ or avulsion of umbilical vessels. The placenta is maintained at room temperature (23°+/−2° C.) or refrigerated (4° C.) in sterile containers for 2 to 20 hours. Periodically, the placenta is immersed and washed in sterile saline at 25°+/−3° C. to remove any visible surface blood or debris. The umbilical cod is transected approximately 5 cm from its insertion into the placenta and the umbilical vessels are cannulated with Teflon or polypropylene catheters connected to a sterile fluid path allowing bidirectional perfusion of the placenta and recovery of the effluent fluid.

The cannula can be, e.g., flushed with IMDM serum-free medium (GibcoBRL, NY) containing 2 U/ml heparin (Elkins-Sinn, N.J.). In one embodiment, perfusion of the placenta continues at a rate of 50 mL per minute until approximately 300-750 mL of perfusate is collected. During the course of the procedure, the placenta may be gently massaged to aid in the perfusion process and assist in the recovery of cellular material. Effluent fluid is collected from the perfusion circuit by both gravity drainage and aspiration through the arterial cannula.

The perfusion and collection procedures may be repeated once or twice until the number of recovered nucleated cells falls below 100 μL. The perfusates are pooled and subjected to light centrifugation to isolate platelets. Platelets can be can be resuspended for a precise number of wells in a compatible vehicle or in the donor's own plasma prior to combining with AMDACs according to the methods described herein.

### 5.3 Methods of Transplanting Compositions Comprising AMDACS and Platelet Rich Plasma

In some embodiments, an individual is contacted with a combination composition comprising AMDACs and platelet rich plasma as provided herein. In a specific embodiment, said contacting is the introduction, e.g., transplantation, of said combination composition into said individual. Thus, the method of combining AMDACs with platelet rich plasma may be performed as a first step in a procedure for introducing the combination composition into any individual needing stem cells, e.g., AMDACs. Such a procedure can comprise use of pharmaceutical compositions comprising the combination compositions, as described above. Alternatively, each component of the combination composition can be introduced, e.g., transplanted into said individual serially. For example, platelet rich plasma may be administered to the individual in a first step, near the area where the pathogenesis is present, to form a stable hydrogel in vivo. In a second step, AMDACs may be administered, e.g., injected into the formed hydrogel.

In a specific embodiment, AMDACs are combined with platelet rich plasma prior to administration to an individual in need thereof in a ratio (e.g., by volume or number of cells) that results in prolonged localization of the AMDACs at the site of injection or implantation, relative to administration of AMDACs not combined with platelet rich plasma. In another specific embodiment. AMDACs are combined with platelet rich plasma during, or simultaneously with, administration to an individual in need thereof, in an optimum ratio, that results in prolonged localization of the AMDACs at a site of injection or implantation, relative to administration of AMDACs not combined with platelet rich plasma. In another specific embodiment, AMDACs and platelet rich plasma are administered sequentially to an individual in need thereof to a final optimum ratio. In one embodiment, the AMDACs are administered first and the platelet rich plasma is administered second. In another embodiment, the platelet rich plasma is administered first and the AMDACs are administered second.

In a specific embodiment, said composition comprising AMDACs and platelet rich plasma is contained within one bag or container. In another embodiment, provided herein is the use in transplantation of AMDACs, and platelet rich plasma, that are contained within separate bags or containers. In certain embodiments, AMDACs and platelet rich plasma contained in two separate bags may be mixed prior, in particular immediately prior, to or at the time of administration to an individual in need thereof.

The combining, i.e., mixing of AMDACs with platelet rich plasma to obtain the combination compositions provided herein is generally performed gently so as to not activate the platelets within the PRP.

In particular embodiments, the AMDACs and platelet rich plasma are provided in separate chambers of a 2-chamber syringe and reconstituted in the syringe prior to administration, e.g., injection into the individual.

Compositions comprising AMDACs and platelet rich plasma may be mixed, prior to transplantation, by any medically-acceptable means. In one embodiment, the two components are physically mixed. In another embodiment of the method, the AMDACs and the platelet rich plasma are mixed immediately prior to (i.e., within 1, 2, 3, 4, 5, 7, 10, 20 or 30 minutes of) administration to said individual. In another embodiment, the AMDACs and the platelet rich plasma are mixed at a point in time more than five minutes prior to administration to said individual. In another embodiment of the method, the AMDACs and/or platelet rich plasma are cryopreserved and thawed prior to administration to said individual. In another embodiment, said AMDACs and platelet rich plasma are mixed to form a composition at a point in time more than 1, 2, 3, 4, 5, 6, 7, 8, 9, 10, 11, 12, 13, 14, 15, 16, 17, 18, 19, 20, 21, 22, 23, or 24 hours prior to administration to said individual, wherein either or both of the AMDACs and platelet rich plasma have been cryopreserved and thawed prior to said administration. In another embodiment, the composition comprising AMDACs and AMDACs may be administered to an individual more than once.

In some embodiments, the platelet rich plasma component of the composition, when administered separately from the AMDACs component, can be administered as a liquid, a solid, a semi-solid (e.g., a gel), or a combination thereof. In such embodiments, when the platelet rich plasma is delivered as a liquid, it may comprise a solution, an emulsion, a suspension, or the like.

In some embodiments, a platelet rich plasma semi-solid or gel may be prepared by adding an agent to the platelet rich plasma, alone or combined with AMDACs, e.g., to better preserve the position of the AMDACs once the combination composition is delivered to the target tissue, For example, the platelet rich plasma, alone or in combination with AMDACs, may include collagen, cyanoacrylate, adhesives that cure upon injection into tissue, liquids that solidify or gel after injection into tissue, suture material, agar, gelatin, light-activated dental composite, other dental composites, silk-elastin polymers, MATRIGEL™, gelatinous protein mixture (e.g., from BD Biosciences), hydrogels and/or other suitable biopolymers. In certain other embodiments, a clotting agent (e.g., thrombin and/or calcium) may be added to the PRP above or combined with AMDACs. Alternatively, the clotting agent may be delivered to a target tissue before or after platelet rich plasma, alone or in combination with AMDACs, has been delivered to the target tissue to cause the platelet rich plasma to gel. In other embodiments, no clotting agents are added to the platelet rich plasma or to the combination composition comprising platelet rich plasma and AMDACs. In particular embodiments, the composition comprising AMDACs combined with platelet rich plasma, provided herein, does not comprise, and does not require, a clotting agent (e.g., thrombin and/or calcium) to effect prolonged localization of the AMDACs at the site of injection or implantation, relative to AMDACs not administered in combination with platelet rich plasma. For example, platelet rich plasma, alone or in combination with AMDACs, may harden or gel in response to one or more environmental or chemical factors such as temperature, pH, proteins, etc., without the addition of a clotting agent.

In another embodiment, the AMDACs contained within the composition are preconditioned prior to transplantation. In a various embodiments, preconditioning comprises storing the cells in a gas-permeable container generally for a period of time at about −5° C. to about 23° C., about 0° C. to about 10° C., or about 4° C. to about 5° C. The cells may be stored between 18 hours and 21 days, between 48 hours and 10 days, preferably between 3-5 days. The cells may be cryopreserved prior to preconditioning or, may be preconditioned immediately prior to administration.

In some embodiments, the AMDACs may be differentiated prior to introduction of the combination composition to an individual in need of stem cells, e.g., AMDACs. The combination of differentiated AMDACs and platelet rich plasma is encompassed within the phrase “combination composition.” In certain embodiments, the method of transplantation of a combination composition provided herein comprises (a) induction of differentiation of AMDACs, (b) mixing the AMDACs with platelet rich plasma to form a combination composition, and (c) administration of the combination composition to an individual in need thereof. In certain other embodiments, the method of transplantation of a combination composition provided herein comprises (a) mixing the AMDACs with platelet rich plasma to form a combination composition, (b) induction of differentiation of AMDACs, and (c) administration of the combination composition to an individual in need thereof.

The compositions provided herein, comprising AMDACs and platelet rich plasma, or each component of the composition, may be transplanted into an individual in any pharmaceutically or medically acceptable manner, including by surgical implantation or injection, e.g., intravenous injection, intraarterial injection, intra-articular injection, intramuscular injection, intraperitoneal injection, intraocular injection, direct injection into a particular tissue. The site of delivery of the composition is typically at or near the site of pathogenesis, e.g., tissue damage. The site of tissue damage can be determined by well-established methods including medical imaging, patient feedback, or a combination thereof. The particular imaging method used may be determined based on the tissue type. Commonly used imaging methods include, but are not limited to MRI, X-ray, CT scan, Positron Emission tomography (PET), Single Photon Emission Computed Tomography (SPECT), Electrical Impedance Tomography (EIT), Electrical Source Imaging (ESI), Magnetic Source Imaging (MSI), laser optical imaging and ultrasound techniques. The patient may also assist in locating the site of tissue injury or damage by pointing out areas of particular pain and/or discomfort. The PRP composition may be delivered minimally invasively and/or surgically. For example, to deliver a PRP composition to ischemic tissue, a physician may use one of a variety of access techniques, including but not limited to, surgical (e.g., sternotomy, thoracotomy, mini-thoracotomy, sub-xiphoidal) approaches, endoscopic approaches (e.g., intercostal and transxiphoidal) and percutaneous (e.g., transvascular) approaches.

The composition may comprise, or be suspended in, any pharmaceutically-acceptable carrier. The combination composition may be carried, stored, or transported in any pharmaceutically or medically acceptable container, for example, a blood bag, transfer bag, plastic tube or vial.

After transplantation, situation, placement or engraftment in a human recipient may be assessed using, e.g., nucleic acid or protein detection or analytical methods. For example, the polymerase chain reaction (PCR), STR, SSCP, RFLP analysis, AFLP analysis, fluorescent labeling, and the like, may be used to identify engrafted cell-specific nucleotide sequences in a tissue sample from the recipient. Such nucleic acid detection and analysis methods are well-known in the art. In one embodiment, engraftment may be determined by the appearance of engrafted cell-specific nucleic acids in a tissue sample from a recipient, which are distinguishable from background. The tissue sample analyzed may be, for example, a biopsy (e.g., bone marrow aspirate) or a blood sample.

In one embodiment, a sample of peripheral blood is taken from an individual immediately prior to a medical procedure, e.g., myeloablation. After the procedure, the composition comprising AMDACs and platelet rich plasma is administered to the individual. At least once post-administration, a second sample of peripheral blood is taken. An STR profile is obtained for both samples, e.g., using PCR primers for markers (alleles) available from, e.g., LabCorp (Laboratory Corporation of America). A difference in the number or characteristics of the markers (alleles) post-administration indicates that engraftment has taken place.

Engraftment can also be demonstrated by detection of re-emergence of neutrophils.

In another example, engrafted cell-specific markers may be detected in a tissue sample from the recipient using antibodies directed to markers specific to either the transplanted AMDACs, or cells into which the AMDACs would be expected to differentiate. In certain embodiments, engraftment of a combination of AMDACs and platelet rich plasma may be assessed by FACS analysis to determine the presence of any cellular marker described herein as being displayed by AMDACs, e.g., by adding the appropriate antibody and allowing binding; washing (e.g., with PBS); fixing the cells (e.g., with 1% paraformaldehyde); and analyzing on an appropriate FACS apparatus (e.g., a FACSCalibur flow cytometer (Becton Dickinson)). Where AMDACs and/or platelet rich plasma are from an individual of a different sex than a recipient, e.g., male donor and female recipient, engraftment can be determined by detection of sex-specific markers, e.g., Y-chromosome-specific markers. AMDACs may also be genetically modified to express a unique marker or nucleic acid sequence that facilitates identification, e.g., an RFLP marker, expression of β-galactosidase or green fluorescent protein, or the like.

The degree of engraftment may be assessed by any means known in the art. In one embodiment, the degree of engraftment is assessed by a grading system as follows, which uses a thin section of fixed and antibody-bound tissue from the transplant recipient. In this example grading system, engraftment is graded as follows: 0=no positive cells (that is, no cells bound by an antibody specific to an engrafted cell); 0.5=one or two positive cells, perhaps positive, but difficult to differentiate from background or non-specific staining; 1=2-20 scattered positive cells; 2=approximately 20-100 scattered or clustered positive cells throughout the tissue; 3=more than 100 positive cells comprising less than 50% of the tissue; 4=more than 50% of cells are positive. In specific embodiments, engraftment is determined where greater than 0.5%, 1%, 2%, 3%, 4%, 5%, 7.5%, 10%, 15%, 20% or greater of the cells are positively stained.

### 5.4 Methods of Treatment Using Amnion Derived Adherent Cells and Platelet-Rich Plasma

Provided herein are methods of treating an individual having a disease or disorder comprising administering to the individual a composition comprising amnion derived adherent cells and platelet-rich plasma. The compositions comprising AMDACs and platelet rich plasma provided herein can be used to treat individuals exhibiting a variety of disease states or conditions that would benefit from reduced inflammation, promotion of angiogenesis, modulation of an immune response, and enhanced healing. Examples of such disease states or conditions include, but are not limited to: repetitive use injuries, such as lateral epicondylitis (tennis elbow) and carpal tunnel syndrome; sports injuries, such as torn ligaments and tendons, torn rotator cuffs and meniscal tears; degenerative joint conditions such as osteoarthritis of the hip, knee, shoulder, elbow; disease of or trauma to a joint; disease states or conditions characterized by a disruption of blood flow in the peripheral vasculature, such as peripheral arterial disease (PAD), e.g., critical limb ischemia (CLI); neuropathic pain; dermatological conditions, e.g., for the treatment of wounds (external and internal), acute and chronic wounds, e.g., various ulcers, congenital wounds, burns, and skin conditions, e.g., skin lesions; and bone related uses and the treatment of orthopedic defects, e.g., disc herniation and degenerative disc disease. Thus, in another aspect, provided herein is a method of treating an individual suffering from a disease or condition that would benefit from reduced inflammation, immune modulation, promotion of angiogenesis, and enhanced healing, comprising administering a therapeutically effective amount of a composition comprising AMDACs and platelet rich plasma, as described herein, to said individual in an amount and for a time sufficient for detectable improvement of said disease or condition.

In certain embodiments, the individual is an animal, preferably a mammal, more preferably a non-human primate. In certain embodiments, the individual is a human patient. The individual can be a male or female subject. In certain embodiments, the subject is a non-human animal, such as, for instance, a cow, sheep, goat, horse, dog, cat, rabbit, rat or mouse.

In one embodiment, the individual is administered a dose of a composition comprising platelet-rich plasma and about 300 million AMDACs. Dosage, however, can vary according to the individual's physical characteristics, e.g., weight, and can range from 1 million to 10 billion AMDACs per dose, preferably between 10 million and 1 billion per dose, or between 100 million and 500 million AMDACs per dose. In other embodiments, transplantation of said composition comprising AMDACs combined with platelet rich plasma prolongs localization of the AMDACs at the site of injection or implantation at least, or at, 1, 2, 3, 4, 5, 6, 7, 8, 9, 10, 11, 12, 13, 14, 15, 16, 17, 18, 19, 20, 21, 22, 23, or 24 hours, or 1, 2, 3, 4, 5, 6, 7, 8, 9, 10, 11, 12, 13, 14, 15, 16, 17, 18, 19, 20, or 21 days post-transplant, relative to transplantation of AMDACs not combined with platelet rich plasma. In another more specific embodiment, said composition comprising AMDACs combined with platelet rich plasma prolongs localization of the AMDACs at the site of injection or implantation at least, or at most, more than 21 days post-transplant. In specific embodiments, said composition comprising AMDACs combined with platelet rich plasma prolongs localization of the AMDACs at the site of injection or implantation at least, or at most, more than 25, 30, 35, 40, 45, 50, 55 weeks, or 1 year or longer post-transplant.

5.4.1 Treatment of Vascular or Cardiac Conditions

In one aspect, provided herein are methods for treating an individual having a vascular disease or cardiac medical condition comprising administering to said individual a therapeutically-effective amount of a composition comprising AMDACs and platelet rich plasma. In a specific embodiment, the method comprises evaluating the individual for one or more indicia of improvement in vascular or cardiac function.

In one embodiment, the medical condition is a cardiomyopathy. In specific embodiments, the cardiomyopathy is either idiopathic or a cardiomyopathy with a known cause. In other specific embodiments, the cardiomyopathy is either ischemic or nonischemic in nature. In another embodiments, the vascular disease or cardiac medical condition comprises one or more of angioplasty, aneurysm, angina (angina pectoris), aortic stenosis, aortitis, arrhythmias, arteriosclerosis, arteritis, asymmetric septal hypertrophy (ASH), atherosclerosis, atrial fibrillation and flutter, bacterial endocarditis, Barlow's Syndrome (mitral valve prolapse), bradycardia, Buerger's Disease (thromboangiitis obliterans), cardiomegaly, cardiomyopathy, carditis, carotid artery disease, coarctation of the aorta, congenital heart diseases (congenital heart defects), congestive heart failure (heart failure), coronary artery disease, Eisenmenger's Syndrome, embolism, endocarditis, erythromelalgia, fibrillation, fibromuscular dysplasia, heart block, heart murmur, hypertension, hypotension, idiopathic infantile arterial calcification, Kawasaki Disease (mucocutaneous lymph node syndrome, mucocutaneous lymph node disease, infantile polyarteritis), metabolic syndrome, microvascular angina, myocardial infarction (heart attacks), myocarditis, paroxysmal atrial tachycardia (PAT), periarteritis nodosa (polyarteritis, polyarteritis nodosa), pericarditis, diabetic vasculopathy, phlebitis, pulmonary valve stenosis (pulmonic stenosis), Raynaud's Disease, renal artery stenosis, renovascular hypertension, rheumatic heart disease, septal defects, silent ischemia, syndrome X, tachycardia, Takayasu's Arteritis, Tetralogy of Fallot, transposition of the great vessels, tricuspid atresia, truncus arteriosus, valvular heart disease, varicose ulcers, varicose veins, vasculitis, ventricular septal defect, Wolff-Parkinson-White Syndrome, or endocardial cushion defect.

In another specific embodiment, the vascular disease is peripheral vascular disease, e.g., critical limb ischemia (acute limb ischemia).

In other embodiments, the vascular disease or cardiac medical condition comprises one or more of acute rheumatic fever, acute rheumatic pericarditis, acute rheumatic endocarditis, acute rheumatic myocarditis, chronic rheumatic heart diseases, diseases of the mitral valve, mitral stenosis, rheumatic mitral insufficiency, diseases of aortic valve, diseases of other endocardial structures, ischemic heart disease (acute and subacute), angina pectoris, diseases of pulmonary circulation (acute pulmonary heart disease, pulmonary embolism, chronic pulmonary heart disease), kyphoscoliotic heart disease, myocarditis, endocarditis, endomyocardial fibrosis, endocardial fibroelastosis, atrioventricular block, cardiac dysrhythmias, myocardial degeneration, diseases of the vascular system including cerebrovascular disease, occlusion and stenosis of precerebral arteries, occlusion of cerebral arteries, diseases of arteries, arterioles and capillaries (atherosclerosis, aneurysm), or diseases of veins and lymphatic vessels.

In one embodiment, treatment comprises treatment of a patient with a cardiomyopathy with a composition comprising AMDACs and platelet rich plasma, either with or without another cell type. In other preferred embodiments, the individual experiences benefits from the therapy, for example from the ability of the cells to support the growth of other cells, including stem cells or progenitor cells present in the heart, from the tissue ingrowth or vascularization of the tissue, and from the presence of beneficial cellular factors, chemokines, cytokines and the like, but the cells do not integrate or multiply in the patient. In another embodiment, the individual benefits from the therapeutic treatment with the cells, but the cells do not survive for a prolonged period in the patient. In one embodiment, the cells gradually decline in number, viability or biochemical activity. In other embodiments, the decline in cells may be preceded by a period of activity, for example growth, division, or biochemical activity. In other embodiments, senescent, nonviable or even dead cells are able to have a beneficial therapeutic effect.

In another embodiment, improvement in said individual having a vascular disease or cardiac medical condition, wherein the individual has been administered the composition comprising AMDACs and platelet rich plasma, can be assessed or demonstrated by detectable improvement in one or more, indicia of cardiac function, for example, demonstration of detectable improvement in one or more of chest cardiac output (CO), cardiac index (CI), pulmonary artery wedge pressures (PAWP), and cardiac index (CI), % fractional shortening (% FS), ejection fraction (EF), left ventricular ejection fraction (LVEF); left ventricular end diastolic diameter (LVEDD), left ventricular end systolic diameter (LVESD), contractility (e.g. dP/dt), pressure-volume loops, measurements of cardiac work, an increase in atrial or ventricular functioning; an increase in pumping efficiency, a decrease in the rate of loss of pumping efficiency, a decrease in loss of hemodynamic functioning; and a decrease in complications associated with cardiomyopathy, as compared to the individual prior to administration of amnion derived adherent cells.

Improvement in an individual receiving the composition comprising AMDACs and platelet rich plasma can also be assessed by subjective metrics, e.g., the individual's self-assessment about his or her state of health following administration.

Success of administration of the composition is not, in certain embodiments, based on survival in the individual of the administered amnion derived adherent cells. Success is, instead, based on one or more metrics of improvement in cardiac or circulatory health, as noted above. Thus, the cells need not integrate into the patient's heart, or into blood vessels.

In certain embodiments, the methods of treatment provided herein comprise inducing the amnion derived adherent cells in said composition, either before or after combining with platelet rich plasma, to differentiate along mesenchymal lineage, e.g., towards a cardiomyogenic, angiogenic or vasculogenic phenotype, or into cells such as myocytes, cardiomyocytes, endothelial cells, myocardial cells, epicardial cells, vascular endothelial cells, smooth muscle cells (e.g. vascular smooth muscle cells).

Administration of a composition comprising AMDACs and platelet rich plasma, to an individual in need thereof, can be accomplished, e.g., by transplantation, implantation (e.g. of the cells themselves or the cells as part of a matrix-cell combination), injection (e.g., directly to the site of the disease or condition, for example, directly to an ischemic site in the heart of an individual who has had a myocardial infarction), infusion, delivery via catheter, or any other means known in the art for providing cell therapy.

In one embodiment, the composition comprising AMDACs and platelet rich plasma are provided to an individual in need thereof, for example, by injection into one or more sites in the individual. In a specific embodiment, the therapeutic cell compositions are provided by intracardiac injection, e.g., to an ischemic area in the heart. In other specific embodiments, the cells are injected onto the surface of the heart, into an adjacent area, or even to a more remote area. In preferred embodiments, the cells can home to the diseased or injured area.

An individual having a disease or condition of the coronary or vascular systems can be administered a composition comprising AMDACs and platelet rich plasma at any time the cells would be therapeutically beneficial. In certain embodiments, for example, the composition comprising AMDACs and platelet rich plasma are administered within 1, 2, 3, 4, 5, 6, 7, 8, 9, 10, 11, 12, 13, 14, 15, 16, 17, 18, 19, 20, 21, 22, 23, or 24 hours, or 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days of the myocardial infarction. Administration proximal in time to a myocardial infarction, e.g., within 1-3 or 1-7 days, is preferable to administration distal in time, e.g., after 3 or 7 days after a myocardial infarction. In other embodiments, the composition is administered within 1, 2, 3, 4, 5, 6, 7, 8, 9, 10, 11, 12, 13, 14, 15, 16, 17, 18, 19, 20, 21, 22, 23, or 24 hours, or 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days of initial diagnosis of the disease or condition.

Also provided herein are kits for use in the treatment of myocardial infarction. The kits provide the composition comprising AMDACs and platelet rich plasma which can be prepared in a pharmaceutically acceptable form, for example by mixing with a pharmaceutically acceptable carrier, and an applicator, along with instructions for use. Ideally the kit can be used in the field, for example in a physician's office, or by an emergency care provider to be applied to a patient diagnosed as having had a myocardial infarction or similar cardiac event.

In specific embodiments of the methods of treatment provided herein, the composition comprising AMDACs and platelet rich plasma is administered with stem cells (that is, stem cells that are not amnion derived adherent cells), myoblasts, myocytes, cardiomyoblasts, cardiomyocytes, or progenitors of myoblasts, myocytes, cardiomyoblasts, and/or cardiomyocytes.

In a specific embodiment, the methods of treatment provided herein comprise administering a composition comprising AMDACs and platelet rich plasma to a patient with a disease of the heart or circulatory system; and evaluating the patient for improvements in cardiac function, wherein the therapeutic cell composition is administered as a matrix-cell complex. In certain embodiments, the matrix is a scaffold, preferably bioabsorbable, comprising at least the cells.

Amnion derived adherent cells may be differentiated along cardiogenic, angiogenic, hemangiogenic, or vasculogenic pathways or lineages by culture of the cells in the presence of factors comprising at least one of a demethylation agent, a BMP, FGF, Wnt factor protein, Hedgehog, and/or anti-Wnt factors.

Inclusion of demethylation agents tends to allow the cells to differentiate along mesenchymal lines, toward a cardiomyogenic pathway. Differentiation can be determined by, for example, expression of at least one of cardiomyosin, skeletal myosin, or GATA4; or by the acquisition of a beating rhythm, spontaneous or otherwise induced; or by the ability to integrate at least partially into a patient's cardiac muscle without inducing arrhythmias. Demethylation agents that can be used to initiate such differentiation include, but are not limited to, 5-azacytidine, 5-aza-2′-deoxycytidine, dimethylsulfoxide, chelerythrine chloride, retinoic acid or salts thereof, 2-amino-4-(ethylthio)butyric acid, procainamide, and procaine.

In certain embodiments herein, cells induced with one or more factors as identified above may become cardiomyogenic, angiogenic, hemangiogenic, or vasculogenic cells, or progenitors. Preferably at least some of the cells can integrate at least partially into a recipient's cardiovascular system, including but not limited to heart muscle, vascular and other structures of the heart, cardiac or peripheral blood vessels, and the like. In certain other embodiments, the differentiated amnion derived adherent cells differentiate into cells acquiring two or more of the indicia of cardiomyogenic cells or their progenitors, and able to partially or fully integrate into a recipient's heart or vasculature. In specific embodiments, the cells, which administered to an individual, result in no increase in arrhythmias, heart defects, blood vessel defects or other anomalies of the individual's circulatory system or health. In certain embodiments, the amnion derived adherent cells act to promote the differentiation of stem cells naturally present in the patient's cardiac muscle, blood vessels, blood and the like to themselves differentiate into for example, cardiomyocytes, or at least along cardiomyogenic, angiogenic, hemangiogenic, or vasculogenic lines.

The composition comprising AMDACs and platelet rich plasma can be provided therapeutically or prophylactically to an individual, e.g., an individual having a disease, disorder or condition of or affecting, the heart or circulatory system. Such diseases, disorders or conditions can include congestive heart failure due to atherosclerosis, cardiomyopathy, or cardiac injury, e.g., an ischemic injury, such as from myocardial infarction or wound (acute or chronic).

The composition comprising AMDACs and platelet rich plasma may comprise another therapeutic agent, such as insulin-like growth factor (IGF), platelet-derived growth factor (PDGF), epidermal growth factor (EGF), fibroblast growth factor (FGF), vascular endothelial growth factor (VEGF), hepatocyte growth factor (HGF), IL-8, an antithrombogenic agent (e.g., heparin, heparin derivatives, urokinase, and PPack (dextrophenylalanine proline arginine chloromethylketone); antithrombin compounds, platelet receptor antagonists, anti-thrombin antibodies, anti-platelet receptor antibodies, aspirin, dipyridamole, protamine, hirudin, prostaglandin inhibitors, and/or platelet inhibitors), an antiapoptotic agent (e.g., EPO, EPO derivatives and analogs, and their salts, TPO, IGF-I, IGF-II, hepatocyte growth factor (HGF), or caspase inhibitors), an anti-inflammatory agent (e.g., P38 MAP kinase inhibitors, statins, IL-6 and IL-1 inhibitors, Pemirolast, Tranilast, Remicade, Sirolimus, nonsteroidal anti-inflammatory compounds, for example, acetylsalicylic acid, ibuprofen, Tepoxalin, Tolmetin, or Suprofen), an immunosuppressive or immunomodulatory agent (e.g., calcineurin inhibitors, for example cyclosporine, Tacrolimus, mTOR inhibitors such as Sirolimus or Everolimus; anti-proliferatives such as azathioprine and mycophenolate mofetil; corticosteroids, e.g., prednisolone or hydrocortisone; antibodies such as monoclonal anti-IL-2Rα receptor antibodies, Basiliximab, Daclizuma, polyclonal anti-T-cell antibodies such as anti-thymocyte globulin (ATG), anti-lymphocyte globulin (ALG), and the monoclonal anti-T cell antibody OKT3, or adherent placental stem cells as described in U.S. Pat. No. 7,468,276, and U.S. Patent Application Publication No. and 2007/0275362, the disclosures of which are incorporated herein by reference in their entireties), and/or an antioxidant (e.g., probucol; vitamins A, C, and E, coenzyme Q-10, glutathione, L cysteine, N-acetylcysteine, or antioxidant derivative, analogs or salts of the foregoing). In certain embodiments, composition comprising AMDACs and platelet rich plasma further comprises one or more additional cell types, e.g., adult cells (for example, fibroblasts or endodermal cells), or stem or progenitor cells. Such therapeutic agents and/or one or more additional cells, can be administered to an individual in need thereof individually or in combinations or two or more such compounds or agents.

In a specific embodiment, the disease state or condition treatable with a therapeutically effective amount of a composition comprising amnion derived adherent cells and platelet-rich plasma is critical limb ischemia (CLI). Thus, in another aspect, provided herein is a method of treating an individual having CLI, comprising administering to the individual a therapeutically-effective amount of a composition comprising AMDACs, as described herein, and platelet rich plasma.

In certain embodiments, said CLI is a severe blockage in the arteries of the lower extremities, which markedly reduces blood-flow. In another more specific embodiment, said CLI is characterized by ischemic rest pain, severe pain in the legs and feet while a person is not moving, non-healing sores on the feet or legs, pain or numbness in the feet, shiny, smooth, dry skin of the legs or feet, thickening of the toenails, absent or diminished pulse in the legs or feet, open sores, skin infections or ulcers that will not heal, and/or dry gangrene (dry, black skin) of the legs or feet. In another specific embodiment, CLI can lead to loss of digits and or whole limbs. In another specific embodiment of the method, administration of said therapeutically effective amount of a composition comprising amnion derived adherent cells and platelet-rich plasma results in elimination of, a detectable improvement in, lessening of the severity of, or slowing of the progression of one or more symptoms of, loss of limb function and or oxygen deprivation (hypoxia/anoxia) attributable to, a disruption of the flow of blood in or around the limb of said individual. In another specific embodiment, said therapeutically effective amount of a composition comprising amnion derived adherent cells and platelet-rich plasma is administered to said individual prophylactically, e.g., to reduce or eliminate tissue damage caused by a second or subsequent disruption of flow of blood in or around the limb following said disruption of flow of blood.

In some embodiments, the CLI results from an acute condition such as an embolus or thrombosis. In some embodiments, the CLI is the end result of arterial occlusive disease, e.g., atherosclerosis. In particular embodiments, the CLI results from atherosclerosis in association with hypertension, hypercholesterolemia, cigarette smoking and diabetes. In some embodiments, the CLI results from Buerger's disease, thromboangiitis obliterans, or arteritis.

In some embodiments, the CLI is characterized by claudication, wherein narrowed vessels cannot supply sufficient blood flow to exercising leg muscles, which is brought on by exercise and relieved by rest. In some embodiments, the CLI is characterized by burning pain in the ball of the foot and toes that is worse at night when the individual is in bed. In some embodiments, the CLI is characterized by progressive gangrene, rapidly enlarging wounds and/or continuous ischemic rest pain. In some embodiments, the CLI is characterized by an ankle-brachial index of 0.4 or less, more than two weeks of recurrent foot pain at rest that requires regular use of analgesics and is associated with an ankle systolic pressure of 50 mm Hg or less, or a toe systolic pressure of 30 mm Hg or less, and/or a nonhealing wound or gangrene of the foot or toes, with similar hemodynamic measurements. Generally, a wound is considered to be nonhealing if it fails to respond to a four- to 12-week trial of conservative therapy such as regular dressing changes, avoidance of trauma, treatment of infection and debridement of necrotic tissue.

The methods for treating CLI provided herein further encompass treating CLI by administering a therapeutically effective amount of a composition comprising amnion derived adherent cells and platelet rich plasma, in conjunction with one or more therapies or treatments used in the course of treating CLI. The one or more additional therapies may be used prior to, concurrent with, or after administration of the composition comprising amnion derived adherent cells and platelet rich plasma. In some embodiments, the one or more additional therapies comprise operative intervention. In some embodiments, the operative intervention comprises surgical revascularization.

In some embodiments, the surgical revascularization comprises minimally invasive endovascular therapy. In some embodiments, the endovascular therapy comprises puncture of the groin, under local anesthesia, with insertion of a catheter into the artery in the groin which will allow access to the diseased portion of the artery, e.g., a site of plaque localization. In some embodiments, the endovascular therapy comprises angioplasty, i.e., insertion of a small balloon through a puncture in the groin, wherein the balloon is inflated one or more times, using a saline solution, to open the artery. In some embodiments, the endovascular therapy comprises insertion of a cutting balloon, i.e., a balloon imbedded with micro-blades is used to dilate the diseased area, wherein the blades cut the surface of the plaque, reducing the force necessary to dilate the vessel. In some embodiments, the endovascular therapy comprises insertion of a cold balloon, i.e., cryoplasty, wherein instead of using saline, the balloon is inflated using nitrous oxide which freezes the plaque. In some embodiments, the endovascular therapy comprises insertion of one or stents, i.e., metal mesh tubes that provide scaffolding, for example, after an artery has been opened using a balloon angioplasty. In some embodiments, the stent is a balloon-expanded stent. In some embodiments, the stent is a self-expanding stent. In some embodiments, the endovascular therapy comprises laser atherectomy, wherein small bits of plaque are vaporized by the tip of a laser probe. In some embodiments, the endovascular therapy comprises directional atherectomy, wherein a catheter with a rotating cutting blade is used to physically remove plaque from the artery, opening the flow channel.

5.4.2 Wound Healing Applications

In another specific embodiment of the methods of treatment described herein, a composition comprising amnion derived adherent cells and platelet-rich plasma is used for the treatment of a wound, including but not limited to: an epidermal wound, skin wound, chronic wound, acute wound, external wound, internal wound, and a congenital wound (e.g., dystrophic epidermolysis bullosa).

In other embodiments, a composition comprising amnion derived adherent cells and platelet-rich plasma is administered to an individual for the treatment of a wound infection, e.g., a wound infection followed by a breakdown of a surgical or traumatic wound. The compositions comprising amnion derived adherent cells and platelet-rich plasma described herein have therapeutic utility in the treatment of wound infections from any microorganism known in the art, e.g., microorganisms that infect wounds originating from within the human body, which is a known reservoir for pathogenic organisms, or from environmental origin. A non-limiting example of the microorganisms, the growth of which in wounds may be reduced or prevented by the methods and compositions described herein are Staphylococcus aureus, S. epidermidis, beta haemolytic streptococci, Escherichia coli, Klebsiella and Pseudomonas species, and among the anaerobic bacteria, the Clostridium welchii or C. tartium, which are the cause of gas gangrene, mainly in deep traumatic wounds.

In other embodiments, a composition comprising amnion derived adherent cells and platelet-rich plasma is administered for the treatment of burns, including but not limited to, first-degree burns, second-degree burns (partial thickness burns), third degree burns (full thickness burns), infection of burn wounds, infection of excised and unexcised burn wounds, infection of grafted wound, infection of donor site, loss of epithelium from a previously grafted or healed burn wound or skin graft donor site, and burn wound impetigo.

In particular, the compositions comprising amnion derived adherent cells and platelet-rich plasma described herein have enhanced utility in the treatment of ulcers, e.g., leg ulcers. In various embodiments, said leg ulcer can be, for example, a venous leg ulcer, arterial leg ulcer, diabetic leg ulcer, decubitus ulcer, or split thickness skin grafted ulcer or wound. In this context, “treatment of a leg ulcer” comprises contacting the leg ulcer with an amount of a composition comprising amnion derived adherent cells and platelet-rich plasma effective to improve at least one aspect of the leg ulcer. As used herein, “aspect of the leg ulcer” includes objectively measurable parameters such as ulcer size, depth or area, degree of inflammation, ingrowth of epithelial and/or mesodermal tissue, gene expression within the ulcerated tissue that is correlated with the healing process, quality and extent of scarring etc., and subjectively measurable parameters, such as patient well-being, perception of improvement, perception of lessening of pain or discomfort associated with the ulcer, patient perception that treatment is successful, and the like.

5.4.2.1 Venous Leg Ulcers

Provided herein are methods for the treatment of venous leg ulcers comprising administering an amount of a composition comprising amnion derived adherent cells and platelet-rich plasma effective to improve at least one aspect of the venous leg ulcer. Venous leg ulcers, also known as venous stasis ulcers or venous insufficiency ulcers, a type of chronic or non-healing wound, are widely prevalent in the United States, with approximately 7 million people, usually the elderly, afflicted. Worldwide, it is estimated that 1-1.3% of individuals suffer from venous leg ulcers. Approximately 70% of all leg ulcers are venous ulcers. Venous leg ulcers are often located in the distal third of the leg known as the gaiter region, and typically on the inside of the leg. The ulcer is usually painless unless infected. Venous leg ulcers typically occur because the valves connecting the superficial and deep veins fail to function properly. Failure of these valves causes blood to flow from the deep veins back out to the superficial veins. This inappropriate flow, together with the effects of gravity, causes swelling and progression to damage of lower leg tissues. Patients with venous leg ulcers often have a history of deep vein thrombosis, leg injury, obesity, phlebitis, prior vein surgery, and lifestyles that require prolonged standing. Other factors may contribute to the chronicity of venous leg ulcers, including poor circulation, often caused by arteriosclerosis; disorders of clotting and circulation that may or may not be related to atherosclerosis; diabetes; renal (kidney) failure; hypertension (treated or untreated); lymphedema (buildup of fluid that causes swelling in the legs or feet); inflammatory diseases such as vasculitis, lupus, scleroderma or other rheumatological conditions; medical conditions such as high cholesterol, heart disease, high blood pressure, sickle cell anemia, or bowel disorders; a history of smoking (either current or past); pressure caused by lying in one position for too long; genetics (predisposition for venous disease); malignancy (tumor or cancerous mass); infections; and certain medications.

Thus, in another embodiment, provided herein is a method of treating a venous leg ulcer comprising contacting the venous leg ulcer with an amount of a composition comprising amnion derived adherent cells and platelet-rich plasma sufficient to improve at least one aspect of the venous leg ulcer. In another specific embodiment, the method additionally comprises treating an underlying cause of the venous leg ulcer.

The methods for treating a venous leg ulcer provided herein further encompass treating the venous leg ulcer by administering a therapeutically effective amount of a composition comprising amnion derived adherent cells and platelet rich plasma, in conjunction with one or more therapies or treatments used in the course of treating a venous leg ulcer. The one or more additional therapies may be used prior to, concurrent with, or after administration of the composition comprising amnion derived adherent cells and platelet rich plasma. In some embodiments, the one or more additional therapies comprise compression of the leg to minimize edema or swelling. In some embodiments, compression treatments include wearing therapeutic compression stockings, multilayer compression wraps, or wrapping an ACE bandage or dressing from the toes or foot to the area below the knee.

5.4.2.2 Other Leg Ulcer Types

Arterial leg ulcers are caused by an insufficiency in one or more arteries' ability to deliver blood to the lower leg, most often due to atherosclerosis. Arterial ulcers are usually found on the feet, particularly the heels or toes, and the borders of the ulcer appear as though they have been ‘punched out’. Arterial ulcers are frequently painful. This pain is relieved when the legs are lowered with feet on the floor as gravity causes more blood to flow into the legs. Arterial ulcers are usually associated with cold white or bluish, shiny feet.

The treatment of arterial leg ulcers contrasts to the treatment of venous leg ulcers in that compression is contraindicated, as compression tends to exacerbate an already-poor blood supply, and debridement is limited, if indicated at all. Thus, in another embodiment, provided herein is a method of treating an arterial leg ulcer comprising treating the underlying cause of the arterial leg ulcer, e.g., arteriosclerosis, and contacting the arterial leg ulcer with an amount of a composition comprising amnion derived adherent cells and platelet-rich plasma sufficient to improve at least one aspect of the arterial leg ulcer. In a specific embodiment, the method of treating does not comprise compression therapy.

Diabetic foot ulcers are ulcers that occur as a result of complications from diabetes. Diabetic ulcers are typically caused by the combination of small arterial blockage and nerve damage, and are most common on the foot, though they may occur in other areas affected by neuropathy and pressure. Diabetic ulcers have characteristics similar to arterial ulcers but tend to be located over pressure points such as heels, balls of the feet, tips of toes, between toes or anywhere bony prominences rub against bed sheets, socks or shoes.

Treatment of diabetic leg ulcers is generally similar to the treatment of venous leg ulcers, though generally without compression; additionally, the underlying diabetes is treated or managed. Thus, in another embodiment, provided herein is a method of treating a diabetic leg ulcer comprising treating the underlying diabetes, and contacting the diabetic leg ulcer with an amount of a composition comprising amnion derived adherent cells and platelet-rich plasma sufficient to improve at least one aspect of the diabetic leg ulcer.

Decubitus ulcers, commonly called bedsores or pressure ulcers, can range from a very mild pink coloration of the skin, which disappears in a few hours after pressure is relieved on the area to a very deep wound extending into the bone. Decubitus ulcers occur frequently with patients subject to prolonged bedrest, e.g., quadriplegics and paraplegics who suffer skin loss due to the effects of localized pressure. The resulting pressure sores exhibit dermal erosion and loss of the epidermis and skin appendages. Factors known to be associated with the development of decubitus ulcers include advanced age, immobility, poor nutrition, and incontinence. Stage 1 decubitus ulcers exhibit nonblanchable erythema of intact skin. Stage 2 decubitus ulcers exhibit superficial or partial thickness skin loss. Stage 3 decubitus ulcers exhibit full thickness skin loss with subcutaneous damage. The ulcer extends down to underlying fascia, and presents as a deep crater. Finally, stage 4 decubitus ulcers exhibit full thickness skin loss with extensive destruction, tissue necrosis, and damage to the underlying muscle, bone, tendon or joint capsule. Thus, in another embodiment, provided herein is a method of treating a decubitus leg ulcer comprising treating the underlying diabetes, and contacting the decubitus leg ulcer with an amount of a composition comprising amnion derived adherent cells and platelet-rich plasma sufficient to improve at least one aspect of the decubitus leg ulcer.

The methods of treatment provided herein further encompass treating a leg ulcer by administering a composition comprising amnion derived adherent cells and platelet rich plasma in conjunction with one or more therapies or treatments used in the course of treating a leg ulcer. The one or more additional therapies may be used prior to, concurrent with, or after administration of the composition comprising amnion derived adherent cells and platelet rich plasma. A composition comprising amnion derived adherent cells and platelet rich plasma, and one or more additional therapies, may be used where the composition comprising amnion derived adherent cells and platelet rich plasma, alone, or the one or more additional therapies, alone, would be insufficient to measurably improve, maintain, or lessen the worsening of, one or more aspects of a leg ulcer. In specific embodiments, the one or more additional therapies comprise, without limitation, treatment of the leg ulcer with a wound healing agent (e.g., PDGF, REGRANEX®); administration of an anti-inflammatory compound; administration of a pain medication; administration of an antibiotic; administration of an anti-platelet or anti-clotting medication; application of a prosthetic; application of a dressing (e.g., moist to moist dressings; hydrogels/hydrocolloids; alginate dressings; collagen-based wound dressings; antimicrobial dressings; composite dressings; synthetic skin substitutes, etc.), and the like. In another embodiment, the additional therapy comprises contacting the leg ulcer with honey. For any of the above embodiments, in a specific embodiment, the leg ulcer is a venous leg ulcer, a decubitus ulcer, a diabetic ulcer, or an arterial leg ulcer.

In another specific embodiment, the additional therapy is a pain medication. In another embodiment, therefore, the method of treating a leg ulcer comprises contacting the leg ulcer with a composition comprising amnion derived adherent cells and platelet rich plasma, and administering a pain medication to lessen or eliminate leg ulcer pain. In a specific embodiment, the pain medication is a topical pain medication.

In another specific embodiment, the additional therapy is an anti-infective agent. Preferably, the anti-infective agent is one that is not cytotoxic to healthy tissues surrounding and underlying the leg ulcer; thus, compounds such as iodine and bleach are disfavored. Thus, treatment of the leg ulcer, in one embodiment, comprises contacting the leg ulcer with a composition comprising amnion derived adherent cells and platelet rich plasma, and administering an anti-infective agent. The anti-infective agent may be administered by any route, e.g., topically, orally, buccally, intravenously, intramuscularly, anally, etc. In a specific example, the anti-infective agent is an antibiotic, a bacteriostatic agent, antiviral compound, a virustatic agent, antifungal compound, a fungistatic agent, or an antimicrobial compound. In another specific embodiment, the anti-infective agent is ionic silver. In a more specific embodiment, the ionic silver is contained within a hydrogel. In specific embodiments, the leg ulcer is a venous leg ulcer, arterial leg ulcer, decubitus ulcer, or diabetic ulcer.

5.4.3 Treatment of Chronic Pain

Chronic pain, e.g., neuropathic pain, a condition that afflicts at least 30% of Americans, is caused, e.g., by disorders of the nervous system, also known as neuropathy, and can be accompanied by, or caused by, tissue damage, including nerve fibers that are damaged, dysfunction or injured. Neuropathic pain may also be caused by, e.g. pathologic lesions, neurodegeneration processes, or prolonged dysfunction of parts of the peripheral or central nervous system. However, neuropathic pain can also be present when no discernible tissue damage is evident.

Neuropathic pain is generally regarded as having two components: central plasticity, e.g., as a result of changes in receptor population or receptor sensitivity at any level of the CNS, and changes in peripheral nerves, neurons and microglial, which are mediators of central sensitization of the spinal cord. Such sensitization is known to play a major role in mediating chronic inflammatory pain and neuropathic pain.

Thus, in another aspect, provided herein is a method of treating an individual having chronic pain comprising administering to the individual a therapeutically-effective amount of a composition comprising amnion derived adherent cells, as described herein, and platelet-rich plasma. In a specific embodiment, the chronic pain, e.g., neuropathic pain, is, or is caused by, neuritis (e.g., polyneuritis, brachial neuritis, optic neuritis, vestibular neuritis, cranial neuritis, or arsenic neuritis), diabetes mellitus (e.g., diabetic neuropathy), peripheral neuropathy, reflex sympathetic dystrophy syndrome, phantom limb pain, post-amputation pain, postherpetic neuralgia, shingles, central pain syndrome (pain caused, e.g., by damage to the brain, spinal cord and/or brainstem), Guillain-Barre Syndrome, degenerative disc disease, cancer, multiple sclerosis, kidney disorders, alcoholism, human immunodeficiency virus-related neuropathy, Wartenberg's Migratory Sensory Neuropathy, fibromyalgia syndrome, causalgia, spinal cord injury, or exposure to a chemical agent, e.g., trichloroethylene or dapsone (diaminyl-diphenyl sulfone). In specific embodiments, the peripheral neuropathy is mononeuropathy (damage to a single peripheral nerve); polyneuropathy (damage to more than one peripheral nerve, frequently sited in different parts of the body), mononeuritis multiplex (simultaneous or sequential damage to noncontiguous nerve trunks), or autonomic neuropathy. Peripheral neuropathy, e.g., mononeuritis multiplex, may be caused by, e.g., diabetes mellitus, vasculitis (e.g., polyarteritis nodosa, Wegener granulomatosis, or Churg-Strauss syndrome), rheumatoid arthritis, lupus erythematosus (SLE), sarcoidosis, an amyloidosis, or cryoglobulinemia.

As used herein, “therapeutically effective amount” is an amount of the composition sufficient to result in a detectable, or reportable, lessening of said chronic pain. The lessening of pain may be, e.g., self-reported by the individual, or may be determined by physiological signs responsive to pain, e.g. elevated blood pressure, anxiety, and the like. Levels of neuropathic pain may be assessed, e.g., by the Visual Analog Scale (VAS). Numeric Pain Intensity Scale, Graphic Rating Scale, Verbal Rating Scale, Pain Faces Scale (Faces Pain Scale), Numeric Pain Intensity & Pain Distress Scales, Brief Pain Inventory (BPI), Memorial Pain Assessment, Alder Hey Triage Pain Score, Dallas Pain Questionnaire, Dolorimeter Pain Index (DPI), Face Legs Activity Cry Consolability Scale, Lequesne Scale, McGill Pain Questionnaire (MPQ), Descriptor differential scale (DDS), Neck Pain and disability Scale (NPAD), Numerical 11-Point Box (BS-11), Roland-Morris Back Pain Questionnaire, or the Wong-Baker FACES Pain Rating Scale. An improvement after administration of the composition to the individual in one or more of these assessments of pain is considered therapeutically effective.

In a specific embodiment, the composition comprising amnion derived adherent cells and PRP is administered to said individual locally, e.g., at one or more sites of, or adjacent to, nerve damage that causes said chronic pain, e.g., neuropathic pain. In certain specific embodiments, the composition is administered epicutaneously, subsutaneously, intradermally, subdermally, intramuscularly, intranasally, intrathecally, intraperitoneally, intraosseously, intravesically, epidurally, intracerebrally, intracerebroventricularly, or the like. In certain specific embodiments, the composition is administered locally within 0.5, 1.0, 2.0, 3.0, 4.0 or 5.0 cm from the site of an injury that causes or is associated with neuropathic pain, or from the site of nerve injury that causes or is associated with neuropathic pain. In certain other specific embodiments, the composition is administered locally within 0.5, 1.0, 2.0, 3.0, 4.0 or 5.0 mc from the site of perceived pain, e.g., that area or areas on the individual's body in which the individual perceived the neuropathic pain.

The composition can be, for example, administered locally, distally from a site of neuropathic pain, to a nerve or set of nerves that serve a damaged area of the body of an individual, e.g., an area of the body in which the individual is experiencing the neuropathic pain. For example, the composition can be administered along the spine at any point at which nerve trunks emerge from the spinal column, e.g., any of the cervical nerves, thoracic nerves, or lumbar nerves. In specific embodiments, the composition can be administered adjacent to the spinal cord at which point nerves emerging at C1, C2, C3, C4, C5, C6, or C7, or T1, T2, T3, T4, T5, T6, T7, T8, T9, T10, T11 or T12, or L1, L2, L3, L4 or L5, or at the sacrum.

5.4.4 Treatment of Orthopedic Defects

In another specific embodiment of the methods of treatment described herein, a composition comprising amnion derived adherent cells and platelet-rich plasma is used for the treatment of orthopedic defects, including but not limited to, bone defects, disc herniation and degenerative disc disease.

In a particular aspect, provided herein is a method for treating a bone defect in a subject, comprising administering to a subject in need thereof a therapeutically effective amount of an implantable or injectable composition comprising amnion derived adherent cells and platelet-rich plasma sufficient to treat the bone defect in the subject. In certain embodiments, the bone defect is an osteolytic lesion associated with a cancer, a bone fracture, or a spine, e.g., in need of fusion. In certain embodiments, the osteolytic lesion is associated with multiple myeloma, bone cancer, or metastatic cancer. In certain embodiments, the bone fracture is a non-union fracture. In certain embodiments, an implantable composition comprising amnion derived adherent cells and platelet-rich plasma is administered to the subject. In certain embodiments, an implantable composition is surgically implanted, e.g., at the site of the bone defect. In certain embodiments, an injectable composition comprising amnion derived adherent cells and platelet-rich plasma is administered to the subject. In certain embodiments, an injectable composition is surgically administered to the region of the bone defect.

5.4.4.1 Disc Herniation and Degenerative Disc Disease

In particular, the compositions comprising amnion derived adherent cells and platelet-rich plasma described herein have enhanced utility in the treatment of herniated discs and degenerative disc disease. In some embodiments, the degenerative disc disease is characterized on x-ray tests or MRI scanning of the spine as a narrowing of the normal “disc space” between the adjacent vertebrae.

Disc degeneration, medically referred to as spondylosis, can occur with age when the water and protein content of the cartilage of the body changes. This change results in weaker, more fragile and thin cartilage. Because both the discs and the joints that stack the vertebrae (facet joints) are partly composed of cartilage, these areas are subject to degenerative changes, which renders the disc tissue susceptible to herniation. The gradual deterioration of the disc between the vertebrae is referred to as degenerative disc disease. Degeneration of the disc can cause local pain in the affected area, for example, radiculopathy, i.e., nerve irritation caused by damage to the disc between the vertebrae. In particular, weakness of the outer ring leads to disc bulging and herniation. As a result, the central softer portion of the disc can rupture through the outer ring of the disc and abut the spinal cord or its nerves as they exit the bony spinal column.

Any level of the spine can be affected by disc degeneration. Thus, in some embodiments, the degenerative disc disease treatable by the methods provided herein is cervical disc disease, i.e., disc degeneration that affects the spine of the neck, often accompanied by painful burning or tingling sensations in the arms. In some embodiments, the degenerative disc disease is thoracic disc disease, i.e., disc degeneration that affects the mid-back. In some embodiments, the degenerative disc disease is lumbago, i.e., disc degeneration that affects the lumbar spine.

In particular embodiments, the method for treating degenerative disc disease in a subject comprises administering to a subject in need thereof a therapeutically effective amount of an implantable or injectable composition comprising amnion derived adherent cells and platelet-rich plasma sufficient to treat cervical or lumbar radiculopathy in the subject. In some embodiments, the lumbar radiculopathy is accompanied by incontinence of the bladder and/or bowels. In some embodiments, the method for treating degenerative disc disease in a subject comprises administering to a subject in need thereof a therapeutically effective amount of an implantable or injectable composition comprising amnion derived adherent cells and platelet-rich plasma sufficient to relieve sciatic pain in the subject.

In some embodiments of the methods of treating disc degeneration in an individual with a composition comprising amnion derived adherent cells and platelet rich plasma, as provided herein, disc degeneration of the individual occurs between C1 and C2; between C2 and C3; between C3 and C4; between C4 and C5; between C5 and C6; between C6 and C7; between C7 and T1; between T1 and T2; between T2 and T3; between T3 and T4; between T4 and T5; between T5 and T6; between T6 and T7; between T7 and T8; between T8 and T9; between T9 and T10; between T10 and T11; between T11 and T12; between T12 and L1; between L1 and L2; between L2 and L3; between L3 and L4; or between L4 and L5.

In some embodiments of the methods of treating disc herniation in an individual with a composition comprising amnion derived adherent cells and platelet rich plasma, as provided herein, the disc herniation occurs between C1 and C2; between C2 and C3; Between C3 and C4; between C4 and C5; between C5 and C6; between C6 and C7; between C7 and T1; between T1 and T2; between T2 and T3; between T3 and T4; between T4 and T5; between T5 and T6; between T6 and T7; between T7 and T8; between T8 and T9; between T9 and T10; between T10 and T11; between T11 and T12; between T12 and L1; between L1 and L2; between L2 and L3; between L3 and L4; or between L4 and L5.

Degenerative arthritis (osteoarthritis) of the facet joints is also a cause of localized lumbar pain that can be detected with plain x-ray testing. Wear of the facet cartilage and the bony changes of the adjacent joint is referred to as degenerative facet joint disease or osteoarthritis of the spine.

The methods for treating degenerative disc disease provided herein further encompass treating degenerative disc disease by administering a therapeutically effective amount of a composition comprising amnion derived adherent cells and platelet rich plasma, in conjunction with one or more therapies or treatments used in the course of treating degenerative disc disease. The one or more additional therapies may be used prior to, concurrent with, or after administration of the composition comprising amnion derived adherent cells and platelet rich plasma. In some embodiments, the one or more additional therapies comprise administration of medications to relieve pain and muscles spasm, cortisone injection around the spinal cord (epidural injection), physical therapy (heat, massage, ultrasound, electrical stimulation), and rest (not strict bed rest, but avoiding re-injury).

In some embodiments, the one or more additional therapies comprise operative intervention, for example, where the subject presents with unrelenting pain, severe impairment of function, or incontinence (which can indicate spinal cord irritation). In some embodiments, the operative intervention comprises removal of the herniated disc with laminotomy (producing a small hole in the bone of the spine surrounding the spinal cord), laminectomy (removal of the bony wall adjacent to the nerve tissues), by needle technique through the skin (percutaneous discectomy), disc-dissolving procedures (chemonucleolysis), and others.

5.4.5 Second Therapeutic Compositions and Second Therapies

In any of the above methods of treatment, the method can comprise the administration of a second therapeutic composition or second therapy. The recitation of specific second therapeutic compounds or second therapies in the methods of treating specific diseases, above, are not intended to be exclusive. For example, any of the diseases, disorders or conditions discussed herein can be treated with any of the anti-inflammatory compounds or immunosuppressive compounds described herein. In embodiments in which amnion derived adherent cells are administered with a second therapeutic agent, or with a second type of stem cell, the amnion derived adherent cells and second therapeutic agent and/or second type of stem cell can be administered at the same time or different times, e.g., the administrations can take place within 1, 2, 3, 4, 5, 6, 7, 8, 9 10, 20, 30, 40, or 50 minutes of each other, or 1, 2, 3, 4, 5, 6, 7, 8, 9, 10, 12, 14, 16, 18, 20, or 22 hours of each other, or within 1, 2, 3, 4, 5, 6, 7, 8, 9 or 10 or more days of each other.

In a specific embodiment, treatment of a disease, disorder or condition related to or caused by an inappropriate, deleterious or harmful immune response comprises administration of a second type of stem cell, or population of a second type of stem cell, in addition to the amnion derived adherent cells. In a specific embodiment, said second type of stem cell is a mesenchymal stem cell, e.g., a bone marrow-derived mesenchymal stem cell. In another embodiment, the second type of stem cell is an adipose-derived stem cell. In other embodiments, the second type of stem cell is a multipotent stem cell, a pluripotent stem cell, a progenitor cell, a hematopoietic stem cell, e.g., a CD34⁺ hematopoietic stem cell, an adult stem cell, an embryonic stem cell or an embryonic germ cell. The second type of stem cell, e.g., mesenchymal stem cell or adipose-derived stem cell, can be administered with the amnion derived adherent cells in any ratio, e.g., a ratio of about 100:1, 75:1, 50:1, 25:1, 20:1, 15:1, 10:1, 5:1, 1:1, 1:5, 1:10, 1:15, 1:20, 1:25, 1:50, 1:75 or 1:100. Mesenchymal stem cells can be obtained commercially or from an original source, e.g., bone marrow, bone marrow aspirate, adipose tissue, and the like.

In another specific embodiment, said second therapy comprises an immunomodulatory compound, wherein the immunomodulatory compound is 3-(4-amino-1-oxo-1,3-dihydroisoindol-2-yl)-piperidine-2,6-dione; 3-(4′aminoisolindoline-1′-onw)-1-piperidine-2,6-dione; 4-(Amino)-2-(2,6-dioxo(3-piperidyl))-isoindoline-1,3-dione; or α-(3-aminophthalimido) glutarimide. In a more specific embodiment, said immunomodulatory compound is a compound having the structure

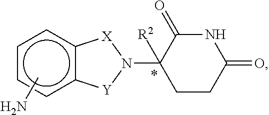

wherein one of X and Y is C═O, the other of X and Y is C═O or CH₂, and R² is hydrogen or lower alkyl, or a pharmaceutically acceptable salt, hydrate, solvate, clathrate, enantiomer, diastereomer, racemate, or mixture of stereoisomers thereof. In another more specific embodiment, said immunomodulatory compound is a compound having the structure

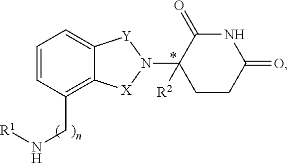

wherein one of X and Y is C═O and the other is CH₂ or C═O;

R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;

R² is H, F, benzyl, (C₁-C₈)alkyl, (C₂-C₈)alkenyl, or (C₂-C₈)alkynyl;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl;

each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;

n is 0 or 1; and

* represents a chiral-carbon center;

or a pharmaceutically acceptable salt, hydrate, solvate, clathrate, enantiomer, diastereomer, racemate, or mixture of stereoisomers thereof. In another more specific embodiment, said immunomodulatory compound is a compound having the structure

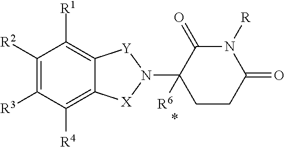

wherein:

one of X and Y is C═O and the other is CH₂ or C═O;

R is H or CH₂OCOR′;

(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR³ and the remaining of R¹, R², R³, or R⁴ are hydrogen;

R⁵ is hydrogen or alkyl of 1 to 8 carbons

R⁶ hydrogen, alkyl of 1 to 8 carbon atoms, benzo, chloro, or fluoro;

R′ is R⁷—CHR¹⁰—N(R⁸R⁹);

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;

R¹⁰ is hydrogen, alkyl of to 8 carbon atoms, or phenyl; and

* represents a chiral-carbon center;

or a pharmaceutically acceptable salt, hydrate, solvate, clathrate, enantiomer, diastereomer, racemate, or mixture of stereoisomers thereof.

Any combination of the above therapeutic agents can be administered. Such therapeutic agents can be administered in any combination with the amnion derived adherent cells, at the same time or as a separate course of treatment.

Amnion derived adherent cells can be administered, to the individual suffering from an immune-related disease, in the form of a pharmaceutical composition, e.g., a pharmaceutical composition suitable for intravenous, intramuscular or intraperitoneal injection. Amnion derived adherent cells can be administered in a single dose, or in multiple doses. Where amnion derived adherent cells are administered in multiple doses, the doses can be part of a therapeutic regimen designed to relieve one or more acute symptoms of an immune-related disease or disorder, e.g., IBD, e.g., Crohn's disease, of can be part of a long-term therapeutic regimen designed to prevent, or lessen the severity, of, e.g., a chronic course of the disease. In embodiments in which amnion derived adherent cells are administered with a second therapeutic agent, or with a second type of stem cell, the amnion derived adherent cells and second therapeutic agent and/or second type of stem cell can be administered at the same time or different times, e.g., the administrations can take place within 1, 2, 3, 4, 5, 6, 7, 8, 9 10, 20, 30, 40, or 50 minutes of each other, or 1, 2, 3, 4, 5, 6, 7, 8, 9, 10, 12, 14, 16, 18, 20, or 22 hours of each other, or within 1, 2, 3, 4, 5, 6, 7 8, 9 or 10 or more days of each other.

### 5.5 Amnion Derived Adherent Cells

The methods provided herein use tissue culture plastic adherent, amnion derived cells, and populations of such cells, referred to herein as “amnion derived adherent cells” or AMDACs. Generally, amnion derived adherent cells superficially resemble fibroblasts or mesenchymal cells in appearance, having a generally fibroblastoid shape. Such cells adhere to a cell culture surface, e.g., to tissue culture plastic. In certain embodiments of any of the AMDACs disclosed herein, the cells are human cells.

AMDACs provided herein display cellular markers that distinguish them from other amnion-derived, or placenta-derived, cells. In certain embodiments of each of the embodiments of AMDACs described herein, the AMDACs are isolated.

In one embodiment, amnion derived adherent cells are OCT-4⁻ (octamer binding protein 4), as determinable by RT-PCR. In another specific embodiment, OCT-4⁻ amnion derived adherent cells are CD49f⁺, as determinable, e.g., by immunolocalization, e.g., flow cytometry. In another specific embodiment, said OCT-4⁻ cells are HLA-G⁻, as determinable by RT-PCR. In another specific embodiment, the OCT-4⁻ cells are VEGFR1/Flt-1⁺ (vascular endothelial growth factor receptor 1) and/or VEGFR2/KDR⁺ (vascular endothelial growth factor receptor 2), as determinable by immunolocalization, e.g., flow cytometry. In a specific embodiment, OCT-4⁻ amnion derived adherent cells express at least 2 log less PCR-amplified mRNA for OCT-4 at, e.g., 20 cycles, than an equivalent number of NTERA-2 cells and RNA amplification cycles. In another specific embodiment, said OCT-4⁻ cells are CD90⁺, CD105⁺, or CD117⁻ as determinable, e.g., by immunolocalization, e.g., flow cytometry. In a more specific embodiment, said OCT-4⁻ cells are CD90⁺, CD105⁺, and CD117⁻ as determinable, e.g., by immunolocalization, e.g., flow cytometry. In a more specific embodiment, the cells are OCT-4⁻ or HLA-G⁻, and is additionally CD49f⁺, CD90⁺, CD105⁺, and CD117⁻ as determinable, e.g., by immunolocalization, e.g., flow cytometry. In a more specific embodiment, the cells are OCT-4⁻, HLA-G⁻, CD49f⁺, CD90⁺, CD105⁺, and CD117⁻ as determinable, e.g. by immunolocalization, e.g., flow cytometry. In another specific embodiment, the OCT-4⁻ cells do not express SOX2, e.g., as determinable by RT-PCR for 30 cycles. In a specific embodiment, therefore, the amnion derived adherent cells are OCT-4⁻, CD49f⁺, CD90⁺. CD105⁺, and CD117⁻, as determinable by immunolocalization, e.g., flow cytometry, and SOX2⁻, as determinable by RT-PCR, e.g., for 30 cycles.

In a specific embodiment, the AMDACs described herein are GFAP⁺ as determinable by, e.g., a short-term neural differentiation assay (See, e.g., Section 6.3.3, below). In another specific embodiment, the AMDACs described herein are beta-tubulin III (Tuj1)⁺ as determinable by, e.g., a short-term neural differentiation assay. In another specific embodiment, the AMDACs are OCT-4⁻, GFAP⁺, and beta-tubulin III (Tuj1)⁺. In another specific embodiment, the AMDACs are OCT-4⁻, CD200⁺, CD105⁺, and CD49f⁺. In another specific embodiment, the AMDACs are CD200⁺, CD105⁺, CD90⁺, and CD73⁺. In another specific embodiment, the AMDACs described herein are CD117⁻ and are not selected using an antibody to CD117. In another specific embodiment, the AMDACs described herein are CD146⁻ and are not selected using an antibody to CD146. In another specific embodiment, the AMDACs described herein OCT-4⁻, as determinable by RT-PCR and/or immunolocalization, e.g., flow cytometry, and do not express CD34 following induction with VEGF, e.g., as determinable by RT-PCR and/or immunolocalization, e.g., flow cytometry. In another specific embodiment, the AMDACs used in the methods described herein are neurogenic, as determinable by a short-term neural differentiation assay (see, e.g., Section 6.3.3, below). In another specific embodiment, the AMDACs used in the methods described herein are non-chondrogenic as determinable by an in vitro chondrogenic potential assay (see, e.g., Section 6.3.2, below). In another specific embodiment, the AMDACs used in the methods described herein are non-osteogenic as determinable by an osteogenic phenotype assay (see, e.g., Section 6.3.1, below). In another specific embodiment, the AMDACs described herein are non-osteogenic after being cultured for up to 6 weeks (e.g., for 2 weeks, for 4 weeks, or for 6 weeks) in DMEM at pH 7.4 (High glucose) supplemented with 100 nM dexamethasone, 10 mM (3-glycerol phosphate, 50 μM L-ascorbic acid-2-phosphate, wherein osteogenesis is assessable using von Kossa staining; alizarin red staining; or detectable by the presence of osteopontin, osteocalcin, osteonectin, and/or bone sialoprotein by, e.g., RT-PCR.

In another embodiment, said OCT-4⁻ cells are one or more of CD29⁺, CD73⁺, ABC-p⁺, and CD38⁻, e.g., as determinable by immunolocalization, e.g., flow cytometry.

In another specific embodiment, for example, OCT-4⁻ AMDACs can additionally be one or more of CD9⁺, CD10⁺, CD44⁺, CD54⁺, CD98⁻, TEM-7⁺ (tumor endothelial marker 7), CD31⁻, CD34⁻, CD45⁻, CD133⁻, CD143⁻ (angiotensin-1-converting enzyme, ACE), CD146⁻ (melanoma cell adhesion molecule), or CXCR4⁻ (chemokine (C-X-C motif) receptor 4), e.g., as determinable by immunolocalization, e.g., flow cytometry, or HLA-G⁻ as determinable by RT-PCR. In a more specific embodiment, said cells are CD9⁺, CD10⁻, CD44⁺, CD54⁺, CD98⁻, Tie-2⁺, TEM-7′, CD31⁻, CD34⁻, CD45⁻, CD133⁻, CD143⁻, CD146⁻, and CXCR4⁻, e.g., as determinable by immunolocalization, e.g., flow cytometry, and HLA-G⁻ as determinable by RT-PCR. In another embodiment, the amnion derived adherent cells are one or more of CD31⁻, CD34⁻, CD45⁻, and/or CD133⁻, as determinable, e.g., by immunolocalization, e.g., flow cytometry. In a specific embodiment, the amnion derived adherent cells are OCT-4⁻, as determinable by RT-PCR; VEGFR1/Flt-1⁺ and/or VEGFR2/KDR⁺, as determinable by immunolocalization, e.g., flow cytometry; and one or more, or all, of CD31⁻, CD34⁻, CD45⁻, and/or CD133⁻ as determinable, e.g., by immunolocalization, e.g., flow cytometry.

In another specific embodiment, said AMDACs are additionally VE-cadherin⁻ as determinable by immunolocalization, e.g., flow cytometry. In another specific embodiment, said OCT-4⁻ cells are, either alone or in combination with other markers, additionally positive for CD105⁺ and CD200⁺ as determinable by immunolocalization, e.g., flow cytometry. In another specific embodiment, said cells do not express CD34 as detected by immunolocalization, e.g., flow cytometry, after exposure to 1 to 100 ng/mL VEGF for 4 to 21 days. In more specific embodiments, said cells do not express CD34 as detected by immunolocalization, e.g., flow cytometry, after exposure to 25 to 75 ng/mL VEGF for 4 to 21 days, or to 50 ng/mL VEGF for 4 to 21 days. In even more specific embodiments, said cells do not express CD34 as detected by immunolocalization, e.g., flow cytometry, after exposure to 1, 2.5, 5, 10, 25, 50, 75 or 100 ng/mL VEGF for 4 to 21 days. In yet more specific embodiments, said cells do not express CD34 as detected by immunolocalization, e.g., flow cytometry, after exposure to 1 to 100 ng/mL VEGF for 7 to 14, e.g., 7, days.

In specific embodiments, the amnion derived adherent cells are OCT-4⁻, as determinable by RT-PCR, and one or more of VE-cadherin⁻, VEGFR2/KDR⁺, CD9⁺, CD54⁺, CD105⁺, and/or CD200⁺ as determinable by immunolocalization, e.g., flow cytometry. In a specific embodiment, the amnion derived adherent cells are OCT-4⁻, as determinable by RT-PCR, and VE-cadherin⁻, VEGFR2/KDR⁺, CD9⁺, CD54⁺, CD105⁺, and, CD200⁺ as determinable by immunolocalization, e.g., flow cytometry. In another specific embodiment, said cells do not express CD34, as detected by immunolocalization, e.g., flow cytometry, e.g., after exposure to 1 to 100 ng/mL VEGF for 4 to 21 days.

In another embodiment, the amnion derived adherent cells are OCT-4⁻, CD49f⁺, HLA-G⁻, CD90⁺, CD105⁺, and CD117⁻. In a more specific embodiment, said cells are one or more of CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, or CXCR4⁻, as determinable by immunolocalization, e.g., flow cytometry. In a more specific embodiment, said cells CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and CXCR4⁻ as determinable by immunolocalization. e.g., flow cytometry. In another specific embodiment, said cells are additionally VEGFR1/Flt-1⁺ and/or VEGFR2/KDR⁺, as determinable by immunolocalization, e.g., flow cytometry; and one or more of CD31⁻, CD34⁻, CD45⁻, CD133⁻, and/or Tie-2⁻ as determinable by immunolocalization, e.g., flow cytometry. In another specific embodiment, said cells are additionally VEGFR1/Flt-1⁺, VEGFR2/KDR⁺, CD31⁻, CD34⁻, CD45⁻, CD133⁻, and Tie-2⁻ as determinable by immunolocalization, e.g., flow cytometry.

In another embodiment, the OCT-4-amnion derived adherent cells are additionally one or more, or all, of CD9⁺, CD10⁺, CD44⁺, CD49f⁺, CD54⁺, CD90⁺, CD98⁺, CD105⁺, CD200⁺, Tie-2⁺, TEM-7⁺, VEGFR1/Flt-1⁺, and/or VEGFR2/KDR⁺ (CD309⁺), as determinable by immunolocalization, e.g., flow cytometry; or additionally one or more, or all, of CD31⁻, CD34⁻, CD38⁻, CD45⁻, CD117⁻, CD133⁻, CD143⁻, CD144⁻, CD146⁻, CD271⁻, CXCR4⁻, HLA-G⁻, and/or VE-cadherin⁻, as determinable by immunolocalization, e.g., flow cytometry, or SOX2⁻, as determinable by RT-PCR.

In certain embodiments, the isolated tissue culture plastic-adherent amnion derived adherent cells are CD49f⁺. In a specific embodiment, said CD49f⁺ cells are additionally one or more, or all, of CD9⁺, CD10⁺, CD44⁺, CD54⁺, CD90⁺, CD98⁺, CD105⁺, CD200⁺, Tie-2⁺, TEM-7⁺, VEGFR1/Flt-1⁺, and/or VEGFR2/KDR⁺ (CD309⁺), as determinable by immunolocalization, e.g., flow cytometry; or additionally one or more, or all, of CD31⁻, CD34⁻, CD38⁻, CD45⁻, CD117⁻, CD133⁻, CD143⁻, CD144⁻, CD146⁻, CD271⁻, CXCR4⁻, HLA-G⁻, OCT-4⁻ and/or VE-cadherin⁻, as determinable by immunolocalization, e.g., flow cytometry, or SOX2⁻, as determinable by RT-PCR.

In certain other embodiments, the isolated tissue culture plastic-adherent amnion derived adherent cells are HLA-G⁻, CD90⁺, and CD117⁻. In a specific embodiment, said HLA-G⁻, CD90⁺, and CD117⁻ cells are additionally one or more, or all, of CD9⁺, CD10⁺, CD44⁺, CD49f⁺, CD54⁻, CD98⁺, CD105⁺, CD200⁺, Tie-2⁺, TEM-7⁺, VEGFR1/Flt-1⁺, and/or VEGFR2/KDR⁺ (CD309⁺), as determinable by immunolocalization, e.g., flow cytometry; or additionally one or more, or all, of CD31⁻, CD34⁻, CD38⁻, CD45⁻, CD133⁻, CD143⁻, CD144⁻, CD146⁻, CD271⁻, CXCR4⁻, OCT-4⁻ and/or VE-cadherin⁻, as determinable by immunolocalization, e.g., flow cytometry, or SOX2⁻, as determinable by RT-PCR.

In another embodiment, the isolated amnion derived adherent cells do not constitutively express mRNA for fibroblast growth factor 4 (FGF4), interferon γ (IFNG), chemokine (C-X-C motif) ligand 10 (CXCL10), angiopoietin 4 (ANGPT4), angiopoietin-like 3 (ANGPTL3), fibrinogen a chain (FGA), leptin (LEP), prolactin (PRL), prokineticin 1 (PROK1), tenomodulin (TNMD), FMS-like tyrosine kinase 3 (FLT3), extracellular link domain containing 1 (XLKD1), cadherin 5, type 2 (CDH5), leukocyte cell derived chemotaxin 1 (LECT1), plasminogen (PLG), telomerase reverse transcriptase (TERT), (sex determining region Y)-box 2 (SOX2), NANOG, matrix metalloprotease 13 (MMP-13), distal-less homeobox 5 (DLX5), and/or bone gamma-carboxyglutamate (gla) protein (BGLAP), as determinable by RT-PCR, e.g., for 30 cycles under standard culture conditions. In other embodiments, isolated amnion derived adherent cells, or population of amnion derived adherent cells, express mRNA for (ARNT2), nerve growth factor (NGF), brain-derived neurotrophic factor (BDNF), glial-derived neurotrophic factor (GDNF), neurotrophin 3 (NT-3), NT-5, hypoxia-Inducible Factor 1α (HIF1A), hypoxia-inducible protein 2 (HIG2), heme oxygenase (decycling) 1 (HMOX1), Extracellular superoxide dismutase [Cu—Zn] (SOD3), catalase (CAT), transforming growth factor β1 (TGFB1), transforming growth factor β1 receptor (TGFB1R), and hepatoycte growth factor receptor (HGFR/c-met)

In another aspect, provided herein are isolated populations of cells, e.g., isolated populations of amnion cells or placental cells, or substantially isolated populations of AMDACs, comprising the amnion derived adherent cells described herein. The populations of cells can be homogeneous populations, e.g., a population of cells, at least about 90%, 95%, 98% or 99% of which are amnion derived adherent cells. The populations of cells can be heterogeneous, e.g., a population of cells wherein at most about 10%, 20%, 30%, 40%, 50%, 60%, 70% or 80% of the cells in the population are amnion derived adherent cells. The isolated populations of cells are not, however, tissue, i.e., amniotic membrane.

In one embodiment, provided herein is an isolated population of cells comprising AMDACs, e.g., a population of cells substantially homogeneous for AMDACs, or a population of cells heterogeneous with respect to the AMDACs, wherein said AMDACs are adherent to tissue culture plastic, and wherein said AMDACs are OCT-4⁻, as determinable by RT-PCR. In a specific embodiment, the AMDACs are CD49f⁺ or HLA-G⁺, e.g., as determinable by immunolocalization, e.g., flow cytometry, or RT-PCR. In another specific embodiment, said AMDACs in said population of cells are VEGFR1/Flt-1⁺ and/or VEGFR2/KDR⁺ as determinable by immunolocalization, e.g., flow cytometry, wherein said isolated population of cells is not an amnion or amniotic membrane or other tissue. In a more specific embodiment, the AMDACs in said population of cells are OCT-4⁻, and/or HLA-G⁻ as determinable by RT-PCR, and VEGFR1/Flt-1⁺ and/or VEGFR2/KDR⁺ as determinable by immunolocalization. e.g., flow cytometry. In another specific embodiment, said AMDACs are CD90⁺, CD105⁺, or CD117⁻. In a more specific embodiment, said AMDACs are CD90⁺, CD105⁺, and CD117⁻. In a more specific embodiment, the AMDACs are OCT-4⁻, CD49f⁺, CD90⁺, CD105⁺, and CD117⁻. In another specific embodiment, the AMDACs do not express SOX2, e.g., as determinable by RT-PCR for 30 cycles. In an even more specific embodiment, the population comprises AMDACs, wherein said AMDACs are OCT-4⁻, HLA-G⁻, CD49f⁺, CD90⁺, CD105⁺, and CD117⁻, as determinable by immunolocalization, e.g., flow cytometry, and SOX2⁻, e.g., as determinable by RT-PCR for 30 cycles.

In another specific embodiment, said AMDACs in said population of cells are CD90⁺, CD105⁺, or CD117⁻, as determinable by immunolocalization, e.g., flow cytometry. In a more specific embodiment, the AMDACs are CD90⁺, CD105⁺, and CD117⁻, as determinable by immunolocalization, e.g., flow cytometry. In a more specific embodiment, the AMDACs are OCT-4⁻ or HLA-G⁻, e.g., as determinable by RT-PCR, and are additionally CD49f⁺, CD90⁺, CD105⁺, and CD117⁻ as determinable by immunolocalization, e.g., flow cytometry. In a more specific embodiment, the AMDACs in said population of cells are OCT-4⁻, HLA-G⁻, CD49f⁺, CD90⁺, CD105⁺, and CD117⁻. In another specific embodiment, the AMDACs do not express SOX2, e.g., as determinable by RT-PCR for 30 cycles. In a more specific embodiment, therefore, the AMDACs are OCT-4⁻, CD49f⁺, CD90⁺, CD105⁺, and CD117⁻, as determinable by immunolocalization, e.g., flow cytometry, and SOX2⁻, as determinable by RT-PCR, e.g., for 30 cycles. In an even more specific embodiment, the AMDACs are OCT-4⁻ or HLA-G⁻, and are additionally CD49f⁺, CD90⁺, CD105⁺, and CD117⁻. In a more specific embodiment, the AMDACs are OCT-4⁻, HLA-G⁻, CD49f⁺, CD90⁺, CD105⁺, and CD117⁻.

In another embodiment, the amnion derived adherent cells in said population of cells are adherent to tissue culture plastic, OCT-4⁻ as determinable by RT-PCR, and VEGFR1/Flt-1⁺ and/or VEGFR2/KDR⁺ as determinable by immunolocalization, e.g., flow cytometry, and are additionally one or more of CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, or CXCR4⁻, as determinable by mmunolocalization, e.g., flow cytometry, or HLA-G⁻ as determinable by RT-PCR, and wherein said isolated population of cells is not an amnion. In another embodiment, provided herein is an isolated population of cells comprising amnion derived adherent cells, wherein said cells are adherent to tissue culture plastic, wherein said cells are OCT-4⁻ as determinable by RT-PCR, and VEGFR1/Flt-1⁺ and/or VEGFR2/KDR⁺ as determinable by immunolocalization, e.g., flow cytometry, wherein said cells do not express CD34 as detected by immunolocalization, e.g. flow cytometry, after exposure to 1 to 100 ng/mL VEGF for 4 to 21 days, and wherein said isolated population of cells is not an amnion.

In a specific embodiment of any of the above embodiments, at least about 50%, 60%, 70%, 80%, 90%, 95%, 98% or 99% of cells in said population are said amnion derived adherent cells, as described or characterizable by any of the cellular marker combinations described above.

In another embodiment, any of the above populations of cells comprising amnion derived adherent cells forms sprouts or tube-like structures when cultured in the presence of an extracellular matrix protein, e.g., like collagen type I and IV, or an angiogenic factor, e.g., like vascular endothelial growth factor (VEGF), epithelial growth factor (EGF), platelet derived growth factor (PDGF) or basic fibroblast growth factor (bFGF), e.g., in or on a substrate such as placental collagen, e.g., or MATRIGEL™ for at least 4 days and up to 14 days.

In certain embodiments, provided herein is a cell that expresses, or a population of cells, wherein at least about 50%, 60%, 70%, 80%, 90%, 95% or 98% of cells in said isolated population of cells are amnion derived adherent cells that express RNA for one or more of, or all of, ACTA2 (actin, alpha 2, smooth muscle, aorta), ADAMTS1 (ADAM metallopeptidase with thrombospondin type 1 motif, 1), AMOT (angiomotin), ANG (angiogenin), ANGPT1 (angiopoietin 1), ANGPT2, ANGPTL1 (angiopoietin-like 1), ANGPTL2, ANGPTL4, BAI1 (brain-specific angiogenesis inhibitor 1), CD44, CD200, CEACAM1 (carcinoembryonic antigen-related cell adhesion molecule 1), CHGA (chromogranin A), COL15A1 (collagen, type XV, alpha 1), COL18A1 (collagen, type XVIII, alpha 1), COL4A1 (collagen, type IV, alpha 1), COL4A2 (collagen, type IV, alpha 2), COL4A3 (collagen, type IV, alpha 3), CSF3 (colony stimulating factor 3 (granulocyte), CTGF (connective tissue growth factor), CXCL12 (chemokine (CXC motif) ligand 12 (stromal cell-derived factor 1)), CXCL2, DNMT3B (DNA (cytosine-5-)-methyltransferase 3 beta), ECGF1 (thymidine phosphorylase), EDG1 (endothelial cell differentiation gene 1), EDIL3 (EGF-like repeats and discoidin I-like domains 3), ENPP2 (ectonucleotide pyrophosphatase/phosphodiesterase 2), EPHB2 (EPH receptor B2), FBLN5 (F1BULIN 5), F2 (coagulation factor II (thrombin)), FGF1 (acidic fibroblast growth factor), FGF2 (basic fibroblast growth factor), FIGF (c-fos induced growth factor (vascular endothelial growth factor D)), FLT4 (fms-related tyrosine kinase 4), FN1 (fibronectin 1), FST (follistatin), FOXC2 (forkhead box C2 (MFH-1, mesenchyme forkhead 1)), GRN (granulin), HGF (hepatocyte growth factor), HEY1 (hairy/enhancer-of-split related with YRPW motif 1). HSPG2 (heparan sulfate proteoglycan 2), IFNB1 (interferon, beta 1, fibroblast), IL8 (interleukin 8), IL12A, ITGA4 (integrin, alpha 4; CD49d), ITGAV (integrin, alpha V), ITGB3 (integrin, beta 3), MDK (midkine), MMP2 (matrix metalloprotease 2), MYOZ2 (myozenin 2), NRP1 (neuropilin 1), NRP2, PDGFB (platelet-derived growth factor β), PDGFRA (platelet-derived growth factor receptor α), PDGFRB, PECAM1 (platelet/endothelial cell adhesion molecule), PF4 (platelet factor 4), PGK1 (phosphoglycerate kinase 1), PROX1 (prospero homeobox 1), PTN (pleiotrophin), SEMA3F (semophorin 3F), SERPINB5 (serpin peptidase inhibitor, clade B (ovalbumin), member 5), SERPINC1, SEROINF1, TIMP2 (tissue inhibitor of metalloproteinases 2), TIMP3, TGFA (transforming growth factor, alpha), TGFB1, THBS1 (thrombospondin 1), THBS2, TIE1 (tyrosine kinase with immunoglobulin-like and EGF-like domains 1), TIE2/TEK, TNF (tumor necrosis factor), TNNI1 (troponin I, type 1), TNFSF15 (tumor necrosis factor (ligand) superfamily, member 15), VASH1 (vasohibin 1), VEGF (vascular endothelial growth factor), VEGFB, VEGFC, VEGFR1/FLT1 (vascular endothelial growth factor receptor 1), and/or VEGFR2/KDR.

When human cells are used, the gene designations throughout refer to human sequences, and, as is well known to persons of skill in the art, representative sequences can be found in literature, or in GenBank. Probes to the sequences can be determined by sequences that are publicly-available, or through commercial sources, e.g., specific TAQMAN® probes or TAQMAN® Angiogenesis Array (Applied Biosystems, part no. 4378710).

In certain embodiments, provided herein is a cell that expresses, or a population of cells, wherein at least about 50%, 60%, 70%, 80%, 90%, 95% or 98% of cells in said isolated population of cells are amnion derived adherent cells that express CD49d, Connexin-43, HLA-ABC, Beta 2-microglobulin, CD349, CD318, PDLL, CD106, Galectin-1, ADAM 17 precursor (A disintegrin and metalloproteinase domain 17) (TNF-alpha converting enzyme) (TNF-alpha convertase), Angiotensinogen precursor, Filamin A (Alpha-filamin) (Filamin 1) (Endothelial actin-binding protein) (ABP-280) (Nonmuscle filamin), Alpha-actinin 1 (Alpha-actinin cytoskeletal isoform) (Non-muscle alpha-actinin 1) (F-actin cross linking protein), Low-density lipoprotein receptor-related protein 2 precursor (Megalin) (Glycoprotein 330) (gp330), Macrophage scavenger receptor types I and II (Macrophage acetylated LDL receptor I and II), Activin receptor type IIB precursor (ACTR-IIB), Wnt-9 protein, Glial fibrillary acidic protein, astrocyte (GFAP), Myosin-binding protein C, cardiac-type (Cardiac MyBP-C) (C-protein, cardiac muscle isoform), and/or Myosin heavy chain, nonmuscle type A (Cellular myosin heavy chain, type A) (Nonmuscle myosin heavy chain-A) (NMMHC-A), as detectable by immunolocalization.

In certain embodiments, the amnion derived adherent cells, or population of cells comprising amnion derived adherent cells, e.g., wherein at least about 50%, 60%, 70%, 80%, 90%, 95% or 98% of cells in said isolated population of cells are amnion derived adherent cells, secrete one or more, or all, of VEGF, HGF, IL-8, MCP-3, FGF2, Follistatin, G-CSF, EGF, ENA-78, GRO, IL-6, MCP-1, PDGF-BB, TIMP-2, uPAR, Galectin-1, e.g., into culture medium in which the cell, or cells, are grown.

In another embodiment, provided herein is a population of cells, e.g., a population of amnion derived adherent cells, or a population of cells wherein at least about 50%, 60%, 70%, 80%, 90%, 95% or 98% of cells in said isolated population of cells are amnion derived adherent cells that express micro RNAs (miRNAs) at a higher level than bone marrow-derived mesenchymal stem cells, wherein said miRNAs comprise one or more, or all of, miR-17-3p, miR-18a, miR-18b, miR-19b, miR-92, and/or miR-296. In another embodiment, provided herein is a population of cells, e.g., a population of amnion derived adherent cells, or a population of cells wherein at least about 50%, 60%, 70%, 80%, 90%, 95% or 98% of cells in said isolated population of cells are amnion derived adherent cells that express one or more of, or all of, micro RNAs (miRNAs) at a lower level than bone marrow-derived mesenchymal stem cells, wherein said miRNAs comprise one or more, or all of, miR-20a, miR-20b, miR-221, miR-222, miR-15b, and/or miR-16. In certain embodiments, AMDACs, or populations of AMDACs, express one or more, or all, of the angiogenic miRNAs miR-17-3p, miR-18a, miR-18b, miR-19b, miR-92, miR-20a, miR-20b, (members of the of the angiogenic miRNA cluster 17-92), miR-296, miR-221, miR-222, miR-15b, and/or miR-16.

In one embodiment, provided herein are isolated amnion derived adherent cells, wherein said cells are adherent to tissue culture plastic, wherein said cells are OCT-4⁻, as determinable by RT-PCR, and CD49f⁺, HLA-G⁻, CD90⁺, CD105⁺, and CD117⁻, as determinable by immunolocalization, e.g., flow cytometry, and wherein said cells: (a) express one or more of CD9, CD10, CD44, CD54, CD98, CD200, Tie-2, TEM-7, VEGFR1/Flt-1, or VEGFR2/KDR (CD309), as determinable by immunolocalization, e.g., flow cytometry; (b) lack expression of CD31, CD34, CD38, CD45, CD133, CD143, CD144, CD146, CD271, CXCR4, HLA-G, or VE-cadherin, as determinable by immunolocalization, e.g., flow cytometry; (c) lack expression of SOX2, as determinable by RT-PCR; (d) express mRNA for ACTA2, ADAMTS1, AMOT, ANG, ANGPT1, ANGPT2, ANGPTL1, ANGPTL2, ANGPTL4, BA11, CD44, CD200, CEACAM1, CHGA, COL15A1, COL18A1, COL4A1, COL4A2, COL4A3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, GRN, HGF, HEY1, HSPG2, IFNB1, IL8, IL12A, ITGA4, ITGAV, ITGB3, MDK, MMP2, MYOZ2, NRP1, NRP2, PDGFB, PDGFRA, PDGFRB, PECAM1, PF4, PGK1, PROX1, PTN, SEMA3F, SERPINB5, SERPINC1, SERPINF1, TIMP2, TIMP3, TGFA, TGFB1, THBS1, THBS2, TIE1, TIE2/TEK, TNF, TNNI1, TNFSF15, VASH1, VEGF, VEGFB, VEGFC, VEGFR1/FLT1, or VEGFR2/KDR; (e) express one or more of the proteins CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type IIB precursor, Wnt-9 protein, glial fibrillary acidic protein, astrocyte, myosin-binding protein C, or myosin heavy chain, nonmuscle type A; (f) secret VEGF, HGF, IL-8, MCP-3, FGF2, Follistatin, G-CSF, EGF, ENA-78, GRO, IL-6, MCP-1, PDGF-BB, TIMP-2, uPAR, or galectin-1 into culture medium in which the cell grows; (g) express micro RNAs miR-17-3p, miR-18a, miR-18b, miR-19b, miR-92, or miR-296 at a higher level than an equivalent number of bone marrow-derived mesenchymal stem cells; (h) express micro RNAs miR-20a, miR-20b, miR-221, miR-222, miR-15b, or miR-16 at a lower level than an equivalent number of bone marrow-derived mesenchymal stem cells; (i) express miRNAs miR-17-3p, miR-18a, miR-18b, miR-19b, miR-92, miR-20a, miR-20b, miR-296, miR-221, miR-222, miR-15b, or miR-16; and/or (j) express increased levels of CD202b, IL-8 or VEGF when cultured in less than about 5% O₂, compared to expression of CD202b, IL-8 or VEGF under 21% O₂. In a specific embodiment, the isolated amnion derived adherent cells are OCT-4⁻, as determinable by RT-PCR, and CD49f⁺, HLA-G⁻, CD90⁺, CD105⁺, and CD117⁻, as determinable by immunolocalization, e.g., flow cytometry, and (a) express CD9, CD10, CD44, CD54, CD90, CD98, CD200, Tie-2, TEM-7, VEGFR1/Flt-1, and VEGFR2/KDR (CD309), as determinable by immunolocalization, e.g. flow cytometry; (b) lack expression of CD31, CD34, CD38, CD45, CD133, CD143. CD144, CD146, CD271, CXCR4, HLA-G, and VE-cadherin, as determinable by immunolocalization, e.g., flow cytometry; (c) lack expression of SOX2, as determinable by RT-PCR; (d) express mRNA for ACTA2, ADAMTS1, AMOT, ANG, ANGPT1, ANGPT2, ANGPTL1, ANGPTL2, ANGPTL4, BAI1, CD44, CD200, CEACAM1, CHGA, COL15A1, COL18A1, COL4A1, COL4A2, COL4A3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, GRN, HGF, HEY1. HSPG2, IFNB1, IL8, IL12A, ITGA4, ITGAV, ITGB3, MDK, MMP2, MYOZ2, NRP1, NRP2, PDGFB, PDGFRA, PDGFRB, PECAM1, PF4, PGK1, PROX1, PTN, SEMA3F, SERPINB5, SERPINC1, SERPINF1, TIMP2, TIMP3, TGFA, TGFB1, THBS1, THBS2, TIE1, TIE2/TEK, TNF, TNNI1, TNFSF15, VASH1, VEGF, VEGFB, VEGFC, VEGFR1/FLT1, and/or VEGFR2/KDR; (e) express one or more of CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type IIB precursor, Wnt-9 protein, glial fibrillary acidic protein, astrocyte, myosin-binding protein C, and/or myosin heavy chain, nonmuscle type A; (f) secrete VEGF, HGF, IL-8, MCP-3, FGF2, Follistatin, G-CSF, EGF, ENA-78, GRO, IL-6, MCP-1, PDGF-BB, TIMP-2, uPAR, and/or Galectin-1, e.g., into culture medium in which the cells grow; (g) express micro RNAs miR-17-3p, miR-18a, miR-18b, miR-19b, miR-92, and miR-296 at a higher level than an equivalent number of bone marrow-derived mesenchymal stem cells; (h) express micro RNAs miR-20a, miR-20b, miR-221, miR-222, miR-15b, and miR-16 at a lower level than an equivalent number of bone marrow-derived mesenchymal stem cells; (i) express miRNAs miR-17-3p, miR-18a, miR-18b, miR-19b, miR-92, miR-20a, miR-20b, miR-296, miR-221, miR-222, miR-15b, and miR-16; and/or (i) expresses increased levels of CD202b, IL-8 and VEGF when cultured in less than about 5% O₂, compared to expression of CD202b, IL-8 and/or VEGF when said cells are cultured under 21% O₂. Further provided herein are populations of cells comprising AMDACs, e.g. populations of AMDACs, having one or more of the above-recited characteristics.

In other specific embodiments, the population of cells comprising amnion-derived angiogenic cells secretes one or more angiogenic factors and thereby induces human endothelial cells to migrate in an in vitro wound healing assay. In other specific embodiments, the population of cells comprising amnion derived adherent cells induces maturation, differentiation or proliferation of human endothelial cells, endothelial progenitors, myocytes or myoblasts.

In another embodiment, any of the above amnion derived adherent cells, or populations of cells comprising amnion derived adherent cells, take up acetylated low density lipoprotein (LDL) when cultured in the presence of extracellular matrix proteins, e.g., collagen type I or IV, and/or one or more angiogenic factors, e.g., VEGF, EGF, PDGF, or bFGF, e.g., on a substrate such as placental collagen or MATRIGEL™.

In another embodiment, the AMDACs are comprised within a population of cells. In specific embodiments of such embodiments, the amnion derived adherent cells are adherent to tissue culture plastic, are OCT-4⁻, as determinable by RT-PCR, and VEGF2/KDR⁺, CD9⁻, CD54⁺, CD105⁺, CD200⁺, or VE-cadherin⁻, as determinable by immunolocalization, e.g., flow cytometry. In specific embodiments, at least 10%, 20%, 30%. 40%, 50%, 60%, 70%, 80%, 90%, 95%, 98% or 99% of the cells in said population of cells are amnion derived cells that are OCT-4⁻, as determinable by RT-PCR, and VEGFR2/KDR⁺, CD9⁺, CD54⁺, CD105⁺, CD200⁺, or VE-cadherin⁻, as determinable by immunolocalization, e.g., flow cytometry. In another specific embodiment, at least 10%, 20%, 30%, 40%, 50%, 60%, 70%, 80%, 90%, 95%, 98% or 99% of the cells in said population are amnion derived cells that are OCT-4⁻, as determinable by RT-PCR, and VEGFR2/KDR⁺, CD9⁺, CD54⁺, CD105⁺, CD200⁺, and VE-cadherin⁻, as determinable by immunolocalization, e.g., flow cytometry. In another specific embodiment, said cells that are OCT-4⁻, as determinable by RT-PCR, and VEGFR2/KDR⁺, CD9⁺, CD54⁺, CD105⁺, CD200⁺, or VE-cadherin⁻, as determinable by immunolocalization, e.g., flow cytometry, do not express CD34, as determinable by immunolocalization, e.g., flow cytometry, after exposure to 1 to 100 ng/mL VEGF for 4 to 21 days. In another specific embodiment, said cells are also VE-cadherin⁻.

In a specific embodiment, said amnion derived cells that are OCT-4⁻, as determinable by RT-PCR, and VEGFR2/KDR⁺, CD9⁺, CD54⁺, CD105⁺, CD200⁺, or VE-cadherin⁻, as determinable by immunolocalization, e.g., flow cytometry, form sprouts or tube-like structures when said population of cells is cultured in the presence of vascular endothelial growth factor (VEGF).

The amnion derived adherent cells described herein display the above characteristics, e.g., combinations of cell surface markers and/or gene expression profiles, in primary culture, or during proliferation in medium suitable for the culture of stem cells. Such medium includes, for example, medium comprising 1 to 100% DMEM-LG (Gibco), 1 to 100% MCDB-201 (Sigma), 1 to 10% fetal calf serum (FCS) (Hyclone Laboratories), 0.1 to 5× insulin-transferrin-selenium (ITS, Sigma), 0.1 to 5× linolenic-acid-bovine-serum-albumin (LA-BSA, Sigma), 10⁻⁵ to 10⁻¹⁵ M dexamethasone (Sigma), 10⁻² to 10⁻¹⁰ M ascorbic acid 2-phosphate (Sigma), 1 to 50 ng/mL epidermal growth factor (EGF), (R&D Systems), 1 to 50 ng/mL platelet derived-growth factor (PDGF-BB) (R&D Systems), and 100 U penicillin/1000 U streptomycin. In a specific embodiment, the medium comprises 60% DMEM-LG (Gibco), 40% MCDB-201(Sigma), 2% fetal calf serum (FCS) (Hyclone Laboratories), 1× insulin-transferrin-selenium (ITS), 1× linolenic-acid-bovine-serum-albumin (LA-BSA), 10⁻⁹ M dexamethasone (Sigma), 10⁻⁴ M ascorbic acid 2-phosphate (Sigma), epidermal growth factor (EGF)10 ng/ml (R&D Systems), platelet derived-growth factor (PDGF-BB) 10 ng/ml (R&D Systems), and 100 U penicillin/1000 U streptomycin Other suitable media are described below.

The isolated populations of amnion derived adherent cells provided herein can comprise about, at least about, or no more than about, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more amnion derived adherent cells, e.g., in a container. In various embodiments, at least 10%, 20%, 30%, 40%, 50%, 60%, 70%, 80%, 90%, 95%, or 99% of the cells in the isolated cell populations provided herein are amnion derived adherent cells. That is, a population of isolated amnion derived adherent cells can comprise, e.g., as much as 1%, 5%, 10%, 20%, 30%, 40%, 50%, 60%, 70%, 80%, 90% non-AMDAC cells. In other specific embodiments, at least 25%, 35%, 45%, 50%, 60%, 75%, 85% or more of the cells in the isolated population of cells comprising amnion derived adherent cells are not OCT-4⁺.

The amnion derived adherent cells provided herein can be cultured on a substrate. In various embodiments, the substrate can be any surface on which culture and/or selection of amnion derived adherent cells, can be accomplished. Typically, the substrate is plastic, e.g., tissue culture dish or multiwell plate plastic. Tissue culture plastic can be treated, coated or imprinted with a biomolecule or synthetic mimetic agent, e.g., CELLSTART™, MESENCULT™ ACF-substrate, ornithine, or polylysine, or an extracellular matrix protein, e.g., collagen, laminin, fibronectin, vitronectin, or the like.

The amnion derived adherent cells provided herein, and populations of such cells, can be isolated from one or more placentas. Isolated amnion derived cells can be cultured and expanded to produce populations of such cells. Populations of cells comprising amnion derived adherent cells can also be cultured and expanded to produce populations of amnion derived adherent cells.

In certain embodiments, AMDACs displaying any of the above marker and/or gene expression characteristics have been passaged at least 1, 2, 3, 4, 5, 6, 7, 8, 9, 10, 11, 12, 13, 14, 15, 16, 17, 18, 19 or 20 times, or more. In certain other embodiments, AMDACs displaying any of the above marker and/or gene expression characteristics have been doubled in culture at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or at least 50 times, or more.

In a specific embodiment, the AMDACs described herein are negative for telomerase, as measured by RT-PCR and/or TRAP assays. In another specific embodiment, the AMDACs described herein do not express mRNA for telomerase reverse transcriptase (TERT) as determinable by RT-PCR. e.g., for 30 cycles. In another specific embodiment, the AMDACs described herein are NANOG⁻, as measured by RT-PCR. In another specific embodiment, the AMDACs described herein do not express mRNA for NANOG as determinable by RT-PCR, e.g., for 30 cycles. In a specific embodiment, the AMDACs described herein are negative for (sex determining region Y)-box 2 (SOX2). In another specific embodiment, the AMDACs described herein do not express mRNA for SOX2 as determinable by RT-PCR, e.g., for 30 cycles. In another specific embodiment, the AMDACs described herein are not osteogenic as measured by an osteogenic phenotype assay (see, e.g., Section 6.3.1, below). In another specific embodiment, the AMDACs described herein are not chondrogenic as measured by a chondrogenic potential assay (see, e.g., Section 6.3.2, below). In another specific embodiment, the AMDACs described herein are not osteogenic as measured by an osteogenic phenotype assay (see, e.g., Section 6.3.1, below) and are not chondrogenic as measured by a chondrogenic potential assay (see, e.g., Section 6.3.1, below).

AMDACs can exhibit one or more of the characteristics described herein as determined by RT-PCR, as demonstrated in Table 3. For example, AMDACs can exhibit one or more of such characteristics when isolated and cultured as described in Section 5.6, below.

TABLE 3

| AMDAC Marker | Positive | Negative |
|---|---|---|
| ACTA2 | X | |
| ACTC1 | X | |
| ADAMTS1 | X | |
| AMOT | X | |
| ANG | X | |
| ANGPT1 | X | |
| ANGPT2 | X | |
| ANGPT4 | | X |
| ANGPTL1 | X | |
| ANGPTL2 | X | |
| ANGPTL3 | | X |
| ANGPTL4 | X | |
| BAI1 | X | |
| BGLAP | | X |
| c-myc | X | |
| CD31 | | X |
| CD34 | | X |
| CD44 | X | |
| CD140a | X | |
| CD140b | X | |
| CD200 | X | |
| CD202b | X | |
| CD304 | X | |
| CD309 (VEGFR2/KDR) | X | |
| CDH5 | | X |
| CEACAM1 | X | |
| CHGA | X | |
| COL15A1 | X | |
| COL18A1 | X | |
| COL4A1 | X | |
| COL4A2 | X | |
| COL4A3 | X | |
| Connexin-43 | X | |
| CSF3 | X | |
| CTGF | X | |
| CXCL10 | | X |
| CXCL12 | X | |
| CXCL2 | X | |
| DLX5 | | X |
| DNMT3B | X | |
| ECGF1 | X | |
| EDG1 | X | |
| EDIL3 | X | |
| ENPP2 | X | |
| EPHB2 | X | |
| F2 | X | |
| FBLN5 | X | |
| FGA | | X |
| FGF1 | X | |
| FGF2 | X | |
| FGF4 | | X |
| FIGF | X | |
| FLT3 | | X |
| FLT4 | X | |
| FN1 | X | |
| FOXC2 | X | |
| Follistatin | X | |
| Galectin-1 | X | |
| GRN | X | |
| HEY1 | X | |
| HGF | X | |
| HLA-G | | X |
| HSPG2 | X | |
| IFNB1 | X | |
| IFNG | | X |
| IL-8 | X | |
| IL-12A | X | |
| ITGA4 | X | |
| ITGAV | X | |
| ITGB3 | X | |
| KLF-4 | X | |
| LECT1 | | X |
| LEP | | X |
| MDK | X | |
| MMP-13 | | X |
| MMP-2 | X | |
| MYOZ2 | X | |
| NANOG | | X |
| NESTIN | | X |
| NRP2 | X | |
| PDGFB | X | |
| PF4 | X | |
| PGK1 | X | |
| PLG | | X |
| POU5F1 (OCT-4) | | X |
| PRL | | X |
| PROK1 | | X |
| PROX1 | X | |
| PTN | X | |
| SEMA3F | X | |
| SERPINB5 | X | |
| SERPINC1 | X | |
| SERPINF1 | X | |
| SOX2 | | X |
| TERT | | X |
| TGFA | X | |
| TGFB1 | X | |
| THBS1 | X | |
| THBS2 | X | |
| TIE1 | X | |
| TIMP2 | X | |
| TIMP3 | X | |
| TNF | X | |
| TNFSF15 | X | |
| TNMD | | X |
| TNNC1 | X | |
| TNNT2 | X | |
| VASH1 | X | |
| VEGF | X | |
| VEGFB | X | |
| VEGFC | X | |
| VEGFR1/FLT-1 | X | |
| XLKD1 | | X |

AMDACs can exhibit one or more of the characteristics described herein as determinable by immunolocalization, e.g., flow cytometry, as demonstrated in Table 4. For example, AMDACs can exhibit one or more of such characteristics when isolated and cultured as described in Section 5.6, below.

TABLE 4

| AMDAC Marker | Positive | Negative |
|---|---|---|
| CD6 | | X |
| CD9 | X | |
| CD10 | X | |
| CD31 | | X |
| CD34 | | X |
| CD44 | X | |
| CD45 | | X |
| CD49b | X | |
| CD49c | X | |
| CD49d | X | |
| CD54 | X | |
| CD68 | X | |
| CD90 | X | |
| CD98 | X | |
| CD105 | X | |
| CD117 | | X |
| CD133 | | X |
| CD143 | | X |
| CD144 (VE-cadherin) | | X |
| CD146 | | X |
| CD166 | X | |
| CD184 | | X |
| CD200 | X | |
| CD202b | X | |
| CD271 | | X |
| CD304 | X | |
| CD309 (VEGFR2/KDR) | X | |
| CD318 | X | |
| CD349 | X | |
| CytoK | X | |
| HLA-ABC+ B2 Micro+ | X | |
| Invariant Chain+ HLA-DR- | | X |
| DP-DQ+ | | |
| PDL-1 | X | |
| VEGFR1/FLT-1 | X | |

AMDACs can exhibit one or more of the characteristics described herein as determinable by immunolocalization, e.g., immunofluorescence and/or immunohistochemistry, as demonstrated in Table 5. For example, AMDACs can exhibit one or more of such characteristics when isolated and cultured as described in Section 5.6, below.

TABLE 5

| AMDAC Marker | Positive | Negative |
|---|---|---|
| CD31 | | X |
| CD34 | | X |
| VEGFR2/KDR | X | |
| Connexin-43 | X | |
| Galectin-1 | X | |
| TEM-7 | X | |

AMDACs can exhibit one or more of the characteristics described herein as determinable by immunolocalization, e.g., membrane proteomics, as demonstrated in Table 6. For example, AMDACs can exhibit one or more of such characteristics when isolated and cultured as described in Section 5.6, below.

TABLE 6

| AMDAC Marker | Positive | Negative |
|---|---|---|
| Activin receptor type IIB | X | |
| ADAM 17 | X | |
| Alpha-actinin 1 | X | |
| Angiotensinogen | X | |
| Filamin A | X | |
| Macrophage acetylated LDL | X | |
| receptor I and II | | |
| Megalin | X | |
| Myosin heavy chain non muscle | X | |
| type A | | |
| Myosin-binding protein C | X | |
| cardiac type | | |
| Wnt-9 | X | |

AMDACs can exhibit one or more of the characteristics described herein as determinable by secretome analysis, e.g., ELISA, as demonstrated in Table 7. For example, AMDACs can exhibit one or more of such characteristics when isolated and cultured as described in Section 5.6, below.

TABLE 7

| AMDAC Marker | Positive | Negative |
|---|---|---|
| ANG | X | |
| EGF | X | |
| ENA-78 | X | |
| FGF2 | X | |
| Follistatin | X | |
| G-CSF | X | |
| GRO | X | |
| HGF | X | |
| IL-6 | X | |
| IL-8 | X | |
| Leptin | X | |
| MCP-1 | X | |
| MCP-3 | X | |
| PDGFB | X | |
| PLGF | X | |
| Rantes | X | |
| TGFB1 | X | |
| Thrombopoietin | X | |
| TIMP1 | X | |
| TIMP2 | X | |
| uPAR | X | |
| VEGF | X | |
| VEGFD | X | |

### 5.6 Populations of Amnion Derived Adherent Cells Comprising Other Cell Types

The isolated cell populations comprising amnion derived adherent cells described herein can comprise a second type of cell, e.g., placental cells that are not amnion derived adherent cells, or, e.g., cells that are not placental cells. For example, an isolated population of amnion derived adherent cells can comprise, e.g., can be combined with, a population of a second type of cells, wherein said second type of cell are, e.g., embryonic stem cells, blood cells (e.g., placental blood, placental blood cells, umbilical cord blood, umbilical cord blood cells, peripheral blood, peripheral blood cells, nucleated cells from placental blood, umbilical cord blood, or peripheral blood, and the like), stem cells isolated from blood (e.g., stem cells isolated from placental blood, umbilical cord blood or peripheral blood), placental stem cells (e.g., the placental stem cells described in U.S. Pat. No. 7,468,276, and in U.S. Patent Application Publication No. 2007/0275362, the disclosures of which are incorporated herein by reference in their entireties), nucleated cells from placental perfusate, e.g., total nucleated cells from placental perfusate, the cells described and claimed in U.S. Pat. No. 7,638,141, the disclosure of which is hereby incorporated by reference in its entirety, umbilical cord stem cells, populations of blood-derived nucleated cells, bone marrow-derived mesenchymal stromal cells, bone marrow-derived mesenchymal stem cells, bone marrow-derived hematopoietic stem cells, crude bone marrow, adult (somatic) stem cells, populations of stem cells contained within tissue, cultured cells, e.g., cultured stem cells, populations of fully-differentiated cells (e.g., chondrocytes, fibroblasts, amniotic cells, osteoblasts, muscle cells, cardiac cells, etc.), pericytes, and the like. In a specific embodiment, an isolated population of cells comprising amnion derived adherent cells comprises placental stem cells or stem cells from umbilical cord. In certain embodiments in which the second type of cell is blood or blood cells, erythrocytes have been removed from the population of cells.

In a specific embodiment, the second type of cell is a hematopoietic stem cell. Such hematopoietic stem cells can be, for example, contained within unprocessed placental blood, umbilical cord blood or peripheral blood; in total nucleated cells from placental blood, umbilical cord blood or peripheral blood; in an isolated population of CD34⁺ cells from placental blood, umbilical cord blood or peripheral blood; in unprocessed bone marrow; in total nucleated cells from bone marrow; in an isolated population of CD34⁺ cells from bone marrow, or the like.

In a another embodiment, the second cell type is a non-embryonic cell type manipulated in culture in order to express markers of pluripotency and functions associated with embryonic stem cells

In specific embodiments of the above isolated populations of amnion derived adherent cells, either or both of the amnion derived adherent cells and cells of a second type are autologous, or are allogeneic, to an intended recipient of the cells.

Further provided herein is a composition comprising amnion derived adherent cells, and a plurality of stem cells other than the amnion derived adherent cells. In a specific embodiment, the composition comprises a stem cell that is obtained from a placenta, i.e., a placental stem cell, e.g., placental stem cells as described in U.S. Pat. Nos. 7,045,148; 7,255,879; and 7,311,905, and in U.S. Patent Application Publication No. 2007/0275362, the disclosures of each of which are incorporated herein by reference in their entireties. In a specific embodiment, the placental stem cells are CD34⁻, CD10⁺ and CD105⁺. In a more specific embodiment, the placental stem cells are CD34⁻, CD10⁺, CD105⁺ and CD200⁺. In a more specific embodiment, the placental stem cells are CD34⁻, CD45⁻, CD10⁺, CD90⁺, CD105⁺ and CD200⁺. In a more specific embodiment, the placental stem cells are CD34⁻, CD45⁻, CD80⁻, CD86⁻, CD10⁺, CD90⁺, CD105⁺ and CD200⁺. In other specific embodiments, said placental stem cells are CD200⁺ and HLA-G⁺; CD73⁺, CD105⁺, and CD200⁺; CD200⁺ and OCT-4⁺; CD73⁺, CD105⁺ and HLA-G⁺; CD73⁺ and CD105⁺ and facilitate the formation of one or more embryoid-like bodies in a population of placental cells comprising said stem cell when said population is cultured under conditions that allow the formation of an embryoid-like body; or OCT-4⁺ and facilitate the formation of one or more embryoid-like bodies in a population of placental cells comprising the stem cell when said population is cultured under conditions that allow formation of embryoid-like bodies; or any combination thereof. In a more specific embodiment, said CD200⁺, HLA-G⁺ stem cells are CD34⁻, CD38⁻, CD45⁻, CD73⁺ and CD105⁺. In another more specific embodiment, said CD73⁺, CD105⁺, and CD200⁺ stem cells are CD34⁻, CD38⁻, CD45⁻, and HLA-G⁺. In another more specific embodiment, said CD200⁺, OCT-4⁺ stem cells are CD34⁻, CD38⁻, CD45⁻, CD73⁺, CD105⁺ and HLA-G⁺. In another more specific embodiment, said CD73⁺, CD105⁺ and HLA-G⁺ stem cells are CD34⁻, CD45⁻, OCT-4⁺ and CD200⁺. In another more specific embodiment, said CD73⁺ and CD105⁺ stem cells are OCT-4⁺, CD34⁻, CD38⁻ and CD45⁻. In another more specific embodiment, said OCT-4⁺ stem cells are CD73⁺, CD105⁺, CD200⁺, CD34⁻, CD38⁻, and CD45⁻. In another more specific embodiment, the placental stem cells are maternal in origin (that is, have the maternal genotype). In another more specific embodiment, the placental stem cells are fetal in origin (that is, have the fetal genotype).

In another specific embodiment, the composition comprises amnion derived adherent cells, and embryonic stem cells. In another specific embodiment, the composition comprises amnion derived adherent cells and mesenchymal stromal or stem cells, e.g., bone marrow-derived mesenchymal stromal or stem cells. In another specific embodiment, the composition comprises bone marrow-derived hematopoietic stem cells. In another specific embodiment, the composition comprises amnion derived adherent cells and hematopoietic progenitor cells, e.g., hematopoietic progenitor cells from bone marrow, fetal blood, umbilical cord blood, placental blood, and/or peripheral blood. In another specific embodiment, the composition comprises amnion derived adherent cells and somatic stem cells. In a more specific embodiment, said somatic stem cell is a neural stem cell, a hepatic stem cell, a pancreatic stem cell, an endothelial stem cell, a cardiac stem cell, or a muscle stem cell.

In other specific embodiments, the second type of cells comprise about, at least, or no more than, 10%, 15%, 20%, 25%, 30%, 35%, 40%, 45%, or 50% of cells in said population. In other specific embodiments, the AMDACs in said composition comprise at least 50%, 55%, 60%, 65%, 70%, 75%, 80%, 85% or 90% of cells in said composition. In other specific embodiments, the amnion derived adherent cells comprise about, at least, or no more than, 10%, 15%, 20%, 25%, 30%, 35%, 40%, or 45% of cells in said population. In other specific embodiments, at least 25%, 35%, 45%, 50%, 60%, 75%, 85% or more of the cells in said population are not OCT-4⁺.

Cells in an isolated population of amnion derived adherent cells can be combined with a plurality of cells of another type, e.g., with a population of stem cells, in a ratio of about 100,000,000:1, 50,000,000:1, 20,000,000:1, 10,000,000:1, 5,000,000:1, 2,000,000:1, 1,000.000:1, 500,000:1, 200,000:1, 100,000:1, 50,000:1, 20,000:1, 10,000:1, 5,000:1, 2,000:1, 1,000:1, 500:1, 200:1, 100:1, 50:1, 20:1, 10:1, 5:1.2:1, 1:1; 1:2; 1:5; 1:10; 1:100; 1:200; 1:500; 1:1,000; 1:2,000; 1:5,000; 1:10,000; 1:20,000; 1:50,000; 1:100,000; 1:500,000; 1:1,000,000; 1:2,000,000; 1:5,000,000; 1:10,000,000; 1:20,000,000; 1:50,000,000; or about 1:100,000,000, comparing numbers of total nucleated cells in each population. Cells in an isolated population of amnion derived adherent cells can be combined with a plurality of cells of a plurality of cell types, as well.

### 5.7 Growth in Culture

The growth of the amnion derived adherent cells described herein, as for any mammalian cell, depends in part upon the particular medium selected for growth. Under optimum conditions, amnion derived adherent cells typically double in number in approximately 24 hours. During culture, the amnion derived adherent cells described herein adhere to a substrate in culture, e.g. the surface of a tissue culture container (e.g., tissue culture dish plastic, fibronectin-coated plastic, and the like) and form a monolayer. Typically, the cells establish in culture within 2-7 days after digestion of the amnion. They proliferate at approximately 0.4 to 1.2 population doublings per day and can undergo at least 30 to 50 population doublings. The cells display a mesenchymal/fibroblastic cell-like phenotype during subconfluence and expansion, and a cuboidal/cobblestone-like appearance at confluence, and proliferation in culture is strongly contact-inhibited. Populations of amnion-derived adherent cells can form embryoid bodies during expansion in culture.

### 5.8 Methods of Obtaining Amnion-Derived Adherent Cells

The amnion derived adherent cells, and populations of cells comprising the amnion derived adherent cells, can be produced, e.g., isolated from other cells or cell populations, for example, through particular methods of digestion of amnion tissue, optionally followed by assessment of the resulting cells or cell population for the presence or absence of markers, or combinations of markers, characteristics of amnion derived adherent cells, or by obtaining amnion cells and selecting on the basis of markers characteristic of amnion derived adherent cells.

The amnion derived adherent cells, and isolated populations of cells comprising the amnion derived adherent cells, provided herein can be produced by, e.g., digestion of amnion tissue followed by selection for adherent cells. In one embodiment, for instance, isolated amnion derived adherent cells, or an isolated population of cells comprising amnion derived adherent cells, can be produced by (1) digesting amnion tissue with a first enzyme to dissociate cells from the epithelial layer of the amnion from cells from the mesenchymal layer of the amnion; (2) subsequently digesting the mesenchymal layer of the amnion with a second enzyme to form a single-cell suspension; (3) culturing cells in said single-cell suspension on a tissue culture surface, e.g., tissue culture plastic; and (4) selecting cells that adhere to said surface after a change of medium, thereby producing an isolated population of cells comprising amnion derived adherent cells. In a specific embodiment, said first enzyme is trypsin. In a more specific embodiment, said trypsin is used at a concentration of 0.25% trypsin (w/v), in 5-20, e.g., 10 milliliters solution per gram of amnion tissue to be digested. In another more specific embodiment, said digesting with trypsin is allowed to proceed for about 15 minutes at 37° C. and is repeated up to three times. In another specific embodiment, said second enzyme is collagenase. In a more specific embodiment, said collagenase is used at a concentration between 50 and 500 U/L in 5 mL per gram of amnion tissue to be digested. In another more specific embodiment, said digesting with collagenase is allowed to proceed for about 45-60 minutes at 37° C. In another specific embodiment, the single-cell suspension formed after digestion with collagenase is filtered through, e.g., a 75 μM-150 μM filter between step (2) and step (3). In another specific embodiment, said first enzyme is trypsin, and said second enzyme is collagenase.

An isolated population of cells comprising amnion derived adherent cells can, in another embodiment, be obtained by selecting cells from amnion, e.g., cells obtained by digesting amnion tissue as described elsewhere herein, that display one or more characteristics of an amnion derived adherent cell. In one embodiment, for example, a cell population is produced by a method comprising selecting identifying cells that are (a) negative for OCT-4, as determinable by RT-PCR, and (b) positive for one or more of VEGFR2/KDR, CD9, CD54, CD105, CD200, as determinable or selectable by immunolocalization, e.g., flow cytometry; and isolating said cells from other cells to form a cell population. In a specific embodiment, said amnion cells are additionally VE-cadherin⁻. In a specific embodiment, a cell population is produced by selecting amnion cells that are (a) negative for OCT-4, as determinable by RT-PCR, and VE-cadherin, as determinable by immunolocalization, e.g., flow cytometry, and (b) positive for each of VEGFR2/KDR, CD9, CD54, CD105, CD200, as determinable by immunolocalization, e.g., flow cytometry; and isolating said cells from other cells to form a cell population. In certain embodiments, selection by immunolocalization, e.g., flow cytometry, is performed before selection by RT-PCR. In another specific embodiment, said selecting comprises selecting cells that do not express cellular marker CD34 after culture for 4 to 21 days in the presence of 1 to 100 ng/mL VEGF.

In another embodiment, for example, a cell population is produced by a method comprising selecting amnion cells that are adherent to tissue culture plastic and are OCT-4, as determinable by RT-PCR, and VEGFR1/Flt-1⁺ and VEGFR2/KDR⁺, as determinable by immunolocalization, e.g., flow cytometry, and isolating said cells from other cells to form a cell population. In a specific embodiment, a cell population is produced by a method comprising selecting amnion cells that are OCT-4⁻, as determinable by RT-PCR, and VEGFR1/Flt-1⁺, VEGFR2/KDR⁺, and HLA-G⁻, as determinable by immunolocalization, e.g., flow cytometry. In another specific embodiment, said cell population is produced by selecting amnion cells that are additionally one or more, or all, of CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and/or CXCR4⁻ (chemokine (C-X-C motif) receptor 4) as determinable by immunolocalization, e.g., flow cytometry, and isolating the cells from cells that do not display one or more of these characteristics. In another specific embodiment, said cell population is produced by selecting amnion cells that are additionally VE-cadherin⁻ as determinable by immunolocalization, e.g., flow cytometry, and isolating the cells from cells that are VE-cadherin⁺. In another specific embodiment, said cell population is produced by selecting amnion cells that are additionally CD105⁺ and CD200⁺ as determinable by immunolocalization, e.g., flow cytometry, and isolating the cells from cells that are CD105⁻ or CD200⁻. In another specific embodiment, said cell does not express CD34 as detected by immunolocalization, e.g., flow cytometry, after exposure to 1 to 100 ng/mL VEGF for 4 to 21 days.

In the selection of cells, it is not necessary to test an entire population of cells for characteristics specific to amnion derived adherent cells. Instead, one or more aliquots of cells (e.g., about 0.5%-2%) of a population of cells may be tested for such characteristics, and the results can be attributed to the remaining cells in the population.

Selected cells can be confirmed to be the amnion derived adherent cells provided herein by culturing a sample of the cells (e.g., about 10⁴ to about 10⁵ cells) on a substrate, e.g., MATRIGELT™, for 4 to 14, e.g., 7, days in the presence of VEGF (e.g., about 50 ng/mL), and visually inspecting the cells for the appearance of sprouts and/or cellular networks.

Amnion derived adherent cells can be selected by the above markers using any method known in the art of cell selection. For example, the adherent cells can be selected using an antibody or antibodies to one or more cell surface markers, for example, in immunolocalization, e.g., flow cytometry or FACS. Selection can be accomplished using antibodies in conjunction with magnetic beads. Antibodies that are specific for certain markers are known in the art and are available commercially, e.g., antibodies to CD9 (Abcam); CD54 (Abcam); CD105 (Abcam; BioDesign International, Saco, Me., etc.); CD200 (Abcam) cytokeratin (SigmaAldrich). Antibodies to other markers are also available commercially, e.g., CD34, CD38 and CD45 are available from, e.g., StemCell Technologies or BioDesign International. Primers to OCT-4 sequences suitable for RT-PCR can be obtained commercially, e.g., from Millipore or Invitrogen, or can be readily derived from the human sequence in GenBank Accession No. DQ486513.

Detailed methods of obtaining placenta and amnion tissue from placenta, and treating such tissue in order to obtain amnion derived adherent cells, are provided below.

5.8.1 Cell Collection Composition

Generally, cells can be obtained from amnion from a mammalian placenta, e.g., a human placenta, using a physiologically-acceptable solution, e.g., a cell collection composition. In certain embodiments, the cell collection composition prevents or suppresses apoptosis, prevents or suppresses cell death, lysis, decomposition and the like. A cell collection composition is described in detail in related U.S. Patent Application Publication No. 2007/0190042, entitled “Improved Medium for Collecting Placental Stem Cells and Preserving Organs,” the disclosure of which is incorporated herein by reference in its entirety.

The cell collection composition can comprise any physiologically-acceptable solution suitable for the collection and/or culture of amnion derived adherent cells, for example, a saline solution (e.g., phosphate-buffered saline, Kreb's solution, modified Kreb's solution, Eagle's solution, 0.9% NaCl. etc.), a culture medium (e.g., DMEM, H.DMEM, etc.), and the like, with or without the addition of a buffering component, e.g., 4-(2-hydroxyethyl)-1-piperazineethanesulfonic acid (HEPES).

The cell collection composition can comprise one or more components that tend to preserve cells, e.g., amnion derived adherent cells, that is, prevent the cells from dying, or delay the death of the cells, reduce the number of cells in a population of cells that die, or the like, from the time of collection to the time of culturing. Such components can be, e.g., an apoptosis inhibitor (e.g., a caspase inhibitor or JNK inhibitor); a vasodilator (e.g., magnesium sulfate, an antihypertensive drug, atrial natriuretic peptide (ANP), adrenocorticotropin, corticotropin-releasing hormone, sodium nitroprusside, hydralazine, adenosine triphosphate, adenosine, indomethacin or magnesium sulfate, a phosphodiesterase inhibitor, etc.); a necrosis inhibitor (e.g., 2-(1H-Indol-3-yl)-3-pentyl amino-maleimide, pyrrolidine dithiocarbamate, or clonazepam); a TNF-α inhibitor; and/or an oxygen-carrying perfluorocarbon (e.g., pertluorooctyl bromide, perfluorodecyl bromide, etc.).

The cell collection composition can comprise one or more tissue-degrading enzymes, e.g. a metalloprotease, a serine protease, a neutral protease, an RNase, or a DNase, or the like. Such enzymes include, but are not limited to, collagenases (e.g., collagenase I, II, III or IV, a collagenase from Clostridium histolyticum, etc.); dispase, thermolysin, elastase, trypsin, LIBERASE™, hyaluronidase, and the like. The use of cell collection compositions comprising tissue-digesting enzymes is discussed in more detail below.

The cell collection composition can comprise a bacteriocidally or bacteriostatically effective amount of an antibiotic. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram(+) and/or Gram(−) bacteria, e.g., Pseudomonas aeruginosa, Staphylococcus aureus, and the like.

The cell collection composition can also comprise one or more of the following compounds: adenosine (about 1 mM to about 50 mM); D-glucose (about 20 mM to about 100 mM); magnesium ions (about 1 mM to about 50 mM); a macromolecule of molecular weight greater than 20,000 daltons, in one embodiment, present in an amount sufficient to maintain endothelial integrity and cellular viability (e.g., a synthetic or naturally occurring colloid, a polysaccharide such as dextran or a polyethylene glycol present at about 25 g/l to about 100 g/l, or about 40 g/l to about 60 g/l); an antioxidant (e.g., butylated hydroxyanisole, butylated hydroxytoluene, glutathione, vitamin C or vitamin E present at about 25 μM to about 100 μM); a reducing agent (e.g., N-acetylcysteine present at about 0.1 mM to about 5 mM); an agent that prevents calcium entry into cells (e.g., verapamil present at about 2 μM to about 25 μM); nitroglycerin (e.g., about 0.05 g/L to about 0.2 g/L); an anticoagulant, in one embodiment, present in an amount sufficient to help prevent clotting of residual blood (e.g., heparin or hirudin present at a concentration of about 1000 units/l to about 100,000 units/l); or an amiloride containing compound (e.g., amiloride, ethyl isopropyl amiloride, hexamethylene amiloride, dimethyl amiloride or isobutyl amiloride present at about 1.0 μM to about 5 μM).

The amnion derived adherent cells described herein can also be collected, e.g., during and after digestion as described below, into a simple physiologically-acceptable buffer, e.g., phosphate-buffered saline, a 0.9% NaCl solution, cell culture medium, or the like.

5.8.2 Collection and Handling of Placenta

Generally, a human placenta is recovered shortly after its expulsion after birth, or after, e.g., Caesarian section. In a preferred embodiment, the placenta is recovered from a patient after informed consent and after a complete medical history of the patient is obtained and is associated with the placenta. Preferably, the medical history continues after delivery. Such a medical history can be used to coordinate subsequent use of the placenta or cells harvested therefrom. For example, human amnion derived adherent cells can be used, in light of the medical history, for personalized medicine for the infant, or a close relative, associated with the placenta, or for parents, siblings, or other relatives of the infant.

Prior to recovery of amnion derived adherent cells, the umbilical cord blood and placental blood are removed. In certain embodiments, after delivery, the cord blood in the placenta is recovered. The placenta can be subjected to a conventional cord blood recovery process. Typically a needle or cannula is used, with the aid of gravity, to exsanguinate the placenta (see, e.g., Anderson, U.S. Pat. No. 5,372,581; Hessel et al., U.S. Pat. No. 5,415,665). The needle or cannula is usually placed in the umbilical vein and the placenta can be gently massaged to aid in draining cord blood from the placenta. Such cord blood recovery may be performed commercially, e.g., LifeBank USA, Cedar Knolls, N.J., ViaCord, Cord Blood Registry and Cryocell. Preferably, the placenta is gravity drained without further manipulation so as to minimize tissue disruption during cord blood recovery.

Typically, a placenta is transported from the delivery or birthing room to another location, e.g., a laboratory, for recovery of cord blood and collection of cells by, e.g., tissue dissociation. The placenta is preferably transported in a sterile, thermally insulated transport device (maintaining the temperature of the placenta between 20-28° C.), for example, by placing the placenta, with clamped proximal umbilical cord, in a sterile zip-lock plastic bag, which is then placed in an insulated container. In another embodiment, the placenta is transported in a cord blood collection kit substantially as described in U.S. Pat. No. 7,147,626. Preferably, the placenta is delivered to the laboratory four to twenty-four hours following delivery. In certain embodiments, the proximal umbilical cord is clamped, preferably within 4-5 cm (centimeter) of the insertion into the placental disc prior to cord blood recovery. In other embodiments, the proximal umbilical cord is clamped after cord blood recovery but prior to further processing of the placenta.

The placenta, prior to cell collection, can be stored under sterile conditions and at a temperature of. e.g., 4 to 25° C. (centigrade), e.g., at room temperature. The placenta may be stored for, e.g. a period of for zero to twenty-four hours, up to forty-eight hours, or longer than forty eight hours, prior to perfusing the placenta to remove any residual cord blood. In one embodiment, the placenta is harvested from between about zero hours to about two hours post-expulsion. The placenta can be stored in an anticoagulant solution at a temperature of, e.g., 4 to 25° C. (centigrade). Suitable anticoagulant solutions are well known in the art. For example, a solution of sodium citrate, heparin or warfarin sodium can be used. In a preferred embodiment, the anticoagulant solution comprises a solution of heparin (e.g., 1% w/w in 1:1000 solution). The exsanguinated placenta is preferably stored for no more than 36 hours before cells are collected.

See, e.g., U.S. Pat. No. 7,638,141, the disclosure of which is hereby incorporated by reference in its entirety, for additional information regarding collection and handling of placenta.

5.8.3 Physical Disruption and Enzymatic Digestion of Amnion Tissue

In one embodiment, the amnion is separated from the rest of the placenta, e.g., by blunt dissection, e.g., using the fingers. The amnion can be dissected, e.g., into parts or tissue segments, prior to enzymatic digestion and adherent cell recovery. Amnion derived adherent cells can be obtained from a whole amnion, or from a small segment of amnion, e.g., a segment of amnion that is about 1, 2, 3, 4, 5, 6, 7, 8, 9, 10, 20, 30, 40, 50, 60, 70, 80, 90, 100, 200, 300, 400, 500, 600, 700, 800, 900 or about 1000 square millimeters in area.

Amnion derived adherent cells can generally be collected from a placental amnion or a portion thereof, at any time within about the first three days post-expulsion, but preferably between about 0 hours and 48 hours after expulsion, or about 8 hours and about 18 hours post-expulsion.

AMDACs can, for example, be isolated using a specific two-step isolation method comprising digestion with trypsin followed by digestion with collagenase. For example, provided herein is a method of isolating amnion derived adherent cells comprising digesting an amniotic membrane or portion thereof with trypsin such that epithelial cells are released from said amniotic membrane; removing the amniotic membrane or portion thereof from said epithelial cells; further digesting the amniotic membrane or portion thereof with collagenase. In a specific embodiment, digestion of the amniotic membrane or portion thereof with trypsin is repeated at least once. In another specific embodiment, digestion of the amniotic membrane or portion thereof with collagenase is repeated at least once. In another specific embodiment, the trypsin is at about 0.1%-1.0% (final concentration). In a more specific embodiment, the trypsin is at about 0.25% (final concentration). In another specific embodiment, the collagenase is at about 50 U/mL to about 1000 U/mL (final concentration). In a more specific embodiment, the collagenase is at about 125 U/mL (final concentration).

In one embodiment, for example, amnion derived adherent cells can be obtained as follows. The amniotic membrane is isolated from the placenta via, e.g., dissection, then cut into segments approximately 0.1″×0.1″ to about 5″×5″, e.g., 2″×2″, in size. The epithelial monolayer is removed from the fetal side of the amniotic membrane by trypsinization, e.g., triple trypsinization as follows. The segments of amniotic membrane are placed into a container with warm (e.g., about 20° C. to about 37° C.) trypsin-EDTA solution (0.25%). The volume of the trypsin solution can range from about 5 mL per gram of amniotic membrane to about 50 mL per gram of amniotic membrane. The container is agitated for about 5 minutes to about 30 minutes, e.g., 15 minutes, while maintaining the temperature constant. The segments of amniotic membrane are then separated from the trypsin solution by any appropriate method, such as manually removing the amnion segments, or by filtration. The trypsinization step can be repeated at least one more time. In one embodiment, the trypsinization step is repeated twice (for triple trypsinization) or three times (for quadruple trypsinization).

In one embodiment, upon completion of the final trypsinization, the segments of amniotic membrane are placed back into warm (e.g., about 20° C. to about 37° C.) trypsin neutralization solution (e.g., at a volume of about 5 mL per gram of amniotic membrane to about 50 mL per gram of amniotic membrane), such as phosphate-buffered saline (PBS)/10% fetal bovine serum (FBS), PBS/5% FBS or PBS/3% FBS. The container is agitated for about 5 seconds to about 30 minutes, e.g., 5, 10 or 15 minutes. The segments of amniotic membrane are then separated from the trypsin neutralization solution by any appropriate method, such as manually removing the amnion segments, or by filtration. The segments of amniotic membrane are placed into the container filled with warm (e.g., about 20° C. to about 37° C.) PBS, pH 7.2 solution (e.g., at a volume of about 5 mL per gram of amniotic membrane to about 50 mL per gram of amniotic membrane). The container is agitated for about 5 seconds to about 30 minutes, e.g., 5, 10 or 15 minutes. The amniotic membrane segments are then separated from the PBS as described above.

The segments of amniotic membrane are then placed into warm (e.g., about 20° C. to about 37° C.) digestion solution. The volume of digestion solution can range from about 5 mL per gram of amnion to about 50 mL per gram of amnion. Digestion solutions comprise digestion enzymes in an appropriate culture medium, such as DMEM. Typical digestion solutions include collagenase type I (about 50 U/mL to about 500 U/mL). Digestion solutions for this stage of the process do not generally comprise trypsin. Agitation is generally at 37° C. until amnion digestion is substantially complete, as evidenced, e.g., by complete dissolution of the amniotic membrane yielding a homogeneous suspension (approximately 10 minutes to about 90 minutes). Warm PBS/5% FBS is then added at a ratio of about 1 mL per gram of amniotic tissue to about 50 mL per gram of amniotic tissue and agitated for about 2 minutes to about 5 minutes. The cell suspension is then filtered to remove any un-digested tissue using, e.g., a 40 μm to 100 μm filter. The cells are suspended in warm PBS (about 1 mL to about 500 mL), and then centrifuged at 200×g to about 400×g for about 5 minutes to about 30 minutes, e.g. 300×g for about 15 minutes at 20° C. After centrifugation, the supernatant is removed and the cells are resuspended in a suitable culture medium. The cell suspension can be filtered (40 μm to 70 μm filter) to remove any remaining undigested tissue, yielding a single cell suspension. The remaining undigested amnion, in this embodiment, can be discarded.

In this embodiment, cells in suspension are collected and cultured as described elsewhere herein to produce isolated amnion derived adherent cells, and populations of such cells. For example, in one embodiment, the cells in suspension can be cultured and amnion derived adherent cells can be separated from non-adherent cells in said culture to produce an enriched population of amnion derived adherent cells. In more specific embodiments, at least 50%, 55%, 60%, 65%, 70%, 75%, 80%, 85%, 90%, 95%, 98% or 99% of cells in said enriched population of amnion derived adherent cells are said amnion derived adherent cells.

In any of the digestion protocols herein, the cell suspension obtained by digestion can be filtered, e.g., through a filter comprising pores from about 50 μm to about 150 μm, e.g., from about 75 μm to about 125 μm. In a more specific embodiment, the cell suspension can be filtered through two or more filters, e.g., a 125 μm filter and a 75 μm filter.

In conjunction with any of the methods described herein, AMDACs can be isolated from the cells released during digestion by selecting cells that express one or more characteristics of AMDACs, as described in Section 5.3, above.

In one embodiment, AMDACs can be isolated using, in order, a first enzyme and a second enzyme, wherein the first enzyme used in the method is not collagenase, and wherein the second enzyme used in the method is not trypsin. In another embodiment, the digestion step used to isolate AMDACs does not use a combination of any two or more of collagenase, dispase or hyaluronidase. In another embodiment, the AMDACs are not isolated via explant culturing to allow the cells to be detected by growth, replication, or migration out of the explants.

In another embodiment, deoxyribonuclease (DNase) is not used during the isolation of AMDACs. For example, in certain embodiments, DNase is not used following the collagenase digestion step of the isolation.

5.8.4 Isolation, Sorting, and Characterization of Amnion Derived Adherent Cells

Cell pellets can be resuspended in fresh cell collection composition, as described above, or a medium suitable for cell maintenance, e.g., Dulbecco's Modified Eagle's Medium (DMEM); Iscove's Modified Dulbecco's Medium (IMDM), e.g. IMDM serum-free medium containing 2 U/mL heparin and 2 mM EDTA (GibcoBRL, NY); a mixture of buffer (e.g. PBS, HBSS) with FBS (e.g. 2% v/v); or the like.

Amnion derived adherent cells that have been cultured, e.g., on a surface, e.g., on tissue culture plastic, with or without additional extracellular matrix coating such as fibronectin, can be passaged or isolated by differential adherence. For example, a cell suspension obtained as described in Section 5.6.3, above, can be cultured, e.g., for 3-7 days in culture medium on tissue culture plastic. During culture, a plurality of cells in the suspension adhere to the culture surface, and nonadherent cells are removed during medium exchange.

The number and type of cells collected from amnion can be monitored, for example, by measuring changes in morphology and cell surface markers using standard cell detection techniques such as immunolocalization, e.g., flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. For example, using one or more antibodies to CD34, one can determine, using the techniques above, whether a cell comprises a detectable amount of CD34; if so, the cell is CD34⁺.

Amnion derived adherent cells can be isolated by Ficoll separation, e.g., Ficoll gradient centrifugation. Such centrifugation can follow any standard protocol for centrifugation speed, etc. In one embodiment, for example, cells recovered after digestion of the amnion are separated using a Ficoll gradient by centrifugation at 5000×g for 15 minutes at room temperature and cell layers of interest are collected for further processing.

Amnion-derived cells, e.g., cells that have been isolated by Ficoll separation, differential adherence, or a combination of both can be sorted using a fluorescence activated cell sorter (FACS). Fluorescence activated cell sorting (FACS) is a well-known method for separating particles, including cells, based on the fluorescent properties of the particles (see, e.g., Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In one sorting scheme, cells from placenta, e.g., amnion derived adherent cells, can be sorted on the basis of expression of the markers CD49f, VEGFR2/KDR, and/or Flt-1/VEGFR1. Preferably the cells are identified as being OCT-4⁻, e.g., by determining the expression of OCT-4 by RT-PCR in a sample of the cells, wherein the cells are OCT-4⁻ if the cells in the sample fail to show detectable production of mRNA for OCT-4 after 30 cycles. For example, cells from amnion that are VEGFR2/KDR⁺ and VEGFR1/Flt-1⁺ can be sorted from cells that are one or more of VEGFR2/KDR⁻, and VEGFR1/Flt-1⁺, CD9⁺, CD54⁺, CD 105⁺, CD200⁺, and/or VE-cadherin⁻. In a specific embodiment, amnion-derived, tissue culture plastic-adherent cells that are one or more of CD49f⁺, VEGFR2/KDR⁺, CD9⁺, CD54⁺, CD105⁺, CD200⁺, and/or VE-cadherin⁻, or cells that are VEGFR2/KDR⁺, CD9⁺, CD54⁺, CD105⁺, CD200⁺, and VE-cadherin⁻, are sorted away from cells not expressing one or more of such marker(s), and selected. In another specific embodiment, CD49f⁺, VEGFR2/KDR⁺, VEGFR1/Flt-1⁺ cells that are additionally one or more, or all, of CD31⁺, CD34⁺, CD45⁺, CD133⁻, and/or Tie-2⁺ are sorted from cells that do not display one or more, or any, of such characteristics. In another specific embodiment, VEGFR2/KDR⁺, VEGFR1/Flt-1⁺ cells that are additionally one or more, or all, of CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and/or CXCR4⁻, are sorted from cells that do not display one or more, or any, of such characteristics.

Selection for amnion derived adherent cells can be performed on a cell suspension resulting from digestion, or on isolated cells collected from digestate, e.g., by centrifugation or separation using flow cytometry. Selection by expressed markers can be accomplished alone or, e.g., in connection with procedures to select cells on the basis of their adherence properties in culture. For example, an adherence selection can be accomplished before or after sorting on the basis of marker expression.

With respect to antibody-mediated detection and sorting of amnion cells, any antibody, specific for a particular marker, can be used, in combination with any fluorophore or other label suitable for the detection and sorting of cells (e.g., fluorescence-activated cell sorting). Antibody/fluorophore combinations to specific markers include, but are not limited to, fluorescein isothiocyanate (FITC) conjugated monoclonal antibodies against CD105 (available from R&D Systems Inc., Minneapolis, Minn.); phycoerythrin (PE) conjugated monoclonal antibodies against CD200 (BD Biosciences Pharmingen); VEGFR2/KDR-Biotin (CD309, Abcam), and the like. Antibodies to any of the markers disclosed herein can be labeled with any standard label for antibodies that facilitates detection of the antibodies, including, e.g., horseradish peroxidase, alkaline phosphatase, β-galactosidase, acetylcholinesterase streptavidin/biotin, avidin/biotin, umbelliferone, fluorescein, fluorescein isothiocyanate (FITC), rhodamine, dichlorotriazinylamine fluorescein, dansyl chloride or phycoerythrin (PE), luminol, luciferase, luciferin, and aequorin, and examples of suitable radioactive material include ¹²⁵I, ¹³¹I, ³⁵S or ³H.

Amnion derived adherent cells can be labeled with an antibody to a single marker and detected and/sorted based on the single marker, or can be simultaneously labeled with multiple antibodies to a plurality of different markers and sorted based on the plurality of markers.

In another embodiment, magnetic beads can be used to separate cells, e.g., to separate the amnion derived adherent cells described herein from other amnion cells. The cells may be sorted using a magnetic activated cell sorting (MACS) technique, a method for separating particles based on their ability to bind magnetic beads (0.5-100 μm diameter). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

Amnion derived adherent cells can be assessed for viability, proliferation potential, and longevity using standard techniques known in the art, such as trypan blue exclusion assay, fluorescein diacetate uptake assay, propidium iodide uptake assay (to assess viability); and thymidine uptake assay or MTT cell proliferation assay (to assess proliferation). Longevity may be determined by methods well known in the art, such as by determining the maximum number of population doublings in an extended culture.

Amnion derived adherent cells, can also be separated from other placental or amnion cells using other techniques known in the art, e.g., selective growth of desired cells (positive selection), selective destruction of unwanted cells (negative selection); separation based upon differential cell agglutinability in the mixed population as, for example, with soybean agglutinin; freeze-thaw procedures; filtration; conventional and zonal centrifugation; centrifugal elutriation (counter-streaming centrifugation); unit gravity separation; countercurrent distribution; electrophoresis; and the like.

### 5.9 Culture of Amnion Derived Adherent Cells

5.9.1 Culture Media

Isolated amnion derived adherent cells, or populations of such cells, can be used to initiate, or seed, cell cultures. Cells are generally transferred to sterile tissue culture vessels either uncoated or coated with extracellular matrix or biomolecules such as laminin, collagen (e.g., native or denatured), gelatin, fibronectin, ornithine, vitronectin, and extracellular membrane protein (e.g., MATRIGEL™ (BD Discovery Labware, Bedford, Mass.)).

AMDACs can, for example, be established in media suitable for the culture of stem cells. Establishment media can, for example, include EGM-2 medium (Lonza), DMEM+10% FBS, or medium comprising 60% DMEM-LG (Gibco), 40% MCDB-201(Sigma), 2% fetal calf serum (FCS) (Hyclone Laboratories), 1× insulin-transferrin-selenium (ITS), 1× lenoleic-acid-bovine-serum-albumin (LA-BSA), 10⁻⁹ M dexamethasone (Sigma), 10⁻⁴M ascorbic acid 2-phosphate (Sigma), epidermal growth factor (EGF) 10 ng/ml (R&D Systems), platelet derived-growth factor (PDGF-BB) 10 ng/ml (R&D Systems), and 100 U penicillin/1000 U streptomycin (referred to herein as “standard medium”).

Amnion derived adherent cells can be cultured in any medium, and under any conditions, recognized in the art as acceptable for the culture of cells, e.g., adherent placental stem cells. Preferably, the culture medium comprises serum. In various embodiments, media for the culture or subculture of AMDACs includes STEMPRO® (Invitrogen), MSCM-sf (ScienCell, Carlsbad, Calif.), MESENCULT®-ACE medium (StemCell Technologies, Vancouver. Canada), standard medium, standard medium lacking EGF, standard medium lacking PDGF, DMEM+10% FBS, EGM-2 (Lonza), EGM-2MV (Lonza), 2%, 10% and 20% ES media, ES-SSR medium, or α-MEM-20% FBS. Medium acceptable for the culture of amnion derived adherent cells includes, e.g., DMEM, IMDM, DMEM (high or low glucose), Eagle's basal medium, Ham's F10 medium (F10), Ham's F-12 medium (F12), Iscove's modified Dulbecco's medium, Mesenchymal Stem Cell Growth Medium (MSCGM Lonza), ADVANCESTEM™ Medium (Hyclone), KNOCKOUT™ DMEM (Invitrogen), Leibovitz's L-15 medium, MCDB, DMEM/F12, RPMI 1640, advanced DMEM (Gibco), DMEM/MCDB201 (Sigma), and CELL-GRO FREE, or the like. In various embodiments, for example, DMEM-LG (Dulbecco's Modified Essential Medium, low glucose)/MCDB 201 (chick fibroblast basal medium) containing ITS (insulin-transferrin-selenium), LA+BSA (linoleic acid-bovine serum albumin), dextrose, L-ascorbic acid, PDGF, EGF, IGF-1, and penicillin/streptomycin; DMEM-HG (high glucose) comprising about 2 to about 20%, e.g., about 10%, fetal bovine serum (FBS; e.g. defined fetal bovine serum, Hyclone, Logan Utah); DMEM-HG comprising about 2 to about 20%, e.g., about 15%, FBS; IMDM (Iscove's modified Dulbecco's medium) comprising about 2 to about 20%, e.g., about 10%, FBS, about 2 to about 20%, e.g., about 10%, horse serum, and hydrocortisone; M199 comprising about 2 to about 20%, e.g., about 10%, FBS, EGF, and heparin; α-MEM (minimal essential medium) comprising about 2 to about 20%, e.g., about 10%, FBS, GLUTAMAX™ and gentamicin; DMEM comprising 10% FBS, GLUTAMAX™ and gentamicin; DMEM-LG comprising about 2 to about 20%, e.g., about 15%, (v/v) fetal bovine serum (e.g., defined fetal bovine serum, Hyclone, Logan Utah), antibiotics/antimycotics (e.g., penicillin at about 100 Units/milliliter, streptomycin at 100 micrograms/milliliter, and/or amphotericin B at 0.25 micrograms/milliliter (Invitrogen, Carlsbad, Calif.)), and 0.001% (v/v) β-mercaptoethanol (Sigma, St. Louis Mo.); KNOCKOUT™-DMEM basal medium supplemented with 2 to 20% FBS, non-essential amino acid (Invitrogen), beta-mercaptoethanol, KNOCKOUT™ basal medium supplemented with KNOCKOUT™ Serum Replacement, alpha-MEM comprising 2 to 20% FBS, EBM2™ basal medium supplemented with EGF, VEGF, bFGF, R3-IGF-1, hydrocortisone, heparin, ascorbic acid, FBS, gentamicin), or the like.

The culture medium can be supplemented with one or more components including, for example, serum (e.g. FCS or FBS, e.g., about 2-20% (v/v); equine (horse) serum (ES); human serum (HS)); beta-mercaptoethanol (BME), preferably about 0.001% (v/v); one or more growth factors, for example, platelet-derived growth factor (PDGF), epidermal growth factor (FOE), basic fibroblast growth factor (bFGF), insulin-like growth factor-1 (IGF-1), leukemia inhibitory factor (LIF), vascular endothelial growth factor (VEGF), and erythropoietin (EPO); amino acids, including L-valine; and one or more antibiotic and/or antimycotic agents to control microbial contamination, such as, for example, penicillin G, streptomycin sulfate, amphotericin B, gentamicin, and nystatin, either alone or in combination.

Amnion derived adherent cells (AMDACs) can be cultured in standard tissue culture conditions, e.g., in tissue culture dishes or multiwell plates. The cells can also be cultured using a hanging drop method. In this method, the cells are suspended at about 1×10⁴ cells per mL in about 5 mL of medium, and one or more drops of the medium are placed on the inside of the lid of a tissue culture container, e.g., a 100 mL Petri dish. The drops can be, e.g., single drops, or multiple drops from, e.g., a multichannel pipetter. The lid is carefully inverted and placed on top of the bottom of the dish, which contains a volume of liquid, e.g., sterile PBS sufficient to maintain the moisture content in the dish atmosphere, and the cells are cultured. AMDACs can also be cultured in standard or high-volume or high-throughput culture systems, such as T-flasks, Corning HYPERFLASK®, Cell Factories (Nuns), 1-, 2-, 4-, 10 or 40-Tray Cell stacks, and the like.

In one embodiment, amnion derived adherent cells are cultured in the presence of a compound that acts to maintain an undifferentiated phenotype in the cells. In a specific embodiment, the compound is a substituted 3,4-dihydropyridimol[4,5-d]pyrimidine. In a more specific embodiment, the compound is a compound having the following chemical structure:

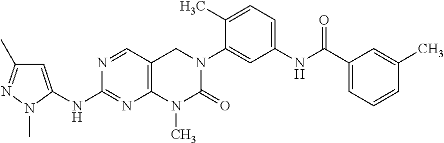

The compound can be contacted with an amnion derived adherent cell, or population of such cells, at a concentration of, for example, between about 1 μM to about 10 μM.

5.9.2 Expansion and Proliferation of Amnion Derived Adherent Cells

Once an isolated amnion derived adherent cell, or isolated population of such cells (e.g., amnion derived adherent cells, or population of such cells separated from at least 50% of the amnion cells with which the cell or population of cells is normally associated in vivo), the cells can be proliferated and expanded in vitro. For example, a population of adherent cells or amnion derived adherent cells can be cultured in tissue culture containers, e.g., dishes, flasks, multiwell plates, or the like, for a sufficient time for the cells to proliferate to 40-70% confluence, that is, until the cells and their progeny occupy 40-70% of the culturing surface area of the tissue culture container.

Amnion derived adherent cells can be seeded in culture vessels at a density that allows cell growth. For example, the cells may be seeded at low density (e.g., about 400 to about 6,000 cells/cm²) to high density (e.g., about 20,000 or more cells/cm²). In a preferred embodiment, the cells are cultured at about 0% to about 5% by volume CO₂ in air. In some preferred embodiments, the cells are cultured at about 0.1% to about 25% O₂ in air, preferably about 5% to about 20% O₂ in air. The cells are preferably cultured at about 25° C. to about 40° C., preferably at about 37° C.

The cells are preferably cultured in an incubator. During culture, the culture medium can be static or can be agitated, for example, during culture using a bioreactor. Amnion derived adherent cells preferably are grown under low oxidative stress (e.g., with addition of glutathione, ascorbic acid, catalase, tocopherol, N-acetylcysteine, or the like).

Although the amnion-derived adherent cells may be grown to confluence, the cells are preferably not grown to confluence. For example, once 40%-70% confluence is obtained, the cells may be passaged. For example, the cells can be enzymatically treated, e.g., trypsinized, using techniques well-known in the art, to separate them from the tissue culture surface. After removing the cells by pipetting and counting the cells, about 20,000-100,000 cells, preferably about 50,000 cells, or about 400 to about 6,000 cells/cm², can be passaged to a new culture container containing fresh culture medium. Typically, the new medium is the same type of medium from which the cells were removed. The amnion derived adherent cells can be passaged at least 1, 2, 3, 4, 5, 6, 7, 8, 9, 10, 11, 12, 13, 14, 15, 16, 17, 18, 19 or 20 times, or more. AMDACs can be doubled in culture at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or at least 50 times, or more.

### 5.10 Compositions Comprising Amnion Derived Adherent Cells

Further provided are pharmaceutical compositions that comprise the compositions described herein, and optionally a pharmaceutically-acceptable carrier.

In accordance with this embodiment, the combination compositions described herein may be formulated as an injectable (see, e.g., International Application No. WO 96/39101). In another embodiment, the composition comprising AMDACs and platelet rich plasma may be formulated using polymerizable or cross linking hydrogels as described, e.g., in U.S. Pat. Nos. 5,709,854; 5,516,532; 5,654,381.

In certain other embodiments, provided herein is the maintenance of each component of the composition, i.e., AMDACs and platelet rich plasma, respectively, prior to administration to an individual, as separate pharmaceutical compositions to be administered sequentially or jointly to create the combination composition in vivo. Each component may be stored and/or used in a separate container, e.g., a bag (e.g., blood storage bag from Baxter, Becton-Dickinson, Medcep, National Hospital Products, Terumo, etc.) or separate syringe, which contains a single type of cell or cell population. In a specific embodiment, platelet rich plasma is contained in one bag, and AMDACs, e.g., in suspension, are contained in a second bag.

The pharmaceutical compositions provided herein may comprise one or more agents that induce cell differentiation. In certain embodiments, an agent that induces differentiation includes, but is not limited to, Ca2+, epidermal growth factor (EGF), acidic fibroblast growth factor (aFGF), basic fibroblast growth factor (bFGF), platelet-derived growth factor (PDGF), keratinocyte growth factor (KGF), transforming growth factor beta (TGF-β), cytokines (e.g., interleukin-1 alpha (IL-1α), IL-1β, interferon gamma (IFN-γ), TFN), retinoic acid, transferrin, hormones (e.g., androgen, estrogen, insulin, prolactin, triiodothyroxine, hydrocortisone, dexamethasone), sodium butyrate, TPA, DMSO, NMF, DMF, matrix elements (e.g., collagen, laminin, heparan sulfate, MATRIGEL™), or combinations thereof.

In another embodiment, the pharmaceutical compositions provided herein may comprise one or more agents that suppress cellular differentiation. In certain embodiments, an agent that suppresses differentiation includes, but is not limited to, human Delta 1 and human Serrate 1 polypeptides (see, e.g., Sakano et al., U.S. Pat. No. 6,337,387), leukemia inhibitory factor (LIF), stem cell factor, or combinations thereof.

The pharmaceutical compositions provided herein may be treated prior to administration to an individual with a compound that modulates the activity of tumor necrosis factor-alpha (TNF-α). Such compounds are disclosed in detail in, e.g., U.S. Application Publication No. 2003/0235909, which disclosure is incorporated herein in its entirety. Preferred compounds are referred to as IMiDs (immunomodulatory compounds) and SeICIDs (Selective Cytokine Inhibitory Drugs), and particularly preferred compounds are available under the trade names ACTIMIDT™, REVIMID™ and REVLIMID™.

In certain embodiments, amnion derived adherent cells and platelet-rich plasma are contained within, or are components of, a pharmaceutical composition. The cells can be prepared in a form that is easily administrable to an individual, e.g., amnion derived adherent cells that are contained within a container that is suitable for medical use. Such a container can be, for example, a syringe, sterile plastic bag, flask, vial, jar, or other container from which the amnion derived adherent cell population can be easily dispensed. For example, the container can be a blood bag or other plastic, medically-acceptable bag suitable for the intravenous administration of a liquid to a recipient. The container, in certain embodiments, is one that allows for cryopreservation of the cells. The cells in the compositions, e.g., pharmaceutical compositions, provided herein, can comprise amnion derived adherent cells derived from a single donor, or from multiple donors. The cells can be completely HLA-matched to an intended recipient, or partially or completely HLA-mismatched.

Thus, in one embodiment, amnion derived adherent cells in the compositions provided herein are administered to an individual in need thereof in the form of a composition comprising amnion derived adherent cells in a container. In another specific embodiment, the container is a bag, flask, vial or jar. In more specific embodiment, said bag is a sterile plastic bag. In a more specific embodiment, said bag is suitable for, allows or facilitates intravenous administration of said adherent cells, e.g., by intravenous infusion, bolus injection, or the like. The bag can comprise multiple lumens or compartments that are interconnected to allow mixing of the cells and one or more other solutions, e.g., a drug, prior to, or during, administration. In another specific embodiment, prior to cryopreservation, the solution comprising the amnion derived adherent cells comprises one or more compounds that facilitate cryopreservation of the cells. In another specific embodiment, said amnion derived adherent cells are contained within a physiologically-acceptable aqueous solution. In a more specific embodiment, said physiologically-acceptable aqueous solution is a 0.9% NaCl solution. In another specific embodiment, said amnion derived adherent cells comprise cells that are HLA-matched to a recipient of said cells. In another specific embodiment, said amnion derived adherent cells comprise cells that are at least partially HLA-mismatched to a recipient of said cells. In another specific embodiment, said amnion derived adherent cells are derived from a plurality of donors. In various specific embodiments, said container comprises about, at least, or at most 1×10⁶ said cells, 5×10⁶ said cells, 1×10⁷ said stem cells, 5×10⁷ said cells, 1×10⁸ said cells, 5×10⁸ said cells, 1×10⁹ said cells, 5×10⁹ said cells, 1×10¹⁰, or 1×10¹¹ said cells. In other specific embodiments of any of the foregoing cryopreserved populations, said cells have been passaged about, at least, or no more than 5 times, no more than 10 times, no more than 15 times, or no more than 20 times. In another specific embodiment of any of the foregoing cryopreserved cells, said cells have been expanded within said container. In specific embodiments, a single unit dose of amnion derived adherent cells can comprise, in various embodiments, about, at least, or no more than 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more amnion derived adherent cells.

In certain embodiments, the pharmaceutical compositions provided herein comprises populations of amnion derived adherent cells, that comprise 50% viable cells or more (that is, at least 50% of the cells in the population are functional or living). Preferably, at least 60% of the cells in the population are viable. More preferably, at least 70%, 80%, 90%, 95%, or 99% of the cells in the population in the pharmaceutical composition are viable.

### 5.11 Preservation of Amnion Derived Adherent Cells

Amnion derived adherent cells can be preserved, that is, placed under conditions that allow for long-term storage, or conditions that inhibit cell death by, e.g., apoptosis or necrosis, e.g., during collection or prior to production of the compositions described herein, e.g., using the methods described herein.

Amnion derived adherent cells can be preserved using, e.g., a composition comprising an apoptosis inhibitor, necrosis inhibitor and/or an oxygen-carrying perfluorocarbon, as described in U.S. Application Publication No. 2007/0190042, the disclosure of which is hereby incorporated by reference in its entirety. In one embodiment, a method of preserving such cells, or a population of such cells, comprises contacting said cells or population of cells with a cell collection composition comprising an inhibitor of apoptosis and an oxygen-carrying perfluorocarbon, wherein said inhibitor of apoptosis is present in an amount and for a time sufficient to reduce or prevent apoptosis in the population of cells, as compared to a population of cells not contacted with the inhibitor of apoptosis. In a specific embodiment, said inhibitor of apoptosis is a caspase inhibitor. In another specific embodiment, said inhibitor of apoptosis is a JNK inhibitor. In a more specific embodiment, said JNK inhibitor does not modulate differentiation or proliferation of amnion derived adherent cells. In another embodiment, said cell collection composition comprises said inhibitor of apoptosis and said oxygen-carrying perfluorocarbon in separate phases. In another embodiment, said cell collection composition comprises said inhibitor of apoptosis and said oxygen-carrying perfluorocarbon in an emulsion. In another embodiment, the cell collection composition additionally comprises an emulsifier, e.g. lecithin. In another embodiment, said apoptosis inhibitor and said perfluorocarbon are between about 0° C. and about 25° C. at the time of contacting the cells. In another more specific embodiment, said apoptosis inhibitor and said perfluorocarbon are between about 2° C. and 10° C., or between about 2° C. and about 5° C., at the time of contacting the cells. In another more specific embodiment, said contacting is performed during transport of said population of cells. In another more specific embodiment, said contacting is performed during freezing and thawing of said population of cells.

Populations of amnion derived adherent cells can be preserved, e.g., by a method comprising contacting a population of said cells with an inhibitor of apoptosis and an organ-preserving compound, wherein said inhibitor of apoptosis is present in an amount and for a time sufficient to reduce or prevent apoptosis in the population of cells, as compared to a population of cells not contacted with the inhibitor of apoptosis. In a specific embodiment, the organ-preserving compound is UW solution (described in U.S. Pat. No. 4,798,824; also known as ViaSpan; see also Southard et al., Transplantation 49(2):251-257 (1990)) or a solution described in Stern et al., U.S. Pat. No. 5,552,267. In another embodiment, said organ-preserving compound is hydroxyethyl starch, lactobionic acid, raffinose, or a combination thereof. In another embodiment, the cell collection composition additionally comprises an oxygen-carrying perfluorocarbon, either in two phases or as an emulsion.

In another embodiment of the method, amnion derived adherent cells are contacted with a cell collection composition comprising an apoptosis inhibitor and oxygen-carrying perfluorocarbon, organ-preserving compound, or combination thereof, e.g., during a process of tissue disruption, e.g., enzymatic digestion of amnion tissue. In another embodiment, amnion derived adherent cells are contacted with said cell collection compound after collection by tissue disruption, e.g., enzymatic digestion of amnion tissue.

Typically, during collection of amnion derived adherent cells, enrichment and isolation, it is preferable to minimize or eliminate cell stress due to hypoxia and mechanical stress. In another embodiment of the method, therefore, an amnion derived adherent cell, or population of cells comprising the amnion derived adherent cells, is exposed to a hypoxic condition during collection, enrichment or isolation for less than six hours during said preservation, wherein a hypoxic condition is a concentration of oxygen that is, e.g., less than normal atmospheric oxygen concentration; less than normal blood oxygen concentration; or the like. In a more specific embodiment, said cells or population of said cells is exposed to said hypoxic condition for less than two hours during said preservation. In another more specific embodiment, said cells or population of said cells is exposed to said hypoxic condition for less than one hour, or less than thirty minutes, or is not exposed to a hypoxic condition, during collection, enrichment or isolation. In another specific embodiment, said population of cells is not exposed to shear stress during collection, enrichment or isolation.

Amnion derived adherent cells can be cryopreserved, in general or by the specific methods disclosed herein, e.g., in cryopreservation medium in small containers, e.g., ampoules. Suitable cryopreservation medium includes, but is not limited to, culture medium including, e.g., growth medium, or cell freezing medium, for example commercially available cell freezing medium, e.g., cell freezing medium identified by SigmaAldrich catalog numbers C2695, C2639 (Cell Freezing Medium-Serum-free 1×, not containing DMSO) or C6039 (Cell Freezing Medium-Glycoerol 1× containing Minimum Essential Medium, glycerol, calf serum and bovine serum), Lonza PROFREEZE™ 2× Medium, methylcellulose, dextran, human serum albumin, fetal bovine serum, fetal calf serum, or Plasmalyte. Cryopreservation medium preferably comprises DMSO (dimethylsulfoxide) or glycerol, at a concentration of, e.g., about 1% to about 20%, e.g., about 5% to 10% (v/v), optionally including fetal bovine serum or human serum. Cryopreservation medium may comprise additional agents, for example, methylcellulose with or without glycerol. Isolated amnion derived adherent cells are preferably cooled at about 1° C./min during cryopreservation. A preferred cryopreservation temperature is about −80° C. to about −180° C., preferably about −125° C. to about −140° C. Cryopreserved cells can be transferred to vapor phase of liquid nitrogen prior to thawing for use. In some embodiments, for example, once the ampoules have reached about −80° C., they are transferred to a liquid nitrogen storage area. Cryopreservation can also be done using a controlled-rate freezer. Cryopreserved cells preferably are thawed at a temperature of about 25° C. to about 40° C., preferably to a temperature of about 37° C.

### 5.12 Matrices Comprising Compositions

Further provided herein are matrices, hydrogels, scaffolds, and the like that comprise compositions comprising AMDACs and platelet rich plasma.

AMDACs or platelet rich plasma alone, e.g., prior to subsequent addition of the other component of the combination composition in vivo, or AMDACs combined with platelet rich plasma, can be seeded onto a natural matrix, e.g., a placental biomaterial such as an amniotic membrane material. Such an amniotic membrane material can be, e.g., amniotic membrane dissected directly from a mammalian placenta; fixed or heat-treated amniotic membrane, substantially dry (i.e., <20% H2O) amniotic membrane, chorionic membrane, substantially dry chorionic membrane, substantially dry amniotic and chorionic membrane, and the like. Preferred placental biomaterials on which the composition comprising AMDACs and platelet-rich plasma can be deposited or seeded are described, e.g., in Hariri, U.S. Application Publication No. 2004/0048796, the disclosure of which is hereby incorporated by reference in its entirety.

In certain embodiments, AMDACs or platelet rich plasma alone, e.g., prior to subsequent addition of the other component of the combination composition in vivo, or AMDACs combined with platelet rich plasma, can be suspended in a hydrogel solution suitable for, e.g., injection. Suitable hydrogels for such compositions include self-assembling peptides, such as RAD16. In one embodiment, a hydrogel solution comprising one or both of the components of the composition can be allowed to harden, for instance in a mold, to form a matrix for implantation. AMDACs in such a matrix can also be cultured so that the cells are mitotically expanded prior to implantation. The hydrogel can be, e.g., an organic polymer (natural or synthetic) that is cross-linked via covalent, ionic, or hydrogen bonds to create a three-dimensional open-lattice structure that entraps water molecules to form a gel. Hydrogel-forming materials include polysaccharides such as alginate and salts thereof, peptides, polyphosphazines, and polyacrylates, which are crosslinked ionically, or block polymers such as polyethylene oxide-polypropylene glycol block copolymers which are crosslinked by temperature or pH, respectively. In some embodiments, the hydrogel or matrix is biodegradable.

In some embodiments, the formulation comprises an in situ polymerizable gel (see., e.g., U.S. Patent Application Publication 2002/0022676; Anseth et al., J. Control Release, 78(1-3):199-209 (2002); Wang et al., Biomaterials, 24(22):3969-80 (2003).

In some embodiments, the polymers are at least partially soluble in aqueous solutions, such as water, buffered salt solutions, or aqueous alcohol solutions, that have charged side groups, or a monovalent ionic salt thereof. Examples of polymers having acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

AMDACs or platelet rich plasma alone, e.g. prior to subsequent addition of the other component of the combination composition in vivo, or AMDACs combined with platelet rich plasma, can be seeded onto a three-dimensional framework or scaffold and implanted in vivo. Such a framework can be implanted in combination with any one or more growth factors, cells, drugs or other components that stimulate tissue formation or otherwise enhance or improve the practice of the methods of treatment described elsewhere herein.

Examples of scaffolds that can be used in the methods of treatment described herein include nonwoven mats, porous foams, or self assembling peptides. Nonwoven mats can be formed using fibers comprised of a synthetic absorbable copolymer of glycolic and lactic acids (e.g., PGA/PLA) (VICRYL, Ethicon, Inc., Somerville, N.J.). Foams, composed of, e.g., poly(ε-caprolactone)/poly(glycolic acid) (PCL/PGA) copolymer, formed by processes such as freeze-drying, or lyophilization (see, e.g., U.S. Pat. No. 6,355,699), can also be used as scaffolds. Other scaffolds may comprise oxidized cellulose or oxidized regenerated cellulose.

In another embodiment, the scaffold is, or comprises, a nanofibrous scaffold, e.g., an electrospun nanofibrous scaffold. In a more specific embodiment, said nanofibrous scaffold comprises poly(L-lactic acid) (PLLA), type I collagen, a copolymer of vinylidene fluoride and trifluoroethylnee (PVDF-TrFE), poly(-caprolactone), poly(L-lactide-co-ε-caprolactone) [P(LLA-CL)] (e.g., 75:25), and/or a copolymer of poly(3-hydroxybutyrate-co-3-hydroxyvalerate) (PHBV) and type I collagen. In another more specific embodiment, said scaffold promotes the differentiation of placental cells into chondrocytes. Methods of producing nanofibrous scaffolds, e.g., electrospun nanofibrous scaffolds, are known in the art. See, e.g., Xu et al., Tissue Engineering 10(7):1160-1168 (2004); Xu et al., Biomaterials 25:877-886 (20040; Meng et al., J. Biomaterials Sci., Polymer Edition 18(1):81-94 (2007).

AMDACs or platelet rich plasma alone, e.g., prior to subsequent addition of the other component of the combination composition in vivo, or AMDACs combined with platelet rich plasma, can also be seeded onto, or contacted with, a physiologically-acceptable ceramic material including, but not limited to, mono-, di-, tri-, alpha-tri-, beta-tri-, and tetra-calcium phosphate, hydroxyapatite, fluoroapatites, calcium sulfates, calcium fluorides, calcium oxides, calcium carbonates, magnesium calcium phosphates, biologically active glasses such as BIOGLASS®, and mixtures thereof. Porous biocompatible ceramic materials currently commercially available include SURGIBONE® (CanMedica Corp., Canada), ENDOBON® (Merck Biomaterial France, France). CEROS® (Mathys, A G, Bettlach, Switzerland), and mineralized collagen bone grafting products such as HEALOS™ (DePuy. Inc., Raynham, Mass.) and VITOSS®, RHAKOSS™, and CORTOSS® (Orthovita, Malvern, Pa.). The framework can be a mixture, blend or composite of natural and/or synthetic materials.

In another embodiment, AMDACs or platelet rich plasma alone, e.g., prior to subsequent addition of the other component of the composition in vivo, or the composition comprising AMDACs combined with platelet rich plasma, can be seeded onto, or contacted with, a felt, which can be, e.g., composed of a multifilament yarn made from a bioabsorbable material such as PGA, PLA, PCL copolymers or blends, or hyaluronic acid.

AMDACs or platelet rich plasma alone, e.g., prior to subsequent addition of the other component of the combination composition in vivo, or the composition comprising AMDACs combined with platelet rich plasma, can, in another embodiment, be seeded onto foam scaffolds that may be composite structures. Such foam scaffolds can be molded into a useful shape, such as that of a portion of a specific structure in the body to be repaired, replaced or augmented. In some embodiments, the framework is treated, e.g., with 0.1M acetic acid followed by incubation in polylysine, PBS, and/or collagen, prior to inoculation of the immunosuppressive placental cells in order to enhance cell attachment. External surfaces of a matrix may be modified to improve the attachment or growth of cells and differentiation of tissue, such as by plasma-coating the matrix, or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate, etc.), a cellular matrix, and/or other materials such as, but not limited to, gelatin, alginates, agar, agarose, and plant gums, and the like.

In some embodiments, the scaffold comprises, or is treated with, materials that render it non-thrombogenic. These treatments and materials may also promote and sustain endothelial growth, migration, and extracellular matrix deposition. Examples of these materials and treatments include but are not limited to natural materials such as basement membrane proteins such as laminin and Type IV collagen, synthetic materials such as EPTFE, and segmented polyurethaneurea silicones, such as PURSPAN™ (The Polymer Technology Group, Inc., Berkeley, Calif.). The scaffold can also comprise anti-thrombotic agents such as heparin; the scaffolds can also be treated to alter the surface charge (e.g., coating with plasma) prior to seeding with AMDACs or the composition comprising AMDACs and platelet-rich plasma.

In particular embodiments, the combination compositions comprising AMDACs and platelet rich plasma provided herein are not seeded on a matrix, a hydrogel, a scaffold, or the like prior to transplantation in an individual in need of said combination composition. In another particular embodiment, the combination compositions do not comprise an implantable bone substitute when transplanted in an individual in need of said combination composition.

### 5.13 Modified Amnion Derived Adherent Cells

5.13.1 Genetically Modified Amnion Derived Adherent Cells

In another aspect, the amnion derived adherent cells described herein can be genetically modified, e.g., to produce a nucleic acid or polypeptide of interest, or to produce a differentiated cell, e.g., an osteogenic cell, myocytic cell, pericytic cell, or angiogenic cell, that produces a nucleic acid or polypeptide of interest. Genetic modification can be accomplished, e.g., using virus-based vectors including, but not limited to, non-integrating replicating vectors, e.g., papilloma virus vectors, SV40 vectors, adenoviral vectors; integrating viral vectors, e.g., retrovirus vector or adeno-associated viral vectors; or replication-defective viral vectors. Other methods of introducing DNA into cells include the use of liposomes, electroporation, a particle gun, direct DNA injection, or the like.

The adherent cells provided herein can be, e.g., transformed or transfected with DNA controlled by or in operative association with, one or more appropriate expression control elements, for example, promoter or enhancer sequences, transcription terminators, polyadenylation sites, internal ribosomal entry sites. Preferably, such a DNA incorporates a selectable marker. Following the introduction of the foreign DNA, engineered adherent cells can be, e.g., grown in enriched media and then switched to selective media. In one embodiment, the DNA used to engineer an amnion derived adherent cell comprises a nucleotide sequence encoding a polypeptide of interest, e.g., a cytokine, growth factor, differentiation agent, or therapeutic polypeptide.

The DNA used to engineer the adherent cell can comprise any promoter known in the art to drive expression of a nucleotide sequence in mammalian cells, e.g., human cells. For example, promoters include, but are not limited to, CMV promoter/enhancer, SV40 promoter, papillomavirus promoter, Epstein-Barr virus promoter, elastin gene promoter, and the like. In a specific embodiment, the promoter is regulatable so that the nucleotide sequence is expressed only when desired. Promoters can be either inducible (e.g., those associated with metallothionein and heat shock proteins) or constitutive.

In another specific embodiment, the promoter is tissue-specific or exhibits tissue specificity. Examples of such promoters include but are not limited to: myelin basic protein gene control region (Readhead et al., 1987, Cell 48:703) (oligodendrocyte cells): elastase I gene control region (Swit et al., 1984, Cell 38:639; Ornitz et al., 1986, Cold Spring Harbor Symp. Quant. Biol. 50:399; MacDonald, 1987, Hepatology 7:425) (pancreatic acinar cells); insulin gene control region (Hanahan, 1985, Nature 315:115) (pancreatic beta cells); myosin light chain-2 gene control region (Shani, 1985, Nature 314:283) (skeletal muscle).

The amnion derived adherent cells disclosed herein may be engineered or otherwise selected to “knock out” or “knock down” expression of one or more genes in such cells. The expression of a gene native to a cell can be diminished by, for example, inhibition of expression by inactivating the gene completely by, e.g., homologous recombination. In one embodiment, for example, an exon encoding an important region of the protein, or an exon 5′ to that region, is interrupted by a positive selectable marker, e.g., neo, preventing the production of normal mRNA from the target gene and resulting in inactivation of the gene. A gene may also be inactivated by creating a deletion in part of a gene or by deleting the entire gene. By using a construct with two regions of homology to the target gene that are far apart in the genome, the sequences intervening the two regions can be deleted (Mombaerts et al., 1991, Proc. Nat. Acad. Sci. U.S.A. 88:3084). Antisense, morpholinos, DNAzymes, small interfering RNA, short hairpin RNA, and ribozyme molecules that inhibit expression of the target gene can also be used to reduce the level of target gene activity in the adherent cells. For example, antisense RNA molecules which inhibit the expression of major histocompatibility gene complexes (HLA) have been shown to be most versatile with respect to immune responses. Triple helix molecules can be utilized in reducing the level of target gene activity. See, e.g., L. G. Davis et al. (eds), 1994, BASIC METHODS IN MOLECULAR BIOLOGY, 2nd ed., Appleton & Lange, Norwalk, Conn., which is incorporated herein by reference.

In a specific embodiment, the amnion derived adherent cells disclosed herein can be genetically modified with a nucleic acid molecule comprising a nucleotide sequence encoding a polypeptide of interest, wherein expression of the polypeptide of interest is controllable by an exogenous factor, e.g., polypeptide, small organic molecule, or the like. The polypeptide of interest can be a therapeutic polypeptide. In a more specific embodiment, the polypeptide of interest is IL-12 or interleukin-1 receptor antagonist (IL-1Ra). In another more specific embodiment, the polypeptide of interest is a fusion of interleukin-1 receptor antagonist and dihydrofolate reductase (DHFR), and the exogenous factor is an antifolate, e.g. methotrexate. Such a construct is useful in the engineering of amnion derived adherent cells that express IL-1Ra, or a fusion of IL-1Ra and DHFR, upon contact with methotrexate. Such a construct can be used, e.g., in the treatment of rheumatoid arthritis. In this embodiment, the fusion of IL-1Ra and DHFR is translationally upregulated upon exposure to an antifolate such as methotrexate. Therefore, in another specific embodiment, the nucleic acid used to genetically engineer an amnion derived adherent cell can comprise nucleotide sequences encoding a first polypeptide and a second polypeptide, wherein said first and second polypeptides are expressed as a fusion protein that is translationally upregulated in the presence of an exogenous factor. The polypeptide can be expressed transiently or long-term (e.g., over the course of weeks or months). Such a nucleic acid molecule can additionally comprise a nucleotide sequence encoding a polypeptide that allows for positive selection of engineered cells, or allows for visualization of the engineered cells. In another more specific embodiment, the nucleotide sequence encodes a polypeptide that is, e.g., fluorescent under appropriate visualization conditions, e.g., luciferase (Luc). In a more specific embodiment, such a nucleic acid molecule can comprise IL-1Ra-DHFR-IRES-Luc, where IL-1Ra is interleukin-1 receptor antagonist, IRES is an internal ribosomal entry site, and DHFR is dihydrofolate reductase.

5.13.2 Immortalized Amnion Derived Adherent Cell Lines

Mammalian amnion derived adherent cells can be conditionally immortalized by transfection with any suitable vector containing a growth-promoting gene, that is, a gene encoding a protein that, under appropriate conditions, promotes growth of the transfected cell, such that the production and/or activity of the growth-promoting protein is regulatable by an external factor. In a preferred embodiment the growth-promoting gene is an oncogene such as, but not limited to, v-myc, N-myc, c-myc, p53, SV40 large T antigen, polyoma large T antigen, E1a adenovirus or E7 protein of human papillomavirus. In another embodiment, amnion derived adherent cells can be immortalized using cre-lox recombination, as exemplified for a human pancreatic β-cell line by Narushima, M., et al (Nature Biotechnology, 2005, 23(10:1274-1282).

External regulation of the growth-promoting protein can be achieved by placing the growth-promoting gene under the control of an externally-regulatable promoter, e.g., a promoter the activity of which can be controlled by, for example, modifying the temperature of the transfected cells or the composition of the medium in contact with the cells, in one embodiment, a tetracycline (tet)-controlled gene expression system can be employed (see Gossen et al., Proc. Natl. Acad. Sci. USA 89:5547-5551, 1992; Hoshimaru et al., Proc. Natl. Acad. Sci. USA 93:1518-1523, 1996). In the absence of tet, a tet-controlled transactivator (tTA) within this vector strongly activates transcription from ph_{CMV*-1}, a minimal promoter from human cytomegalovirus fused to tet operator sequences. tTA is a fusion protein of the repressor (tetR) of the transposon-10-derived tet resistance operon of Escherichia coli and the acidic domain of VP16 of herpes simplex virus. Low, non-toxic concentrations of tet (e.g., 0.01-1.0 μg/mL) almost completely abolish transactivation by tTA.

In one embodiment, the vector further contains a gene encoding a selectable marker, e.g., a protein that confers drug resistance. The bacterial neomycin resistance gene (neo^{R}) is one such marker that may be employed within the present methods. Cells carrying neo^{R} may be selected by means known to those of ordinary skill in the art, such as the addition of, e.g., 100-200 μg/mL G418 to the growth medium.

Transfection can be achieved by any of a variety of means known to those of ordinary skill in the art including, but not limited to, retroviral infection. In general, a cell culture may be transfected by incubation with a mixture of conditioned medium collected from the producer cell line for the vector and DMEM/F12 containing N2 supplements. For example, a placental cell culture prepared as described above may be infected after, e.g., five days in vitro by incubation for about 20 hours in one volume of conditioned medium and two volumes of DMEM/F12 containing N2 supplements. Transfected cells carrying a selectable marker may then be selected as described above.

Following transfection, cultures are passaged onto a surface that permits proliferation, e.g., allows at least 30% of the cells to double in a 24 hour period. Preferably, the substrate is a polyornithine/laminin substrate, consisting of tissue culture plastic coated with polyornithine (10 μg/mL) and/or laminin (10 μg/mL), a polylysine/laminin substrate or a surface treated with fibronectin. Cultures are then fed every 3-4 days with growth medium, which may or may not be supplemented with one or more proliferation-enhancing factors. Proliferation-enhancing factors may be added to the growth medium when cultures are less than 50% confluent.

The conditionally-immortalized amnion derived adherent cell lines can be passaged using standard techniques, such as by trypsinization, when 80-95% confluent. Up to approximately the twentieth passage, it is, in some embodiments, beneficial to maintain selection (by, for example, the addition of G418 for cells containing a neomycin resistance gene). Cells may also be frozen in liquid nitrogen for long-term storage.

Clonal cell lines can be isolated from a conditionally-immortalized adherent cell line prepared as described above. In general, such clonal cell lines may be isolated using standard techniques, such as by limit dilution or using cloning rings, and expanded. Clonal cell lines may generally be fed and passaged as described above.

Conditionally-immortalized human amnion derived adherent cells lines, which may, but need not, be clonal, may generally be induced to differentiate by suppressing the production and/or activity of the growth-promoting protein under culture conditions that facilitate differentiation. For example, if the gene encoding the growth-promoting protein is under the control of an externally-regulatable promoter, the conditions, e.g., temperature or composition of medium, may be modified to suppress transcription of the growth-promoting gene. For the tetracycline-controlled gene expression system discussed above, differentiation can be achieved by the addition of tetracycline to suppress transcription of the growth-promoting gene. In general, 1 μg/mL tetracycline for 4-5 days is sufficient to initiate differentiation. To promote further differentiation, additional agents may be included in the growth medium.

### 5.14 Dosages and Routes of Administration

Administration of amnion derived adherent cells (AMDACs) to an individual in need thereof can be by any medically-acceptable route relevant for the immune-related disease or condition to be treated. In another specific embodiment of the methods of treatment described above, said AMDACs are administered by bolus injection. In another specific embodiment, said isolated AMDACs are administered intravenously, e.g., by intravenous infusion. In a specific embodiment, said intravenous infusion is intravenous infusion over about 1 to about 8 hours. In another specific embodiment, said isolated AMDACs are administered locally, e.g., at a particular site in the body of the individual that is affected by the immune-related disease or condition. In another specific embodiment, said isolated AMDACs are administered intracranially. In another specific embodiment, said isolated AMDACs are administered intramuscularly. In another specific embodiment, said isolated AMDACs are administered intraperitoneally. In another specific embodiment, said isolated AMDACs are administered intra-arterially. In another specific embodiment of the method of treatment, said isolated AMDACs are administered intramuscularly, intradermally, or subcutaneously. In another specific embodiment, said isolated AMDACs are administered intravenously. In another specific embodiment, said isolated AMDACs are administered intraventricularly. In another specific embodiment, said isolated AMDACs are administered intrasternally. In another specific embodiment, said isolated AMDACs are administered intrasynovially. In another specific embodiment, said isolated AMDACs are administered intraocularly. In another specific embodiment, said isolated AMDACs are administered intravitreally. In another specific embodiment, said isolated AMDACs are administered intracerebrally. In another specific embodiment, said isolated AMDACs are administered intracerebroventricularly. In another specific embodiment, said isolated AMDACs are administered intrathecally. In another specific embodiment, said isolated AMDACs are administered by intraosseous infusion. In another specific embodiment, said isolated AMDACs are administered intravesically. In another specific embodiment, said isolated AMDACs are administered transdermally. In another specific embodiment, said isolated AMDACs are administered intracisternally. In another specific embodiment, said isolated AMDACs are administered epidurally.

In another specific embodiment of the methods of treatment described above, said AMDACs are administered once to said individual. In another specific embodiment, said isolated AMDACs are administered to said individual in two or more separate administrations. In another specific embodiment, said administering comprises administering between about 1×10⁴ and 1×10⁵ isolated AMDACs, e.g., AMDACs per kilogram of said individual. In another specific embodiment, said administering comprises administering between about 1×10⁵ and 1×10⁶ isolated AMDACs per kilogram of said individual. In another specific embodiment, said administering comprises administering between about 1×10⁶ and 1×10⁷ isolated AMDACs per kilogram of said individual. In another specific embodiment, said administering comprises administering between about 1×10⁷ and 1×10⁸ isolated AMDACs per kilogram of said individual. In another specific embodiment, said administering comprises administering between about 1×10⁸ and 1×10⁹ isolated AMDACs per kilogram of said individual. In another specific embodiment, said administering comprises administering between about 1×10⁹ and 1×10¹⁰ isolated AMDACs per kilogram of said individual. In another specific embodiment, said administering comprises administering between about 1×10¹⁰ and 1×10¹¹ isolated AMDACs per kilogram of said individual.

In other specific embodiments, said administering comprises administering between about 1×10⁶ and about 2×10⁶ isolated AMDACs per kilogram of said individual; between about 2×10⁶ and about 3×10⁶ isolated AMDACs per kilogram of said individual; between about 3×10⁶ and about 4×10⁶ isolated AMDACs per kilogram of said individual; between about 4×10⁶ and about 5×10⁶ isolated AMDACs per kilogram of said individual; between about 5×10⁶ and about 6×10⁶ isolated AMDACs per kilogram of said individual; between about 6×10⁶ and about 7×10⁶ isolated AMDACs per kilogram of said individual; between about 7×10⁶ and about 8×10⁶ isolated AMDACs per kilogram of said individual; between about 8×10⁶ and about 9×10⁶ isolated AMDACs per kilogram of said individual; or between about 9×10⁶ and about 1×10⁷ isolated AMDACs per kilogram of said individual. In another specific embodiment, said administering comprises administering between about 1×10⁷ and about 2×10⁷ isolated AMDACs per kilogram of said individual to said individual. In another specific embodiment, said administering comprises administering between about 1.3×10⁷ and about 1.5×10⁷ isolated AMDACs per kilogram of said individual to said individual. In another specific embodiment, said administering comprises administering up to about 3×10⁷ isolated AMDACs per kilogram of said individual to said individual. In a specific embodiment, said administering comprises administering between about 5×10⁶ and about 2×10⁷ isolated AMDACs to said individual. In another specific embodiment, said administering comprises administering about 150×10⁶ isolated AMDACs in about 20 milliliters of solution to said individual.

In another specific embodiment of the methods of treatment described above, isolated AMDACs are administered to an individual as a single unit dose. In specific embodiments, a single unit dose of AMDACs can comprise, in various embodiments, about, at least, or no more than 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more AMDACs.

In a specific embodiment, said administering comprises administering between about 5×10⁶ and about 2×10⁷ isolated AMDACs to said individual, wherein said cells are contained in a solution comprising 10% dextran, e.g., dextran-40, 5% human serum albumin, and optionally an immunosuppressant. In another specific embodiment, said administering comprises administering between about 5×10⁷ and 3×10⁹ isolated AMDACs intravenously. In more specific embodiments, said administering comprises administering about 9×10⁸ isolated AMDACs or about 1.8×10⁹ isolated AMDACs intravenously. In another specific embodiment, said administering comprises administering between about 5×10⁷ and 1×10⁸ isolated AMDACs intracranially. In a more specific embodiment, said administering comprises administering about 9×10⁷ isolated AMDACs intracranially.

Administration of medium conditioned by AMDACs to an individual in need thereof can be by any medically-acceptable route relevant for the disease, disorder or condition associated with CNS injury to be treated including, but not limited to bolus injection, intravenously (e.g., by intravenous infusion), locally (e.g., at a particular site in the body of the individual that is affected by the disease, disorder or condition associated with CNS injury), intracranially, intramuscularly, intraperitoneally, intra-arterially, intramuscularly, intradermally, subcutaneously, intraventricularly, intrasynovially, intraocularly, intravitreally, intracerebrally, intracerebroventricularly, intrathecally, by intraosseous infusion, intravesically, transdermally, intracisternally, or epidurally. In a specific embodiment, the medium conditioned by AMDACs is administered by continuous infusion. In another specific embodiment, the medium conditioned by AMDACs is administered as a single dose.

In some embodiments, administration of medium conditioned by AMDACs to an individual in need thereof comprises administering about 0.01 to about 0.02 ml of medium conditioned by AMDACs per 100 grams of body weight, about 0.01 to about 0.05 ml of medium conditioned by AMDACs per 100 grams of body weight, about 0.01 to about 0.1 ml of medium conditioned by AMDACs per 100 grams of body weight, about 0.01 to about 0.15 ml of medium conditioned by AMDACs per 100 grams of body weight, about 0.01 to about 0.2 ml of medium conditioned by AMDACs per 100 grams of body weight, about 0.01 to about 0.25 ml of medium conditioned by AMDACs per 100 grams of body weight, about 0.01 to about 0.3 ml of medium conditioned by AMDACs per 100 grams of body weight, about 0.01 to about 0.35 ml of medium conditioned by AMDACs per 100 grams of body weight, about 0.01 to about 0.4 ml of medium conditioned by AMDACs per 100 grams of body weight, about 0.01 to about 0.45 ml of medium conditioned by AMDACs per 100 grams of body weight, or about 0.01 to about 0.5 ml of medium conditioned by AMDACs per 100 grams of body weight.

### 5.15 Differentiation of Amnion Derived Adherent Cells

The amnion derived adherent cells provided herein can be differentiated. In one embodiment, the cell has been differentiated sufficiently for said cell to exhibit at least one characteristic of an endothelial cell, a myogenic cell, or a pericytic cell, e.g., by contacting the cell with vascular endothelial growth factor (VEGF), or as described in Sections 5.11.2, 6.3.3, or 6.3.4, below. In more specific embodiments, said characteristic of an endothelial cell, myogenic cell or pericytic cell is expression of one or more of CD9, CD31, CD54, CD102, NG2 (neural/glial antigen 2) or alpha smooth muscle actin, which is increased compared to an amniotic cell that is OCT-4⁻, VEGFR2/KDR⁺, CD9⁺, CD54⁺, CD105⁺, CD200⁺, and VE-cadherin⁻. In other more specific embodiments, said characteristic of an endothelial cell, myogenic cell or pericytic cell is expression of one or more of CD9, CD31, CD54, CD102, NG2 (neural/glial antigen 2) or alpha smooth muscle actin, which is increased compared to an amniotic cell that is OCT-4⁻, VEGFR2/KDR⁺, and VEGFR1/Flt-1⁺.

Myogenic (cardiogenic) differentiation of the amnion derived adherent cells provided herein can be accomplished, for example, by placing the cells in cell culture conditions that induce differentiation into cardiomyocytes. A preferred cardiomyocytic medium comprises DMEM/20% CBS supplemented with retinoic acid, 1 μM; basic fibroblast growth factor, 10 ng/mL; and transforming growth factor beta-1, 2 ng/mL; and epidermal growth factor, 100 ng/mL. KnockOut Serum Replacement (Invitrogen, Carlsbad, Calif.) may be used in lieu of CBS. Alternatively, the amnion derived adherent cells are cultured in DMEM/20% CBS supplemented with 1 to 100, e.g., 50 ng/mL Cardiotropin-1 for 24 hours. In another embodiment, amnion derived adherent cells can be cultured 10-14 days in protein-free medium for 5-7 days, then stimulated with human myocardium extract, e.g., produced by homogenizing human myocardium in 1% HEPES buffer supplemented with 1% cord blood serum.

Differentiation can be confirmed by demonstration of cardiac actin gene expression, e.g., by RT/PCR, or by visible beating of the cell. An adherent cell is considered to have differentiated into a cardiac cell when the cell displays one or more of these characteristics.

5.15.1 Differentiation into Neurogenic Cells

Amnion derived angiogenic cells, when cultured under neurogenic conditions, differentiate into cells displaying neural morphology and neural markers. For example, AMDACs, e.g., AMDACs expanded for 4 days in DMEM/F12 medium containing 15% v/v FBS, with basic fibroblast growth factor (bFGF), e.g., at about 20 ng/ml, epidermal growth factor (EGF), e.g., at about 20 ng/ml, e.g., for four days, followed by culture for four days in induction medium comprising DMEM/F12, serum free, containing 200 mM butylated hydroxyanisole, 10 nM potassium chloride, 5 mgs/mL insulin, 10 nM forskolin, 4 nM valproic acid, and 2 nM hydrocortisone. Under these conditions, AMDACs display expression of human nestin, Tuj1 and GFAP, as assessed by antibody staining.

5.15.2 Non-Differentiation into Osteogenic Cells

Amnion derived adherent cells do not show osteogenic differentiation in standard assays for osteogenesis. For example, in one embodiment, lack of osteogenic differentiation by AMDACs can be shown, e.g., by lack of deposition of calcium, as shown by lack of von Kossa staining of AMDACs under osteogenic conditions. For example, AMDACs, e.g., freshly-prepared or cryopreserved AMDACs, can be suspended in growth medium, e.g., at about 5000 cells/cm² in 24-well plates and 6-well plates in growth medium and incubated overnight, then cultured for 14-35 days, e.g., 28, days in osteogenic medium. In certain embodiments, osteogenic medium comprises DMEM-low glucose, 10% v/v fetal bovine serum (FBS), 10 mM beta glycerophosphate, 100 nM dexamethasone, and 100 μM ascorbic acid phosphate salt supplemented with transforming growth factor-beta1 (TGF-β1), e.g., at 1-100 ng/mL, e.g., 20 ng/mL, and human recombinant bone morphogenetic protein-2 (BMP-2) at, e.g., 1-100 ng/mL, e.g., 40 ng/mL. Cells are then stained using von Kossa stain using standard protocols; development of black silver deposits indicates the presence of mineralization. In the case of AMDACs, cultures should be substantially, e.g., completely, free of deposits, e.g., as compared to bone marrow-mesenchymal stem cells, indicating that the AMDACs do not produce calcium deposits, and therefore do not differentiate down an osteogenic pathway.

5.15.3 Non-Differentiation into Chondrogenic Cells

Amnion derived adherent cells similarly do not show chondrogenic differentiation in standard assays for chondrogenesis. For example, in one embodiment, lack of chondrogenic differentiation by AMDACs can be shown, e.g., by lack of development by AMDACs of cell pellets in a chondrogenesis assay in which chondrogenic cells for cell pellets. For example, AMDACs, e.g., freshly prepared or cryopreserved, e.g., 2.5×10⁵ cells, can be placed in 15 mL conical tubes and centrifuged at 200×g for 5 minutes at room temperature to form a spherical cell pellet. The collected cells are then cultured in chondrogenic induction medium, e.g., Lonza Chondrocyte Medium containing TGF beta-3 (e.g., at about 10 ng/mL), recombinant human growth/differentiation factor-5 (rhGDF-5) (e.g., at about 500 ng/mL), or a combination of TGF beta-3 (10 nanogram/milliliter), and rhGDF-5 (e.g., at about 500 ng/mL) for three weeks. At the end of three weeks, the cells are stained with Alcian blue, which stains for mucopolysaccharides and glycosaminoglycans that are produced by chondrogenic cells. Typically, while BM-MSCs or chondrocytes will, when cultured under these conditions, develop cell pellets that stain positively for Alcian blue, AMDACs neither form pellets nor stain with Alcian blue.

### 6. EXAMPLES

### 6.1 Example 1

### Isolation and Expansion of Adherent Cells from Amniotic Membrane

This Example demonstrates the isolation and expansion of amnion derived adherent cells.

6.1.1 Isolation

Amnion derived adherent cells were isolated from amniotic membrane as follows. Amnion/chorion were cut from the placenta, and amnion was manually separated from the chorion. The amnion was rinsed with sterile PBS to remove residual blood, blood clots and other material. Sterile gauze was used to remove additional blood, blood clots or other material that was not removed by rinsing, and the amnion was rinsed again with PBS. Excess PBS was removed from the membrane, and the amnion was cut with a scalpel into 2″ by 2″ segments. For epithelial cell release, a processing vessel was set up by connecting a sterile jacketed glass processing vessel to a circulating 37° C. water bath using tubing and connectors, and set on a stir plate. Trypsin (0.25%, 300 mL) was warmed to 37° C. in the processing vessel; the amnion segments were added, and the amnion/trypsin suspension was agitated, e.g., at 100 RPM-150 RPM at 37° C. for 15 minutes. A sterile screening system was assembled by placing a sterile receptacle on a sterile field next to the processing vessel and inserting a sterile 75 μm to 125 μm screen into the receptacle (Millipore, Billerica, Mass.). After agitating the amnion segments for 15 minutes, the contents of the processing vessel were transferred to the screen, and the amnion segments were transferred, e.g., using sterile tweezers back into the processing vessel; the trypsin solution containing the epithelial cells was discarded. The amnion segments were agitated again with 300 mL trypsin solution (0.25%) as described above. The screen was rinsed with approximately 100-150 mL of PBS, and the PBS solution was discarded. After agitating the amnion segments for 15 minutes, the contents of the processing vessel were transferred to the screen. The amnion segments were then transferred back into the processing vessel; the trypsin solution containing the epithelial cells was discarded. The amnion segments were agitated again with 300 mL trypsin solution (0.25%) as described above. The screen was rinsed with approximately 100-150 mL of PBS, and the PBS solution was discarded. After agitating the amnion segments for 15 minutes, the contents of the processing vessel were transferred to the screen. The amnion segments were then transferred back into the processing vessel, and the trypsin solution containing the epithelial cells was discarded. The amnion segments were agitated in PBS/5% FBS (1:1 ratio of amnion to PBS/5% FBS solution by volume) at 37° C. for approximately 2-5 minutes to neutralize the trypsin. A fresh sterile screen system was assembled. After neutralizing the trypsin, the contents of the processing vessel were transferred to the new screen, and the amnion segments were transferred back into the processing vessel. Room temperature, sterile PBS (400 mL) was added to the processing vessel, and the contents of the processing vessel were agitated for approximately 2-5 minutes. The screen was rinsed with approximately 100-150 mL of PBS. After agitation, the contents of the processing vessel were transferred to the screen; the processing flask was rinsed with PBS, and the PBS solution was discarded. The processing vessel was then filled with 300 mL of pre-warmed DMEM, and the amnion segments were transferred into the DMEM solution.

For release of the amnion derived adherent cells, the treated amniotic membrane was further treated with collagenase as follows. A sterile collagenase stock solution (500 U/mL) was prepared by dissolving the appropriate amount of collagenase powder (varied with the activity of the collagenase lot received from the supplier) in DMEM. The solution was filtered through a 0.22 μm filter and dispensed into individual sterile containers. CaCl₂ solution (0.5 mL, 600 mM) was added to each 100 mL dose, and the doses were frozen. Collagenase (100 mL) was added to the amnion segments in the processing vessel, and the processing vessel was agitated for 30-50 minutes, or until amnion digestion was complete by visual inspection. After amnion digestion was complete, 100 mL of pre-warmed sterile PBS/5% FBS was added to the processing vessel, and the processing vessel was agitated for an additional 2-3 minutes. Following agitation, the contents of the flask were transferred to a sterile 60 μm screen, and the liquid was collected by vacuum filtration. The processing vessel was rinsed with 400 mL of PBS, and the PBS solution was sterile-filtered. The filtered cell suspension was then centrifuged at 300×g for 15 minutes at 20° C., and the cell pellets were resuspended in pre-warmed PBS/2% FBS (approximately 10 mL total).

6.1.2 Establishment

Freshly isolated angiogenic amniotic cells were added to growth medium containing 60% DMEM-LG (Gibco); 40% MCBD-201 (Sigma); 2% FBS (Hyclone Labs), 1× insulin-transferrin-selenium (ITS); 10 ng/mL linoleic acid-bovine serum albumin (LA-BSA); 1 n-dexamethasone (Sigma); 100 μM ascorbic acid 2-phosphate (Sigma); 10 ng/mL epidermal growth factor (R & D Systems); and 10 ng/mL platelet-derived growth factor (PDGF-BB) (R & D Systems) and were plated in a T-Flask at a seeding density of 10,000 cells per cm². The culture device(s) were then incubated at 37° C., 5% CO₂ with >90% humidity. Cellular attachment, growth, and morphology were monitored daily. Non-adherent cells and debris were removed by medium exchange. Medium exchange was performed twice per week. Adherent cells with typical fibroblastoid/spindle shape morphology appeared at several days after initial plating. When confluency reached 40%-70% (at 4-11 days after initial plating), the cells were harvested by trypsinization (0.25% trypsin—EDTA) for 5 minutes at room temperature (37° C.). After neutralization with PBS-5% FBS, the cells were centrifuged at 200-400 g for 5-15 minutes at room temperature, and then were resuspended in growth medium. At this point, an AMDAC line was considered to be successfully established at the initial passage. Initial passage amnion derived adherent cells were, in some cases, cryopreserved or expanded (e.g., grown in culture such that the number of cells increases).

6.1.3 Culture Procedure

Amnion derived adherent cells were cultured in the growth medium described above and seeded at a density of 2000-4000 per cm² in an appropriate tissue culture-treated culture device(s). The culture device(s) were then incubated at 37° C., 5% CO₂ with >90% humidity. During culture, AMDACs would adhere and proliferate. Cellular growth, morphology, and confluency were monitored daily. Medium exchange was performed twice a week to replenish fresh nutrients if the culture extended to 5 days or more. When confluency reached 40%-70% (at 3-7 days after seeding), the cells were harvested by trypsinization (0.05%-0.25% trypsin—EDTA) for 5 minutes at room temperature (37° C.). After neutralization with PBS-5% FBS, the cells were centrifuged at 200-400 g for 5-15 minutes at room temperature, then were resuspended in growth medium.

AMDACs isolated and cultured in this manner typically produced 33530+/−15090 colony-forming units (fibroblast) (CFU-F) out of 1×10⁶ cells plated.

### 6.2 Example 2

### Phenotypic Characterization of Amnion Derived Adherent Cells

6.2.1 Gene and Protein Expression Profiles

This Example describes phenotypic characterization of amnion derived adherent cells, including characteristic cell surface marker, mRNA, and proteomic expression.

Sample preparation: Amnion derived adherent cells were obtained as described in Example 1. The cells at passage 6 were grown to approximately 70% confluence in growth medium as described in Example 1, above, trypsinized, and washed in PBS. NTERA-2 cells (American Type Culture Collection, ATCC Number CRL-1973) were grown in DMEM containing 4.5 g/L glucose, 2 mM glutamine and 10% FBS. Nucleated cell counts were performed to obtain a minimum of 2×10⁶ to 1×10⁷ cells. The cells were then lysed using a Qiagen RNeasy kit (Qiagen, Valencia, Calif.), utilizing a QIAshredder, to obtain the lysates. The RNA isolation was then performed using a Qiagen RNeasy kit. RNA quantity and quality were determined using a Nanodrop ND1000 spectrophotometer, 25 ng/μL of RNA/reaction. The cDNA reactions were prepared using an Applied Biosystems (Foster City, Calif.) High Capacity cDNA Archive Kit. Real time PCR reactions were performed using TAQMAN® universal PCR master mixes from Applied Biosystems. Reactions were run in standard mode on an Applied Biosystems 7300 Real time PCR system for 40 cycles.

Sample analysis and results: Using the real time PCR methodology and specific TAQMAN® gene expression probes and/or the TAQMAN® human angiogenesis array (Applied Biosystems), cells were characterized for expression of stem cell-related, angiogenic and cardiomyogenic markers. Results were expressed either as the relative expression of a gene of interest in comparison to the pertinent cell controls, or the relative expression (delta Ct) of the gene of interest in comparison to a ubiquitously expressed housekeeping gene (for example, GAPDH, 18S, or GUSB).

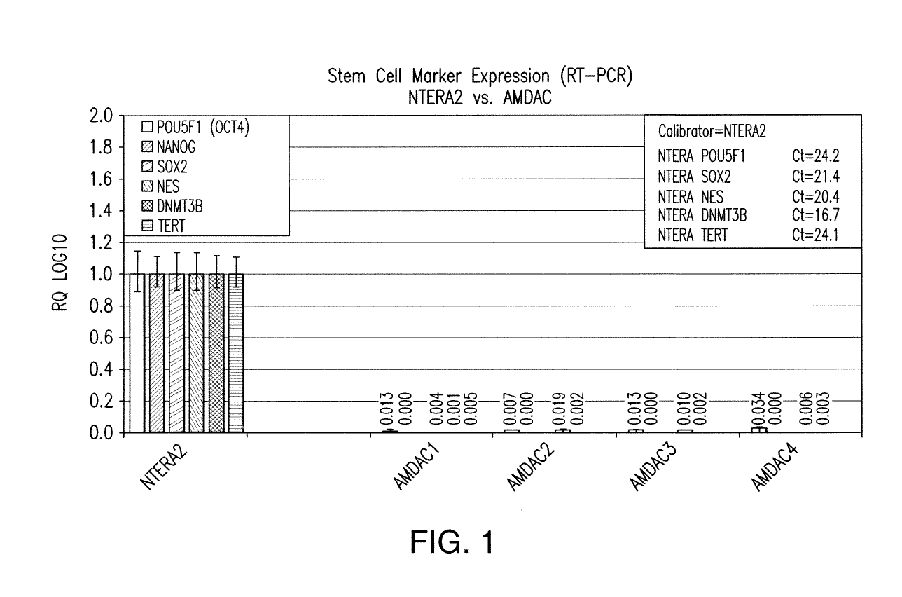

Amnion derived adherent cells expressed various, stem-cell related, angiogenic and cardiomyogenic genes and displayed a relative absence of OCT-4 expression in comparison to NTERA-2 cells. Table 8 summarizes the expression of selected angiogenic, cardiomyogenic, and stem cell genes, and FIG. 1 demonstrates the lack of expression in AMDACs of the stem cell-related genes POU5F1 (OCT-4), NANOG, SOX2, NES, DNMT3B, and TERT.

TABLE 8
Gene expression profile of amnion derived adherent cells
as determined by RT-PCR.

| AMDAC Marker | Positive | Negative |
|---|---|---|
| ACTA2 | X | |
| ACTC1 | X | |
| ADAMTS1 | X | |
| AMOT | X | |
| ANG | X | |
| ANGPT1 | X | |
| ANGPT2 | X | |
| ANGPT4 | | X |
| ANGPTL1 | X | |
| ANGPTL2 | X | |
| ANGPTL3 | | X |
| ANGPTL4 | X | |
| BAI1 | X | |
| BGLAP | | X |
| c-myc | X | |
| CD31 | | X |
| CD34 | | X |
| CD44 | X | |
| CD140a | X | |
| CD140b | X | |
| CD200 | X | |
| CD202b | X | |
| CD304 | X | |
| CD309 | X | |
| (VEGFR2/KDR) | | |
| CDH5 | | X |
| CEACAM1 | X | |
| CHGA | X | |
| COL15A1 | X | |
| COL18A1 | X | |
| COL4A1 | X | |
| COL4A2 | X | |
| COL4A3 | X | |
| Connexin-43 | X | |
| CSF3 | X | |
| CTGF | X | |
| CXCL10 | | X |
| CXCL12 | X | |
| CXCL2 | X | |
| DLX5 | | X |
| DNMT3B | X | |
| ECGF1 | X | |
| EDG1 | X | |
| EDIL3 | X | |
| ENPP2 | X | |
| EPHB2 | X | |
| F2 | X | |
| FBLN5 | X | |
| FGA | | X |
| FGF1 | X | |
| FGF2 | X | |
| FGF4 | | X |
| FIGF | X | |
| FLT3 | | X |
| FLT4 | X | |
| FN1 | X | |
| FOXC2 | X | |
| Follistatin | X | |
| Galectin-1 | X | |
| GRN | X | |
| HEY1 | X | |
| HGF | X | |
| HLA-G | | X |
| HSPG2 | X | |
| IFNB1 | X | |
| IFNG | | X |
| IL-8 | X | |
| IL-12A | X | |
| ITGA4 | X | |
| ITGAV | X | |
| ITGB3 | X | |
| KLF-4 | X | |
| LECT1 | | X |
| LEP | | X |
| MDK | X | |
| MMP-13 | | X |
| MMP-2 | X | |
| MYOZ2 | X | |
| NANOG | | X |
| NESTIN | | X |
| NRP2 | X | |
| PDGFB | X | |
| PF4 | X | |
| PGK1 | X | |
| PLG | | X |
| POU5F1 (OCT-4) | | X |
| PRL | | X |
| PROK1 | | X |
| PROX1 | X | |
| PTN | X | |
| SEMA3F | X | |
| SERPINB5 | X | |
| SERPINC1 | X | |
| SERPINF1 | X | |
| SOX2 | | X |
| TERT | | X |
| TGFA | X | |
| TGFB1 | X | |
| THBS1 | X | |
| THBS2 | X | |
| TIE1 | X | |
| TIMP2 | X | |
| TIMP3 | X | |
| TNF | X | |
| TNFSF15 | X | |
| TNMD | | X |
| TNNC1 | X | |
| TNNT2 | X | |
| VASH1 | X | |
| VEGF | X | |
| VEGFB | X | |
| VEGFC | X | |
| VEGFR1/FLT-1 | X | |
| XLKD1 | | X |

In a separate experiment, AMDACs were additionally found to express genes for Aryl hydrocarbon receptor nuclear translocator 2 (ARNT2), nerve growth factor (NGF), brain-derived neurotrophic factor (BDNF), glial-derived neurotrophic factor (GDNF), neurotrophin 3 (NT-3), NT-5, hypoxia-Inducible Factor 1α (HIF1A), hypoxia-inducible protein 2 (HIG2), heme oxygenase (decycling) 1 (HMOX1), Extracellular superoxide dismutase [Cu—Zn] (SOD3), catalase (CAT), transforming growth factor β1 (TGFB1), transforming growth factor β1 receptor (TGFB1R), and hepatoycte growth factor receptor (HGFR/c-met).

6.2.2 Flow Cytometry for Evaluation of Amnion Derived Adherent Cells

Flow cytometry was used as a method to quantify phenotypic markers of amnion derived adherent cells to define the identity of the cells. Cell samples were obtained from frozen stocks. Prior to thaw and during reagent preparation, cell vials were maintained on dry ice. Subsequently, samples were thawed rapidly using a 37° C. water bath. Pre-freeze cell counts were used for calculations for initial post-thaw cell number-dependent dilutions. Briefly, cryovials were thawed in a 37° C. water bath for approximately 30 seconds with gentle agitation. Immediately following thawing, approximately 100-200 μL of cold (2 to 8° C.) thawing solution (PBS with 2.5% albumin and 5% Gentran 40) was added to the cryovial and mixed. After gentle mixing, the total volume in the cryovials was transferred into a 15 mL conical tube containing an equal volume of cold (2 to 8° C.) thawing solution. The cells were centrifuged in a conical tube at 400 g for 5 minutes at room temperature before removing the supernatant. The residual volume was measured with a pipette (estimation); the residual volume and cell pellet were resuspended at room temperature in 1% FBS in PBS to achieve a cell concentration of 250×10³ cells/100 μL buffer. For example, 1×10⁶ cells would be resuspended in 400 μL 1% FBS. The cell suspension was placed into pre-labeled 5 mL FACS tubes (Becton Dickinson (BD), Franklin Lakes, N.J.). For each primary antibody isotype, 100 μL of cell suspension was aliquoted into one isotype control tube. Prior to phenotype analysis, the concentrations of all antibodies were optimized to achieve good signal to noise ratios and adequate detection of CD antigens across a potential four-log dynamic range. The volume of each isotype and sample antibody that was used to stain each sample was determined. To standardize the amount of antibody (in μg) in the isotype and sample tubes, the concentration of each antibody was calculated as (1/actual antibody concentration (μg/μL))×(desired final quantity of antibody in μg for 2.5×10⁵ cells)=# μL of antibody added. A master mix of antibodies for both the isotype and the sample was made with the appropriate amount of antibody added to each tube. The cells were stained for 15-20 minutes at room temperature in the dark. After staining, unbound antibody in each sample was removed by centrifugation (400 g×5 minutes) followed by washing using 2 mL 1% FBS PBS (room temperature) before resuspension in 150 μL of room temperature 1% FBS PBS. The samples were then analyzed on Becton Dickinson FACSCalibur, FACSCantoI or BD FACSCantoII flow cytometers prepared for use per manufacturer's instructions. Multi-parametric flow cytometry data sets (side scatter (SSC), forward scatter (FSC) and integrated fluorescence profiles (FL)) were acquired without setting on-the-fly instrument compensation parameters. Compensation parameters were determined after acquisition using the FACSDiva software according to the manufacturer's instructions. These instrument settings were applied to each sample. Fluorophore conjugates used in these studies were Allophycocyanin (APC), AlexaFluor 647 (AF647), Fluorescein isothiocyanate (FITC), Phycoerythrin (PE) and Peridinin chlorophyll protein (PerCP), all from BD Biosciences. Table 9 summarizes the expression of selected cell-surface markers, including angiogenic markers.

TABLE 9
Cell surface marker expression in amnion derived adherent cells
as determined by flow cytometry.

| AMDAC Marker | Positive | Negative |
|---|---|---|
| CD6 | | X |
| CD9 | X | |
| CD10 | X | |
| CD31 | | X |
| CD34 | | X |
| CD44 | X | |
| CD45 | | X |
| CD49b | X | |
| CD49c | X | |
| CD49d | X | |
| CD54 | X | |
| CD68 | X | |
| CD90 | X | |
| CD98 | X | |
| CD105 | X | |
| CD117 | | X |
| CD133 | | X |
| CD143 | | X |
| CD144 | | X |
| (VE-cadherin) | | |
| CD146 | | X |
| CD166 | X | |
| CD184 | | X |
| CD200 | X | |
| CD202b | X | |
| CD271 | | X |
| CD304 | X | |
| CD309 | X | |
| (VEGFR2/KDR) | | |
| CD318 | X | |
| CD349 | X | |
| CytoK | X | |
| HLA-ABC+ B2 | X | |
| Micro+ | | |
| Invariant Chain+ | | X |
| HLA-DR-DP-DQ+ | | |
| PDL-1 | X | |
| VEGFR1/FLT-1 | X | |

In another experiment, AMDAC cells were labeled with anti-human CD49f (Clone GoH3, phycoerythrin-conjugated; BD Pharmingen Part No. 555736), and analyzed by flow cytometry. Approximately 96% of the AMDACs labeled with anti-CD49f (that is, were CD49f).

In other experiments, AMDACs were additionally found by immunolocalization to express CD49a, CD106, CD119, CD130, c-met (hepatocyte growth factor receptor; HGFR), CXC chemokine receptor 1 (CXCR1), PDGFRA, and PDGFRB by immunolocalization. AMDACs were also found, by immunolocalization, to lack expression of CD49e, CD62E, fibroblast growth factor receptor 3 (FGFR3), tumor necrosis factor receptor superfamily member 12A (TNFRSF12A), insulin-like growth factor 1 receptor (IGF-1R), CXCR2, CXCR3, CXCR4, CXCR6, chemokine receptor 1 (CCR1), CCR2, CCR3, CCR4, CCR5, CCR6, CCR7, CCR8, CCR9, epidermal growth factor receptor (EGF-R), insulin receptor (CD220), interleukin receptor 4 (IL4-R; CD124), IL6-R (CD126), TNF-R1a and 1b (CD120a, b), and erbB2/Her2.

6.2.3 Immunohistochemistry (IHC)/Immunofluorochemistry (IFC) for Evaluation of Angiogenic Potency of Amnion Derived Adherent Cells

Amnion derived adherent cells from passage 6 were grown to approximately 70% confluence on 4-well chamber slides and fixed with a 4% formalin solution for 30 minutes each. After fixation, the slides were rinsed with PBS two times for 5 minutes. The slides were then incubated with 10% normal serum from the same host as the secondary antibody, 2× casein, and 0.3% Triton X100 in PBS, for 20 minutes at room temperature in a humid chamber. Excess serum was blotted off and the slides were incubated with primary antibody (goat polyclonal IgG (Santa Cruz; Santa Cruz, Calif.) in a humidified chamber. Time and temperature for incubations were determined by selecting the optimal conditions for the antibody being used. In general, incubation times were 1 to 2 hours at 37° C. or overnight at 4° C. The slides were then rinsed with PBS three times for 5 minutes each and incubated for 20-30 minutes at room temperature in a humid chamber with fluorescent-conjugated anti-immunoglobulin secondary antibody directed against the host of the primary antibody (rabbit anti-goat antibody (Santa Cruz)). Thereafter, the slides were rinsed with PBS three times for minutes each, mounted with a coverslip utilizing DAPI VECTASHIELD® (Vector Labs) mounting solution to counterstain nuclei. Cell staining was visualized utilizing a Nikon fluorescence microscope. All pictures were taken at equal exposure time normalized against the background of the corresponding isotype (goat IgG (Santa Cruz)). Table 10 summarizes the results for the expression of angiogenic proteins by amnion derived adherent cells.

TABLE 10
Angiogenic markers present or absent on amnion derived adherent cells.

| AMDAC | | |
|---|---|---|
| Marker | Positive | Negative |
| CD31 | | X |
| CD34 | | X |
| (VEGFR2/KDR) | X | |
| Connexin-43 | X | |
| Galectin-1 | X | |
| TEM-7 | X | |

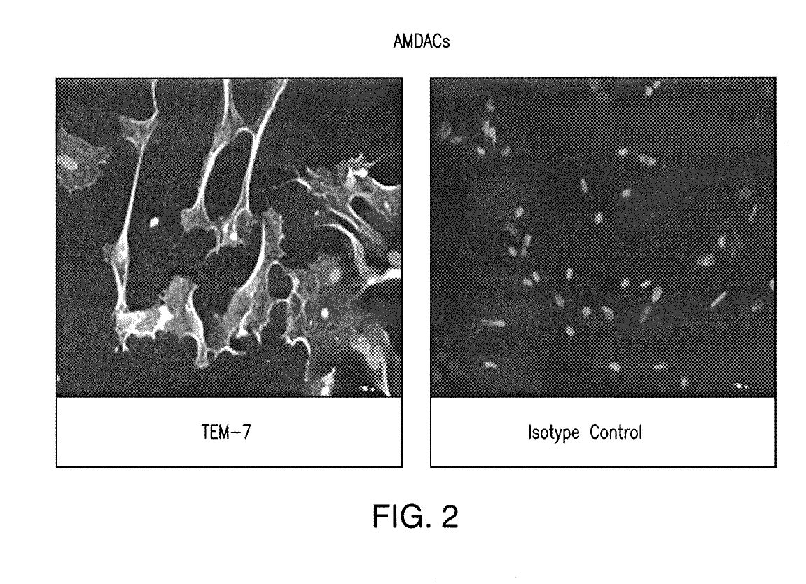

Amnion derived adherent cells expressed the marker tumor endothelial marker 7 (TEM-7), one of the proteins shown in Table 10. See FIG. 2.

6.2.4 Membrane Proteomics for Evaluation of Angiogenic Potency of Amnion Derived Adherent Cells

Membrane Protein Purification: Cells at passage 6 were grown to approximately 70% confluence in growth medium, trypsinized, and washed in PBS. The cells were then incubated for 15 minutes with a solution containing protease inhibitor cocktail (P8340, Sigma Aldrich. St. Louis, Mo.) prior to cell lysis. The cells were then lysed by the addition of a 10 mM HCl solution (thus avoiding the use of detergents) and centrifuged for 10 minutes at 400 g to pellet and remove the nuclei. The post-nuclear supernatant was transferred to an ultracentrifugation tube and centrifuged using a WX80 ultracentrifuge with a T-1270 rotor (Thermo Fisher Scientific, Asheville, N.C.) at 100,000 g for 150 minutes generating a membrane protein pellet.

Generation, Immobilization and Digestion of Proteoliposomes: The membrane protein pellet was washed several times using Nanoxis buffer (10 mM Tris, 300 mM NaCl, pH 8). The membrane protein pellet was suspended in 1.5 mL of Nanoxis buffer and then tip-sonicated using a VIBRA-CELL™ VC505 ultrasonic processor (Sonics & Materials, Inc., Newtown, Conn.) for 20 minutes on ice. The size of the proteoliposomes was determined by staining with FM1-43 dye (Invitrogen, Carlsbad, Calif.) and visualization with fluorescence microscopy. The protein concentration of the proteoliposome suspension was determined by a BCA assay (Thermo Scientific). The proteoliposomes were then injected onto an LPI™ Flow Cell (Nanoxis A B, Gothenburg, Sweden) using a standard pipette tip and allowed to immobilize for 1 hour. After immobilization, a series of washing steps were carried out and trypsin at 5 μg/mL (Princeton Separations, Adelphi, N.J.) was injected directly onto the LPI™ Flow Cell. The chip was incubated overnight at 37° C. and the tryptic peptides were eluted from the LPI™ chip and then desalted using a Sep-Pak cartridge (Waters Corporation, Milford, Mass.).

LTQ Linear Ion Trap LC/MS/MS Analysis: Each tryptic digest sample was separated on a 0.2 mm×150 mm 3 μm 200 Å MA GIC C18 column (Michrom Bioresources, Inc., Auburn, Calif.) that was interfaced directly to an axial desolvation vacuum-assisted nanocapillary electrospray ionization (ADVANCE) source (Michrom Bioresources, Inc.) using a 180 minute gradient (Buffer A: Water, 0.1% Formic Acid; Buffer B: Acetonitrile, 0.1% Formic Acid). The ADVANCE source achieves a sensitivity that is comparable to traditional nanoESI while operating at a considerably higher flow rate of 3 μL/min. Eluted peptides were analyzed on an LTQ linear ion trap mass spectrometer (Thermo Fisher Scientific, San Jose, Calif.) that employed ten data-dependent MS/MS scans following each full scan mass spectrum. Seven analytical replicate datasets were collected for each biological sample.

Bioinformatics: Seven RAW files corresponding to the 7 analytical replicate datasets that were collected for each cell line were searched as a single search against the IPI Human Database using an implementation of the SEQUEST algorithm on a Sorcerer Solo™ workstation (Sage-N Research. San Jose, Calif.). A peptide mass tolerance of 1.2 amu was specified, oxidation of methionine was specified as a differential modification, and carbamidomethylation was specified as a static modification. Scaffold software implementation of the Trans-Proteomic Pipeline (TPP) was used to sort and parse the membrane proteomic data. Proteins were considered for analysis if they were identified with a peptide probability of 95%, protein probability of 95% and 1 unique peptide. Comparisons between membrane proteomic datasets were made using custom Perl scripts developed in-house.

Results: As shown in Table 11, amnion derived adherent cells expressed various angiogenic and cardiomyogenic markers.

TABLE 11
Cardiomyogenic or angiogenic markers expressed
by amnion derived adherent cells.

| AMDAC Marker | Positive | Negative |
|---|---|---|
| Activin receptor | X | |
| type IIB | | |
| ADAM 17 | X | |
| Alpha-actinin 1 | X | |
| Angiotensinogen | X | |
| Filamin A | X | |
| Macrophage | X | |
| acetylated LDL | | |
| receptor I and II | | |
| Megalin | X | |
| Myosin heavy | X | |
| chain non | | |
| muscle type A | | |
| Myosin-binding | X | |
| protein C cardiac | | |
| type | | |
| Wnt-9 | X | |

6.2.5 Secretome Profiling for Evaluation of Angiogenic Potency of Amnion Derived Adherent Cells

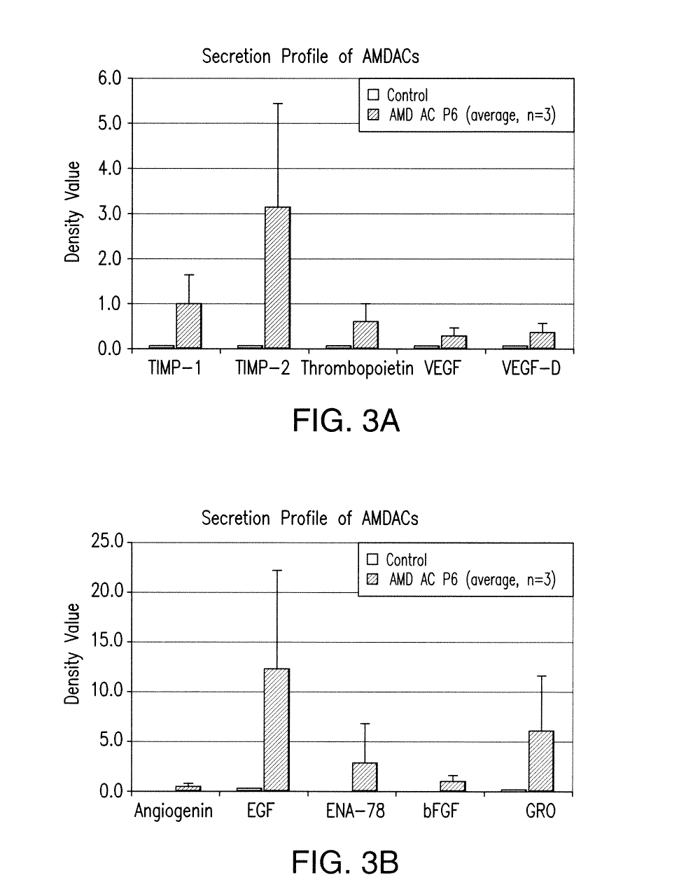

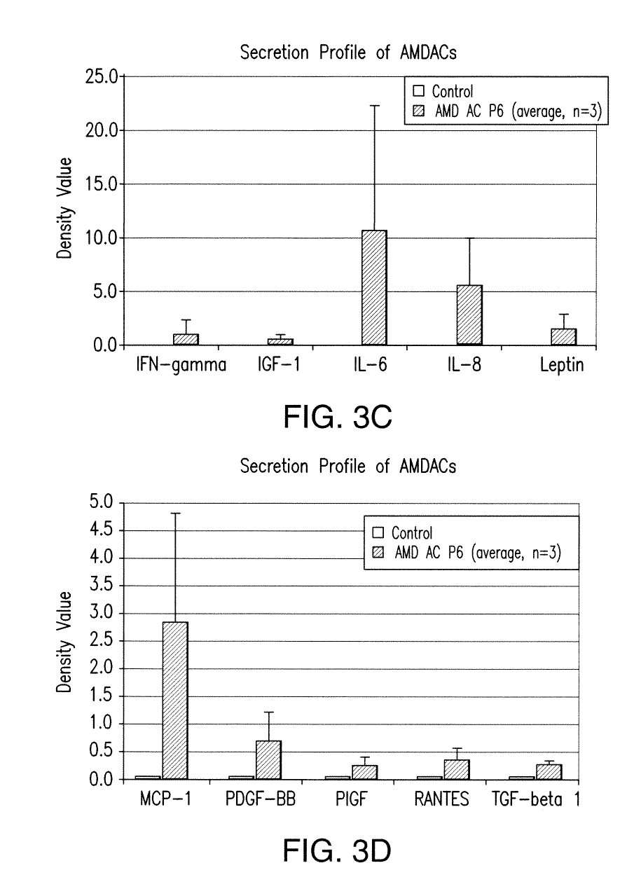

Protein Arrays: Amnion derived adherent cells at passage 6 were plated at equal cell numbers in growth medium and conditioned media were collected after 4 days. Simultaneous qualitative analysis of multiple angiogenic cytokines, growth factors in cell-conditioned media was performed using RayBiotech Angiogenesis Protein Arrays (Norcross, Ga.). In brief, protein arrays were incubated with 2 mL 1× Blocking Buffer (Ray Biotech) at room temperature for 30 minutes (min) to block membranes. Subsequently, the Blocking Buffer was decanted and the membranes were incubated with 1 mL of sample (growth medium conditioned by the respective cells for 4 days) at room temperature for 1 to 2 hours. The samples were then decanted and the membranes were washed 3×5 min with 2 mL of 1× Wash Buffer I (Ray Biotech) at room temperature with shaking. Then, the membranes were washed 2×5 min with 2 mL of 1× Wash Buffer II (Ray Biotech) at room temperature with shaking. Thereafter, 1 mL of diluted biotin-conjugated antibodies (Ray Biotech) was added to each membrane and incubated at room temperature for 1-2 hours and washed with the Wash Buffers as described above. Diluted HRP-conjugated streptavidin (2 mL) was then added to each membrane and the membranes were incubated at room temperature for 2 hours. Finally, the membranes were washed again, incubated with the ECL™ detection kit (Amersham) according to specifications and the results were visualized and analyzed using the Kodak Gel Logic 2200 Imaging System. The secretion of various angiogenic proteins by AMDACs is shown in FIG. 3.

ELISAs: Quantitative analysis of single angiogenic cytokines/growth factors in cell-conditioned media was performed using commercially available kits from R&D Systems (Minneapolis, Minn.). In brief, ELISA assays were performed according to manufacturer's instructions and the amount of the respective angiogenic growth factors in the conditioned media was normalized to 1×10⁶ cells. Amnion derived adherent cells (n=6) exhibited approximately 4500 pg VEGF per million cells and approximately 17,200 pg IL-8 per million cells.

TABLE 12
ELISA results for angiogenic markers

| AMDAC Marker | Positive | Negative |
|---|---|---|
| ANG | X | |
| EGF | X | |
| ENA-78 | X | |
| FGF2 | X | |
| Follistatin | X | |
| G-CSF | X | |
| GRO | X | |
| HGF | X | |
| IL-6 | X | |
| IL-8 | X | |
| Leptin | X | |
| MCP-1 | X | |
| MCP-3 | X | |
| PDGFB | X | |
| PLGF | X | |
| Rantes | X | |
| TGFB1 | X | |
| Thrombopoietin | X | |
| TIMP1 | X | |
| TIMP2 | X | |
| uPAR | X | |
| VEGF | X | |
| VEGFD | X | |

In a separate experiment, AMDACs were confirmed to also secrete angiopoietin-1, angiopoietin-2, PECAM-1 (CD31; platelet endothelial cell adhesion molecule), laminin and fibronectin. Other experiments confirmed that the AMDACs additionally secreted matrix metalloprotein (MMP) 1, MMP7, MMP9 and MMP10.

6.2.6 AMDAC MicroRNA Expression

This Example demonstrates that AMDACs express higher levels of certain micro-RNAs (miRNAs), and lower levels of certain other miRNAs, each of which correlated with angiogenic function, than bone marrow-derived mesenchymal stem cells.

It is known that pro-angiogenic miR-296 regulates angiogenic function through regulating levels of growth factor receptors. For example, miR-296 in endothelial cells contributes significantly to angiogenesis by directly targeting the hepatocyte growth factor-regulated tyrosine kinase substrate (HGS) mRNA, leading to decreased levels of HGS and thereby reducing HGS-mediated degradation of the growth factor receptors VEGFR2 and PDGFRb. See Würdinger et al., Cancer Cell 14:382-393 (2008). In addition, miR-15b and miR-16 have been shown to control the expression of VEGF, a key pro-angiogenic factor involved in angiogenesis, and that hypoxia-induced reduction of miR-15b and miR-16 contributes to an increase in VEGF, a pro-angiogenic cytokine. See Kuelbacher et al. Trends in Pharmacological Sciences, 29(I):12-15 (2007).

AMDACs were prepared as described in Example 1, above. AMDACs and BM-MSC cells (used as a comparator) were subjected to microRNA (miRNA) preparation using a MIRVANA™ miRNA Isolation Kit (Ambion, Cat#1560). 0.5×10⁶ to 1.5×10⁶ cells were disrupted in a denaturing lysis buffer. Next, samples were subjected to acid-phenol+chloroform extraction to isolate RNA highly enriched for small RNA species. 100% ethanol was added to bring the samples to 25% ethanol. When this lysate/ethanol mixture was passed through a glass fiber filter, large RNAs were immobilized, and small RNA species were collected in the filtrate. The ethanol concentration of the filtrate was then increased to 55%, and the mixture was passed through a second glass fiber filter where the small RNAs became immobilized. This RNA was washed, and eluted in a low ionic strength solution. The concentration and purity of the recovered small RNA was determined by measuring its absorbance at 260 and 280 nm.

AMDACs were found to express the following angiogenic miRNA: miR-17-3p, miR-18a, miR-18b, miR-19b, miR-92, miR-20a, miR-20b, (members of the of the angiogenic miRNA cluster 17-92), miR-296, miR-221, miR-222, miR-15b, miR-16. AMDACs were also found to express higher levels of the following angiogenic miRNA when compared to bone marrow-derived mesenchymal stem cells (BM-MSCs): miR-17-3p, miR-18a, miR-18b, miR-19b, miR-92 (members of the of the angiogenic miRNA cluster 17-92), miR-296. These results correlate well with the observation that AMDACs express high levels of VEGFR2/KDR (see above). Conversely, AMDACs were found to express lower levels of the following angiogenic miRNA when compared to BM-MSCs: miR-20a, miR-20b, (members of the of the angiogenic miRNA cluster 17-92), miR-221, miR-222, miR-15b, miR-16. The reduced expression of miR-15b and miR-16 correlated with the higher levels of expression of VEGF seen in AMDACs.

### 6.3 Example 3

### Differentiation of Amnion Derived Adherent Cells

### 6.3.1 Example 3.1

### Osteogenic Non-Differentiation of Amnion Derived Adherent Cells

This Example demonstrates that amnion derived adherent cells (AMDACs) do not differentiate into osteogenic cells, as established by, e.g., von Kossa staining, which stains for mineralization, e.g., calcium deposited by cells.

Cryopreserved OCT-4⁻ AMDACs, obtained as described in Example 1 above, were thawed, washed to remove dimethylsulfoxide (DMSO) and re-suspended in growth medium. The cells were seeded at 5000 cells/cm² in 24-well plates and 6-well plates in growth medium and incubated overnight. Subsequently, the medium was removed and replaced with osteogenic medium comprising DMEM-low glucose, 10% v/v fetal bovine serum (FBS), 10 mM beta glycerophosphate (Sigma), 100 nM dexamethasone (Sigma), 100 μM ascorbic acid phosphate salt (Sigma), fungizone (Gibco), 50 units/ml penicillin, and 50 μg/ml streptomycin (Gibco). The osteogenic medium was supplemented with 20 ng/ml transforming growth factor-beta1 (TGF-31) (Sigma), and 40 ng/ml human recombinant bone morphogenetic protein-2 (BMP-2) (Sigma). Culture of the AMDACs was continued in osteogenic medium for a total of 28 days with media changes every 3-4 days. At the end of the culture period, the cells were collected, washed, and stained as detailed below for evaluation of mineralization, an indicator or osteogenic differentiation. When observed under a microscope, the cell layer was fully confluent with fibroblastoid morphology (e.g., non-cuboidal in appearance), with no nodules observed.

As controls, dermal fibroblasts and bone marrow-derived mesenchymal stem cells (BM-MSCs) were cultured in the osteogenic medium as well. Adult normal human dermal fibroblasts (NHDF) were acquired from Lonza (Walkersville, Md., USA) and neonatal NHDF were acquired from ATCC (Manassas, Va., USA). Three BM-MSC lines from different origin were evaluated: one from ScienCell Laboratories (Carlsbad, Calif., USA), a second from Lonza (Walkersville, Md., USA), and a third was isolated from fresh whole normal bone marrow aspirates, obtained from AllCells (Emeryville, Calif., USA).

Cells were fixed with 10% (v/v) neutral buffered methanol. After fixation, the cells were washed in deionized water and incubated in 5% Silver Nitrate (Aldrich) for 1 hour under indirect UV light. The cells were then washed in deionized water and incubated in 5% (w/v) sodium thiosulphate for 5 minutes. The cells were then washed again in distilled water and examined by light microscopy.

Differential expression levels of osteogenic differentiation-related genes bone sialoprotein (IBSP) and osteocalcin (BGLAP), before and after induction, were evaluated by RT-PCR. Specifically, the AMDACs were received at the end of the osteogenesis differentiation assay, then lysed using RLT lysis buffer (Qiagen). Cell lysates were stored at −80° C. AMDAC cell lysates were thawed, and RNA was isolated using an RNEasy kit (Qiagen) per manufacturer's instructions with DNAse treatment. RNA was then eluted with DEPC treated water, and the RNA quantity was determined using a Nanodrop ND1000 spectrophotometer. cDNA was made from the RNA using Applied Biosystems reverse transcription reagents. Real time PCR reactions were done using Taqman Universal PCR master mix from Applied Biosystems. Taqman gene expression assays used were Hs00173720 Bone Sialoprotein, Hs00609452 Osteocalcin, and GAPDH. Real time PCR reactions were run in an ABI 7300 system as shown below:

| Stage | Repetitions | Temperature | Time | Ramp Rate |
|---|---|---|---|---|
| 1 | 1 | 50.0° C. | 2:00 | 100 |
| 2 | 1 | 95.0° C. | 10:00 | 100 |
| 3 | 40 | 95.0° C. | 0:15 per | 100 |
| | | 60.0° C. | 1:00 per | 100 |

Interpretation of Threshold Cycle (Ct) values:

Average Ct 1-10 very high expression

Average Ct 10-20 high expression

Average Ct 20-30 medium level expression

Average Ct 30-35 low expression

Average Ct 35-40 very low expression

Expression values (Ct) of each gene were normalized to that of the housekeeping gene GAPDH. The normalized expression values (dCt) of each Sample were then compared pre- and post-induction. The relative differences, in terms of fold-change, were reported as “RQ”. Due to the typical variability in dCt of housekeeping genes, any induction fold difference of less than 3 was not considered to be significant.

Results: Von Kossa staining results demonstrated that AMDACs were clearly nonosteogenic, as no von Kossa staining was detected. Control fibroblasts showed minimal mineralization, while BM-MSC displayed various degrees of mineralization.

TABLE 13
von Kossa Staining Results

| | | von Kossa |
|---|---|---|
| Cell Type | Donor ID | Staining Intensity |
| AMDAC | 1 | − (Negative) |
| AMDAC | 2 | − (Negative) |
| Dermal Fibroblast | 3 | + (Borderline Positive) |
| adult normal | | |
| Dermal Fibroblast | 4 | − (Negative) |
| neonatal normal | | |
| Bone Marrow MSC | 5 | ++++ (Positive) |
| Bone Marrow MSC | 6 | ++ (Positive) |
| Bone Marrow MSC | 7 | + (Borderline Positive) |

With respect to gene expression, all cells tested displayed moderate basal expression of osteocalcin (Ct 27.5-30.9). AMDACs demonstrated a marginal (<2 fold) induction of osteocalcin expression that was not deemed to be significant when compared to the induction of osteocalcin expression observed for fibroblasts or BM-MSC. As such, the induction of osteocalcin expression by AMDACs was not indicative of osteogenic potential. In contrast, 2 out of 3 BM-MSC lines showed substantial up-regulation upon induction. Variation in BM-MSCs for induction of bone sialoprotein is possibly due to donor variation.

TABLE 14
Gene Expression Results

| | | | | | | | |
|---|---|---|---|---|---|---|---|
| | Donor | | BGLAP | | dCt St. | Fold | GAPDH |
| Cell Type | ID | Condition | Ct | Avg. dCt | Dev. | Induction | Ct |

BGLAP (Osteocalcin)

| | | | | | | | |
|---|---|---|---|---|---|---|---|
| AMDAC | 2 | Basal | 28.2 | 10.2 | 0.07 | 1.6 | 18.0 |
| | | Induced | 29.2 | 9.5 | 0.12 | | 19.7 |
| Fibroblast | 3 | Basal | 28.2 | 10.0 | 0.11 | 0.8 | 18.2 |
| | | Induced | 28.4 | 10.4 | 0.12 | | 18.0 |
| | 4 | Basal | 29.5 | 10.8 | 0.24 | 0.6 | 18.6 |
| | | Induced | 30.7 | 11.6 | 0.18 | | 19.1 |
| BM-MSC | 5 | Basal | 27.7 | 9.9 | 0.09 | 0.3 | 17.8 |
| | | Induced | 30.9 | 11.7 | 0.14 | | 19.1 |
| | 6 | Basal | 27.5 | 9.9 | 0.12 | 0.3 | 17.6 |
| | | Induced | 29.8 | 11.6 | 0.07 | | 18.3 |
| | 7 | Basal | 27.0 | 9.5 | 0.10 | 0.3 | 17.6 |
| | | Induced | 29.8 | 11.0 | 0.16 | | 18.7 |

IBSP (Bone Sialoprotein)

| | | | | | | | |
|---|---|---|---|---|---|---|---|
| AMDAC | 2 | Basal | >40 | 17.7 | 0.10 | 0.13 | 18.0 |
| | | Induced | 38.6 | 20.6 | 0.12 | | 19.7 |
| Fibroblast | 3 | Basal | 35.8 | ND | 0.11 | NA | 18.2 |
| | | Induced | 38.6 | 18.9 | 0.12 | | 18.0 |
| | 4 | Basal | >40 | ND | 0.24 | NA | 18.6 |
| | | Induced | 38.2 | 19.1 | 0.18 | | 19.1 |
| BM-MSC | 5 | Basal | 33.6 | 15.8 | 0.09 | 0.066 | 17.8 |
| | | Induced | 38.9 | 19.7 | 0.14 | | 19.1 |
| | 6 | Basal | 35.7 | 18.1 | 0.12 | 4405 | 17.6 |
| | | Induced | 24.2 | 6.0 | 0.07 | | 18.3 |
| | 7 | Basal | 32.5 | 15.0 | 0.10 | 1508 | 17.6 |
| | | Induced | 23.1 | 4.4 | 0.16 | | 18.7 |

ND—Not detected
NA—Not able to calculate because uninduced condition was not detected (that is, no Ct value was determinable)

Fold induction values of 3 or less presented in Table 14 are not considered to be significant because of variability of expression of housekeeping genes used as comparators. Thus, based on the above results, it was concluded that AMDACs do not exhibit osteogenic potential.

### 6.3.2 Example 3.2

### Chondrogenic Non-Differentiation of Amnion Derived Adherent Cells

This Example demonstrates that amnion derived adherent cells, as described herein, do not differentiate along a chondrogenic pathway.

OCT-4⁻ AMDACs as described elsewhere herein were used in a chondrogenesis assay, along with dermal fibroblasts and BM-MSCs as controls. For each test sample, 0.25×10⁶ cells were placed in a 15 mL conical tubes and centrifuged at 200×g for 5 minutes at room temperature to form a spherical pellet. Pellets were cultured either in chondrogenic induction medium (Lonza Chondrocyte Medium (Lonza PT-3003)) containing TGF beta-3 (10 ng/mL), recombinant human growth/differentiation factor-5 (rhGDF-5) (500 ng/mL), or a combination of TGF beta-3 (10 nanogram/milliliter), and rhGDF-5 (500 ng/mL)) or in growth medium (DMEM-low glucose (Gibco)+FBS (2% v/v) (Hyclone)+Penicillin and Streptomycin) for three weeks. During culture, full exchanges of media were performed twice a week.

At the end of the culture period, cell pellets were fixed in 10% formalin for 24 hours. All samples were then dehydrated through graded alcohols and were embedded in paraffin. Sections were cut to a thickness of 5 μm and then stained according to protocols as described below. The histological sections were examined using light microscopy.

Alcian Blue Staining: When used in a 3% acetic acid solution (pH 2.5), Alcian Blue stains both sulfated and carboxylated acid mucopolysaccharides and sulfated and/or carboxylated sialomucins. 1% Alcian Blue (Sigma-Aldrich #23655-1) in 3% Acetic Acid was used, followed by a 0.1% Nuclear fast red (Sigma-Aldrich #22911-3) counterstain. In brief, the sections were deparaffinized and hydrated through graded alcohols to distilled water, stained in Alcian Blue for 30 minutes, washed in running tap water for two minutes, rinsed in distilled water, then counterstained in nuclear fast red solution for 5 minutes, washed in running tap water for 1 minute, dehydrated through graded alcohols, cleared in xylene and finally mounted with resinous mounting medium.

Type II Collagen Staining: The presence of Type II Collagen in cell culture samples before and after chondrogenic differentiation conditions are evaluated by immunohistochemistry as outlined below. Collagen H production by the cells was assessed using antibody 5B2.5 (Abcam Cat. # ab3092), a mouse monoclonal highly specific to type II collagen and which displays no cross reaction with types I, III, IV, V, VI, IX, X, or XI collagens, and no cross-reaction with pepsin-digested type II collagen. The assay used goat anti-mouse AF 594 (Invitrogen IgG2a, Cat#A21135) as a secondary antibody. Cell pellets were fixed in 10% formalin for a minimum of 4 hours to overnight and were infiltrated in paraffin.

All cell samples were washed in PBS and exposed to protein blocking solution containing PBS, 4% goat serum and 0.3% Triton-100× for 30 minutes at room temperature. Primary antibodies diluted in blocking solution (1:50 and 1:100) were then applied overnight at 4° C. Next morning, samples were washed in PBS, and secondary antibodies (goat-anti-mouse AF594) diluted in blocking solution (1:500) were applied for 1 hr at room temperature. The cells were then washed in PBS and 600 nM DAPI solution was applied for 10 minutes at room temperature to visualize nuclei.

BM-MSCs and fibroblasts formed cell pellets in chondrogenic induction medium. Chondrocytes formed large cell pellet with no distinct cell populations apically or centrally. In contrast, AMDACs failed to form a cell pellet during the culture period. No staining results were obtained for AMDACs for either collagen II or Alcian Blue because AMDACs failed to form cell pellets. Therefore, it was concluded that AMDACs are non-chondrogenic.

### 6.3.3 Example 3.3

### Neural Differentiation of Amnion Derived Adherent Cells

This Example demonstrates that amnion derived adherent cells can be differentiated to cells with characteristics of neural cells. Neural differentiation of the AMDACs was compared to that of normal human neuroprogenitors (Lonza), dermal fibroblasts, neonatal normal (Donor 3), Bone Marrow MSC (Donors 5 and 6).

In a first short term neural differentiation procedure, AMDACs and the other cells were thawed and expanded in their respective growth media after seeding at about 5000/cm² until they were sub confluent. Cells were trypsinized and seeded at 6000 cells per well in tissue culture-coated plate. All cells were initially expanded for 4 days in DMEM/F12 medium (Invitrogen) containing 15% v/v FBS (Hyclone), with basic fibroblast growth factor (bFGF) at 20 ng/ml, epidermal growth factor (EGF) at 20 ng/ml (Peprotech) and Penicillin/Streptomycin (PenStrep, Invitrogen). After 4 days, the cells were rinsed in PBS (Invitrogen). The cells were then cultured in DMEM/F12 with 20% v/v FBS, PenStrep for about 24 hours. After 24 hours, the cells were rinsed with PBS (Invitrogen) and cultured in induction medium consisting of DMEM/F12, serum free, containing 200 mM butylated hydroxyanisole, 10 nM potassium chloride, 5 mgs/mL insulin, 10 nM forskolin, 4 nM valproic acid, and 2 nM hydrocortisone (Sigma). The cells were subsequently fixed at −20° C. with 100% methanol. Fixed samples were then evaluated by immunohistochemistry (IHC) for expression of human nestin using an anti-nestin antibody (Alexa-Fluor 594 (Red) conjugated), with counterstaining with DAPI for nuclei.

In a second short term neural differentiation protocol, all cells were initially expanded for 4 days in DMEM/F12 medium (Invitrogen) containing 15% FBS (Hyclone), with basic FGF at 20 ng/ml, EGF at 20 ng/ml and PenStrep (Invitrogen). After 4 days, the cells were rinsed in PBS (Invitrogen) and were cultured in DMEM/F12 with 20% v/v FBS, PenStrep. After 24 hrs, cells were rinsed with PBS. The media were then switched to Neural Progenitor Expansion medium (NPE), which comprised NEUROBASAL™-A basal medium (Gibco), with B27 (Gibco), 4 mM L-glutamine, 1 μM retinoic acid (Sigma), and PenStrep. After four days, the medium was removed from each well and cells were fixed with ice cold 4% w/v paraformaldehyde for 10 minutes at room temperature. Fixed samples were then evaluated by IHC for expression of GFAP (glial fibrillary acidic protein) for astrocyte phenotype, and TuJ1 (neuron-specific class III tubulin) for neuronal phenotype, respectively.

In the first differentiation protocol, all cell types transformed into a cell type with bipolar morphology and stained positive with nestin. Neuroprogenitors constitutively expressed nestin as expected. In the second differentiation protocol, expression of neuronal-related (Tuj1) and astrocyte-related (GFAP) markers were evaluated. Upon induction, AMDAC, and BM-MSC expressed low levels of Tuj1. Expression on fibroblasts was found to be borderline positive which could be due to background. AMDACs, and one BM-MSC cell line, exhibited low-level expression of GFAP. The positive control cell line (neuroprogenitors) constitutively expressed both Tuj1 and GFAP, as expected.

Thus, AMDACs are able, under neural inducing conditions, to exhibit morphological and biochemical changes consistent with neural differentiation.

### 6.4 Example 4

### Immunomodulation Using Amnion Derived Angiogenic Cells

This example demonstrates that AMDACs display immunosuppressive function in vitro in an assay utilizing bead-stimulated T cells.

6.4.1 AMDAC-Mediated Suppression of T Cell Proliferation

AMDACs were obtained as described in Example 1, above. CD4⁺ and CD8⁺ T cells were obtained from human peripheral blood.

The T cells were labeled with carboxyfluorescein succinimidyl ester (CFSE) and mixed with anti-CD3 anti-CD28-coated Dynabeads, followed by culture in the absence of the AMDACs or a coculture with the AMDACs in a manner that allowed cell to cell contact, also known as a Bead T-lymphocyte reaction (BTR). Coculture with the AMDACs was performed by mixing 100,000 T-lymphocytes with anti-CD3 and anti-CD28 coated DynaBeads (Invitrogen) at a bead:T-lymphocyte ratio of 1:3 in a well of a 96-well plate, in the presence or absence of 20,000 AMDAC cells. The mixed (coculture) and unmixed cell cultures were incubated at 37° C., 5% CO₂, and 90% relative humidity for 5 days. Normal human dermal fibroblasts (NHDF), which do not possess substantial T cell inhibitory activity were used as a negative control, and subjected to the same conditions as the AMDACs.

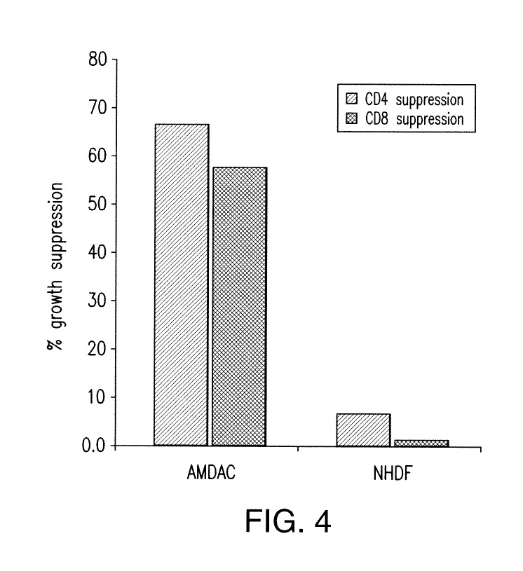

Following the 5 days, CFSE fluorescence on the CD4+ and CD8+ T cells was detected using flow cytometry, and the percentage of suppression of T cell growth was calculated based on the increased fraction of non-proliferated (CFSE high) T cells compared to the culture of CFSE-labeled T cells that were not co-cultured with AMDACs or NHDF. As demonstrated in FIG. 4, AMDACs inhibit the proliferation of CD4⁺ and CD8⁺ T cells in vitro, indicating that AMDACs are immunomodulatory.

6.4.2 Media Conditioned by AMDACs Inhibits Secretion of TNF-Alpha by T cells

AMDACs were obtained as described in Example 1, above. T cells were obtained from human peripheral blood.

The AMDACs were seeded on tissue culture plates and incubated overnight to form an adherent monolayer. The next day, the AMDAC culture was stimulated with IL-1 beta, which has previously been shown to be a potent inducer of AMDAC-derived anti-inflammatory factors. After 16 h of IL-1 beta stimulation, the medium conditioned by the AMDACs was collected and mixed at a 9:1 volume ratio with human peripheral blood T cells coated with anti-CD3 anti-CD28-coated Dynabeads. A separate population of human peripheral blood T cells coated with anti-CD3 anti-CD28-coated Dynabeads was maintained as a control. The T cells mixed with AMDAC-conditioned medium and the unmixed population of T cells were incubated at 37° C., 5% CO2, and 90% relative humidity for 72 h. Medium conditioned by normal human dermal fibroblasts (NHDF), which do not possess substantial TNF-alpha inhibitory activity was used as a negative control, and subjected to the same conditions as the AMDACs.

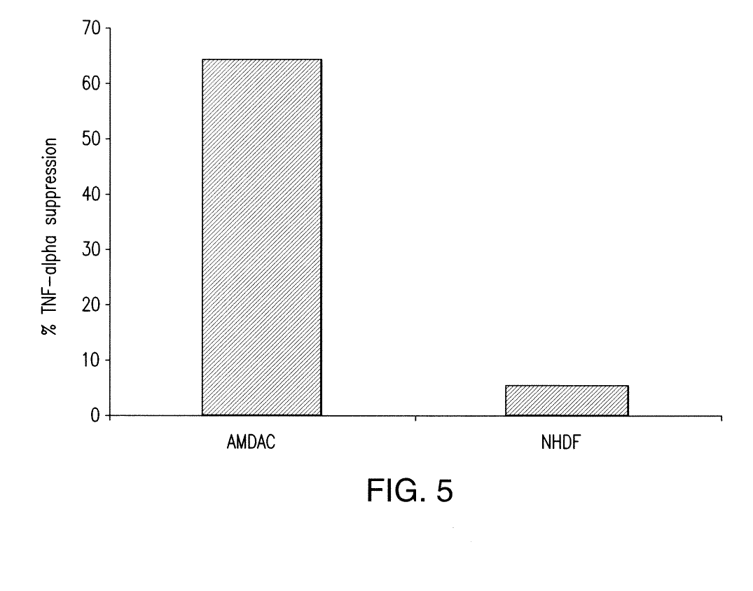

Following the 72 h culture, the concentration of T-cell derived TNF-alpha was measured in the T cell culture supernatants using a cytometric bead-based ELISA method. The percent suppression of TNF-alpha secretion was calculated based on the decrease of TNF-alpha concentration in the presence of AMDAC-conditioned medium compared to the control T cell culture which was not mixed with AMDAC-conditioned medium. As demonstrated in FIG. 5, the culture of the T cells in the presence of AMDAC-conditioned medium induced the suppression of production of T cell derived TNF-alpha.

### 6.5 Example 5

### AMDACS Modulate the T Cell Compartment

This Example demonstrates that amnion derived adherent cells (AMDACs), obtained as described in Example 1, are able to influence skewing in the Th1, Th17 and FoxP3 T_{reg} subsets.

6.5.1 Methods

T-Lymphocyte Proliferation Assays

Mixed lymphocyte reactions (MLR) were performed by mixing 100,000 HLA-mismatched carboxyfluorescein succinimidyl ester (CFSE)-labeled T-lymphocytes with 10,000 mature dendritic cells (mDC) in each well of a FALCON flat bottom 96 well tissue culture plate (Fisher Scientific, Pittsburg, Pa.) in the presence or absence of 20,000 AMDAC cells, isolated as described in Example 1, above. The mixed cell culture was incubated at 37° C., 5% CO₂, and 90% relative humidity for 6 days. At day 6 all cells were recovered and stained with anti-CD4-PE and anti-CD8-APC(R&D systems, Minneapolis, Minn.).

Bead T-lymphocyte reactions (BTR) were performed by mixing 100,000 T-lymphocytes with anti-CD3 and anti-CD28 coated DynaBeads (Invitrogen) at a bead:T-lymphocyte ratio of 1:3 in a well of a 96-well plate. The BTR reaction was performed in the presence or absence of 20,000 AMDAC cells. The mixed cell culture was incubated at 37° C., 5% CO₂, and 90% relative humidity for 6 days. At day 6 all cells were recovered and stained with anti-CD4-PE and anti-CD8 APC (R&D systems, Minneapolis, Minn.).

T-lymphocyte proliferation was measured by analysis of CFSE fluorescent intensity on CD4 and CD8 single positive cells with a FACS Canto II machine (BD, Franklin Lake, N.J.). All FACS data in this study were analyzed by using flowjo 8.7.1 software (Tree Star, InC. Ashland Oreg.).

T Cell Skewing (Polarization)

Th1 skewing was carried out using BTR reactions with an additional Th1 skewing cytokine cocktail containing IL-2 (200 IU/ml), IL-12 (2 ng/ml) and anti-IL-4 (0.4 μg/ml).

For Th17 skewing, 5×10⁵ total T-lymphocytes were stimulated with 5×10⁵ sorted CD14⁺ monocytes, 50 ng/mL anti-CD3 antibody (BD BioScienences) and 100 ng/mL LPS (Sigma Aldrich) in either the presence or absence of 50,000 AMDACs for 6 days. The Th17 cell population was analyzed by intracellular cytokine staining (ICCS) staining of IL-17 on the CD4 positive population.

Intracellular Cytokine and Foxp3 Staining

The Th1 cell subset was enumerated as follows. T cells from BTR reactions were re-activated with 50 ng/mL phorbol myristate acetate (PMA) and 750 ng/mL ionomycin (PI) (Sigma Aldrich) for 5 hours. GOLGISTOP™ (Becton Dickinson; a protein transport inhibitor) was added during the last 3 hours. Cells were then surface stained with PE labeled anti-CD4 antibody and subsequently with APC conjugated anti-IFN-γ antibody with the Cytofix/Cytoperm kit (Becton Dickinson) according to the manufacturer's instructions.

In order to enumerate the Th17 cell subset, T cells from a Th17 skewing activation reaction were re-activated with 50 ng/mL PMA and 750 ng/mL ionomycin (Sigma Aldrich) for 5 hours with GOLGISTOP™ (Becton Dickinson) present during the last 3 hours. Cells were then stained with PE labeled anti-CD4 antibody and subsequently with APC conjugated anti-IL-17 antibody with the Cytofix/Cytoperm kit (Becton Dickinson) according to the manufacturer's instructions.

In order to enumerate the Treg cell subset, T cells from BTR reactions were surface stained with PE labeled anti-CD4 antibody and subsequently with APC conjugated anti-Foxp3 antibody using the Foxp3 staining kit (eBioscience, San Diego, Calif.) according to the manufacturer's instructions.

Dendritic Cell Differentiation and Stimulation

Immature DC (iDC) were generated from a magnetically sorted CD14⁺ monocyte population by mitogen-directed differentiation. Briefly, iDCs were obtained from monocytes cultured at 1×10⁶/ml with GM-CSF (20 ng/ml) and IL-4 (40 ng/ml) for 4 days. iDCs (1×10⁵ cells) were then stimulated with 1 μg/mlLPS for 24 hours in either the absence or presence of 1×10⁵ AMDACs in each well of a FALCON 24 well tissue culture plate (Fisher Scientific, Pittsburgh, Pa.). Culture supernatant was collected and the cytokine profile was analyzed by Cytometric Bead Array (CBA).

Cytometric Bead Array (CBA) Analysis

Cytokine concentrations were measured in culture supernatants using the Cytometric Bead Array system (CBA; Becton Dickinson) for the simultaneous quantitative detection of multiple soluble analytes according to the manufacturer's instructions. Briefly, samples of BTR culture supernatants were incubated with a mix of capture beads for specific detection of the following cytokines produced by activated T cells: IL-2, IL-4, IL-5, IL-10, TNF, lymphotoxin-alpha (LT-α) and IFN-γ. Subsequently, bead bound cytokines were coupled with fluorescently labeled detection reagents and detected using the FACSCanto II flow cytometer following the manufacturer's protocols. Data was acquired and analyzed using the FACS-DIVA 6.0 software (Becton Dickinson), followed by calculation of cytokine concentrations using the FCAP Array 1.0 program (Becton Dickinson).

IL-21 ELISA

Soluble IL-21 was measured in supernatant obtained from Th17 skewing cultures with the IL-21 ELSAI kit from eBioscience (88-7216) according to the manufacturer's protocol.

NK Proliferation Assay and NK Cytotoxicity Assay

Human NK cells were isolated from PBMC using an NK cell isolation kit (Miltenyi Biotech, Auburn, Calif.) according to the manufacturer's instructions. NK cell proliferation was determined by culturing 2.5×10⁵ NK cells in 1 ml IMDM containing 10% fetal bovine sera (FBS) (Hyclone) supplemented with 35 μg/ml transferrin, 5 μg/ml insulin, 20 □M ethanolamine, 1 μg/ml oleic acid, 1 μg/ml linoleic acid, 0.2 μg/ml palmitic acid, 2.5 μg/ml BSA, 0.1 μg/ml PHA (Sigma-Aldrich) and 200 IU/ml human IL-2 (R&D), together with mitomycin C treated (16 g/ml) feeder cells (either 1×10⁶ human allogeneic PBMC or 1×10⁵ K562 cells). Cells were incubated at 37° C. in 5% CO₂ with the addition of an equal volume of IMDM (10% FBS, 2% human serum and 400 IU/ml IL-2) every 3 days. NK cell number was determined by FACS every seven days as follows. Briefly, total NK cells were collected from the tissue culture well. After washing with PBS, cells were then stained with 2 uM TO-PRO3. Finally, 10 μl counting beads (Spherotech, Cat# ACBP-50-10) were added to each sample which served as an internal standard for calibration of total cell number. Relative NK number was calculated based on the number of total live NK cells per 1000 counting beads collected.

The NK cytotoxicity assay was carried out by mixing NK cells with target cells at different effector/target (E/T) ratios. After overnight culture, target cell numbers were determined using the counting beads method described above plus cell surface markers to differentiate NK cells from target cells. For NK cytotoxicity of K562 cells, FITC conjugated anti-HLA-ABC antibodies were used as the NK cell marker, because K562 cells are HLA-ABC negative. For AMDAC cells, CD90-PE was used to distinguish AMDACs from NK and K562 cells. Percent cytotoxicity was calculated as (1−total target number in sample÷total target cells in a control containing no NK cells)×100.

6.5.2 AMDACs Skewing of T Cell Compartment

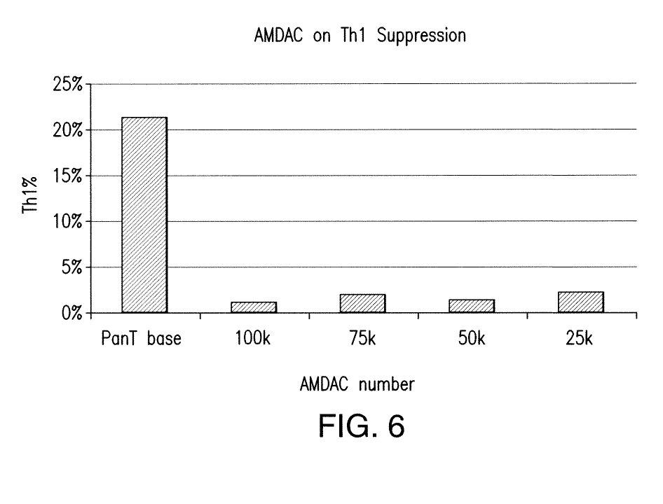

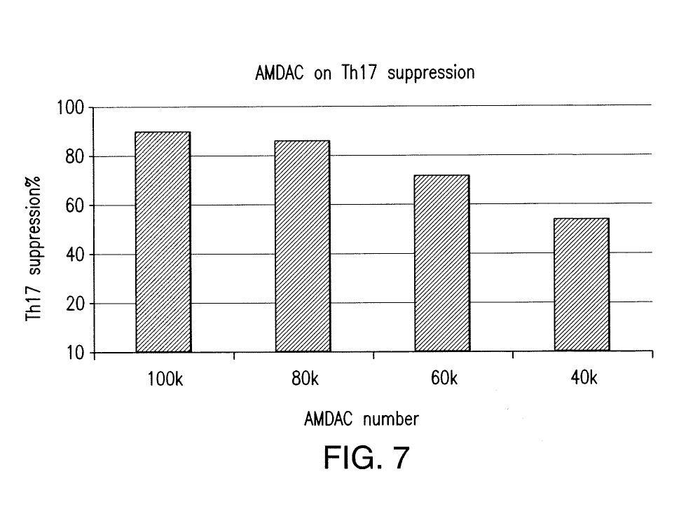

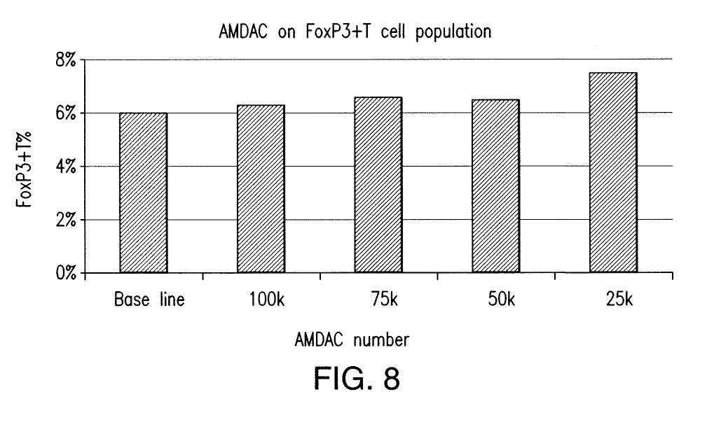

The ability of AMDACs to influence skewing in the T cell compartment was examined by measuring cytokine producing T cells in Th1 and Th17 skewing assays using T cell and AMDAC co-cultures. Briefly, in the Th1 skewing assay, AMDACs were pre-plated. The following day, 1×10⁶/ml T cells, Dynabeads at 6×10⁵/ml, IL-2 (200 IU/ml), IL-12 (2 ng/ml), and anti-IL-4 (0.4 μg/ml) were added and mixed with the AMDACs. Four days later, the percentage of Th1 cells was analyzed by interferon-gamma (IFN-γ) intracellular staining. As shown in FIG. 6, AMDACs greatly reduced the Th1 percentage in a dose dependent manner. Similarly, in a Th17 skewing assay, AMDACs were pre-plated overnight. A mixture of T cells (1×10⁶/ml), CD14⁺ cells (1×10⁶/ml), anti-CD3 (50 ng/ml) and bacterial lipopolysaccharide (LPS) (100 ng/ml) was then added to the plate containing AMDACs. After a six day culture, the Th17 percentage was examined by IL-17 intracellular staining. As shown in FIG. 7, AMDACs suppressed the Th17 percentage in a dose dependent manner. To investigate the effect of AMDACs on a FoxP3 positive T cell population, 1×10⁶ PBMC were co-cultured with AMDACs for 6 days. The FoxP3 positive population was analyzed by FoxP3 intracellular staining. As shown in FIG. 8, AMDACs slightly increased the FoxP3 positive T cell population.

6.5.3 AMDAC Mediated Modulation of DC Maturation and Function

This experiment demonstrates that AMDACs modulate the maturation and differentiation of immature dendritic cells (DCs).

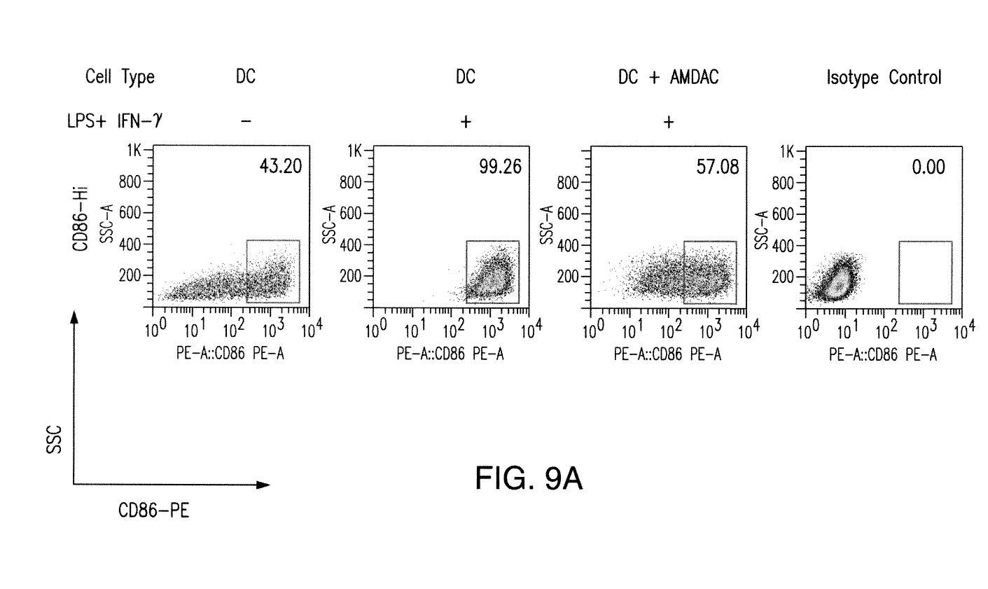

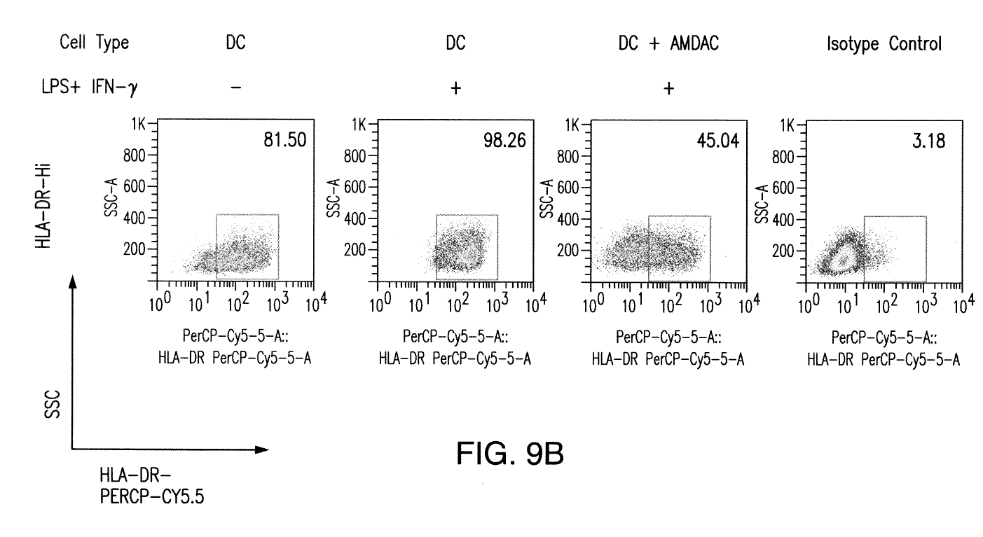

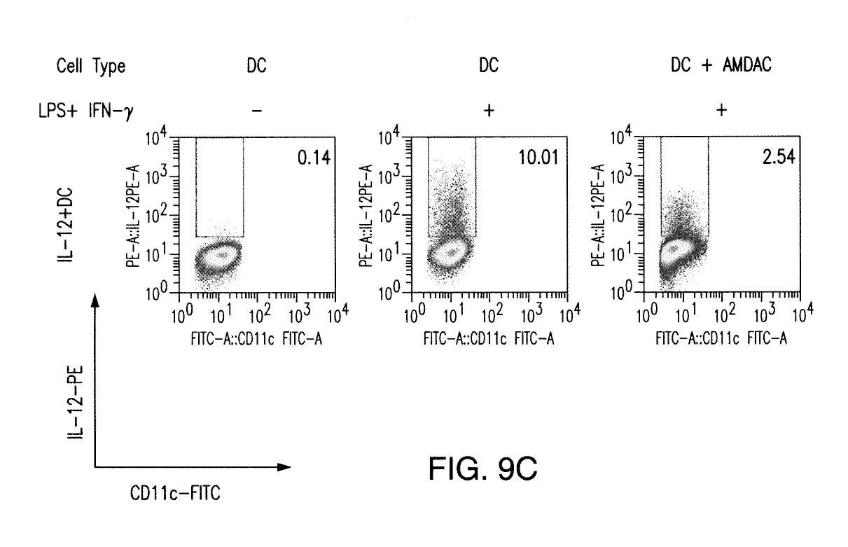

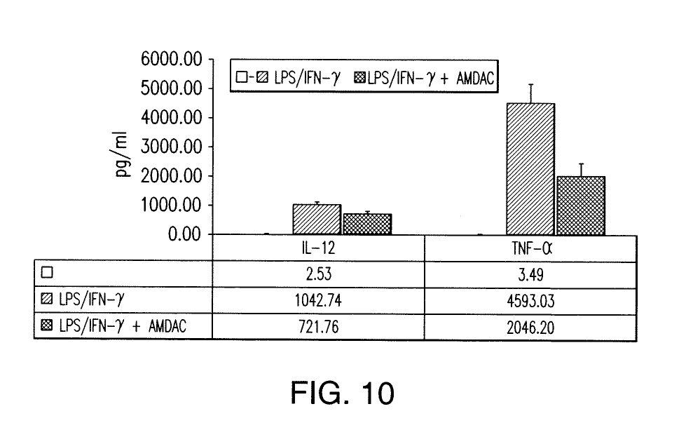

To explore the AMDAC mediated modulation of DC maturation and function, monocyte derived immature DCs were treated with LPS alone or a combination of LPS plus IFN-γ in the absence or presence of AMDACs to further drive the DC maturation process. DC maturation was analyzed by FACS staining of DC maturation markers CD86 and HLA-DR. DC function was assessed by intracellular staining of IL-12 and measurement of soluble cytokine production by CBA. As shown in FIGS. 9A and 9B, AMDACs strongly suppressed LPS and LPS plus IFN-γ-induced DC maturation by down-modulation of CD86 (FIG. 9A) and HLA-DR expression (FIG. 9B) on DCs. Further, as shown in FIG. 9C, AMDACs significantly suppressed the LPS plus IFN-γ-stimulated IL-12-producing DC population by ˜70%. AMDACs were further found to be able to suppress TNF-α and IL-12 production by LPS-stimulated DCs. See FIG. 10.

6.5.4 AMDACs Suppress IL-21 Production in a Th17 Skewing Culture

IL-21 is an important cytokine required for maintenance of a Th17 population. To investigate whether AMDACs are able to modulate IL-21 production, AMDACs were introduced into a Th17 skewing culture as described in the Methods section. AMDACs suppressed IL-21 production by 72.35% in AMDAC-Th17 co-cultures in comparison to a Th17 skewing culture without AMDAC cells. Additionally, AMDACs reduced the population of Th17 T cells by 72.65% as compared to culture in the absence of AMDACs.

6.5.5 AMDAC Modulation of NK Cell Cytotoxicity and Proliferation

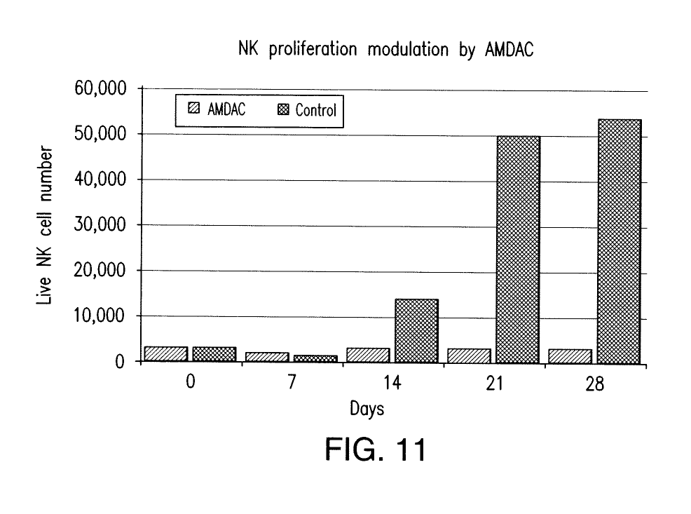

NK cells are a type of cytotoxic lymphocyte that constitutes a major component of the innate immune system. NK cells play a major role in the rejection of tumors and cells infected by viruses as well as allogeneic cells and tissues. To investigate the immunomodulatory effect of AMDACs on NK cells, NK cell proliferation and cytotoxicity assays were established. As shown in FIG. 11, AMDACs suppressed human NK cell proliferation in comparison to a control having no AMDAC cells.

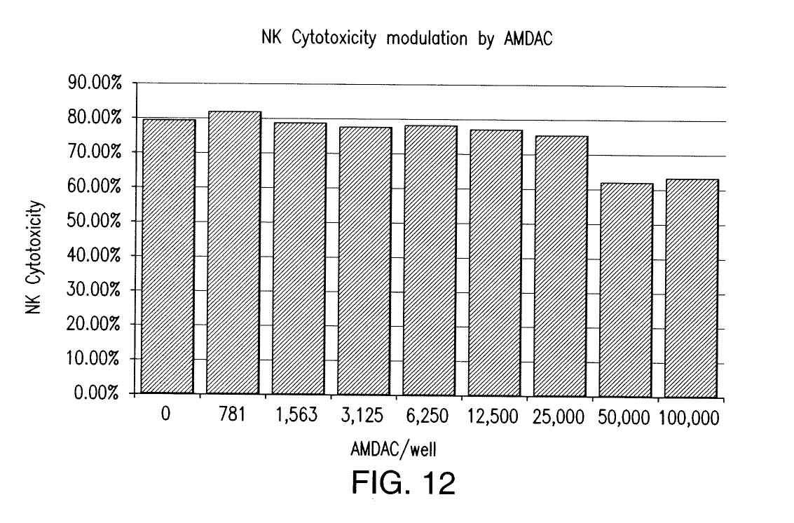

In addition, the effect of AMDACs on NK cell cytotoxicity was investigated. In this assay, AMDACs were introduced into an NK cytotoxicity assay as described in the Methods section above. Briefly, 1×10⁶ NK cells were mixed with 1×10⁵ K562 cells (E/T ratio of 10:1) with a 2 fold titration of pre-seeded AMDACs (1×10⁵ cells). The NK cells and K562 cells were co-cultured overnight, and NK cell cytotoxicity was determined according to the protocol described in the Methods section above. As shown in FIG. 12, AMDAC cells suppressed human NK cell cytotoxicity in a dose dependent manner.

### 6.6 Example 6

### In Vivo Model for Treating Critical Limb Ischemia with Compositions Comprising AMDACS and PRP

This example describes experiments that are performed in order to assess treatment of critical limb ischemia with compositions comprising AMDACs, as described herein, and platelet rich plasma (PRP).

Two rodent hind limb ischemia models are surgically induced, including: (1) a chronic mild ischemia model, is induced by cutting the femoral artery just below the bifurcation of the deep femoral artery; and (2) a stable severe ischemia model, which is induced by resection of the femoral artery from the distal site of the bifurcation of the deep femoral artery to the saphenous artery. Each group is subsequently treated with AMDACs only, PRP only, and AMDACs in combination with PRP. The amounts of AMDACs, and the ratio of AMDACs to PRP, are varied to assess dose-dependency of the different treatments.

Blood flow, in particular, calf blood flows on both sides are measured below a patella with a noncontact laser Doppler flowmeter before the surgical induction of ischemia, just after the surgical induction, before administration of the compositions as described above, and two weeks post-administration, and are expressed as the ratio of the flow in the ischemic limb to that in the normal limb, for each treatment group. At two weeks post-administration, the animals are sacrificed under an overdose of sodium pentobarbital and the anterotibial, gastrocnemius, and soleus muscles are dissected out and weighed. Histological analysis (HE staining) is performed in each muscle.

### 6.7 Example 6

### In Vivo Models for Treating Bone Repair and Disc Degeneration with Compositions Comprising AMDACS and PRP

This example describes experiments that are performed in order to assess treatment of bone defects with compositions comprising AMDACs, as described herein, and platelet rich plasma (PRP). Several models of bone disease are adapted to assess the efficacy of such treatments on different bone diseases.

To model cranial bilateral defect, a defect of 3 mm×5 mm is surgically created on each side of the cranium of male athymic rats. The defects are treated with matrix only, AMDACs only, PRP only, matrix in combination with AMDACs, matrix in combination with PRP, and matrix in combination with AMDACs and PRP. The amounts of AMDACs, and the ratio of AMDACs to PRP, are varied to assess dose-dependency of the different treatments. Different matrix materials are also assessed in order to test the effects of different combinations of matrix, stem cells, and PRP.

Six rats are assigned to each treatment group and the defects are filled with the designated matrix and cell combination. At four weeks, serum is collected and rats are sacrificed. Serum is tested for immunologic reaction to the implants. Rat crania are collected for microradiography and placed in 10% NBF.

Calvariae are processed for paraffin embedding and sectioning. Coronal histological sections of the calvariae are stained with toluidine stain according to conventional techniques. Bone ingrowth into the defect and remnant of matrix carrier is assessed according to a 0 to 4 scale, with four being the largest amount of ingrowth. Inflammation and fibrosis is also assessed.

Treatment of bone lesions resulting from cancer metastases can be assessed as follows. Site-specific osteolytic lesions are induced in nude rats by intra-arterial injection of human breast cancer cells into an anastomosing vessel between the femoral and the iliac arteries. The metastases are then either treated with conventional anti-cancer therapies (e.g., chemotherapeutic, radiological, immunological, or other therapy) or surgically removed. Next, the lesions remaining from the cancer metastases are tilled with different matrix combinations as described above. After an appropriate period of time, as determined by radiologically monitoring the animals, the animals are sacrificed. Immunologic response against the matrix, inflammation, fibrosis, degree of bone ingrowth, and amount of matrix carrier are assessed.

Treatment of disc degeneration can be assessed as follows. Rats are subjected to sham exposure or disc puncture. In rats receiving sham exposure only, the left facet joint between the 4th and the 5th lumbar vertebra is removed and the 4th lumbar dorsal root ganglion and the 5th lumbar nerve root, including the intervertebral disc between the fourth and fifth lumbar vertebrae (L4 and L5, respectively), are visualized. In rats subjected to disc puncture, the L4-L5 intervertebral disc is further punctured using a 0.4-mm diameter injection needle. Leakage of the nucleus pulposus is facilitated by injecting a small amount of air into the center of the disc.

Rats subjected to sham exposure or punctured discs are treated with AMDACs only, PRP only, and AMDACs in combination with PRP. The amounts of AMDACs, and the ratio of AMDACs to PRP, are varied to assess dose-dependency of the different treatments. Six rats are assigned to each treatment group and the defects are filled with the designated matrix and cell combination. The spinal muscles are sutured and the skin is closed by metal-clips.

After surgery, each rat receives a unique identification number to allow for a blinded behavioral assessment. Behavioral Testing Behavioral analysis is performed on days 1, 3, 7, 14, and 21 after surgery. Previous reports indicate that rats subjected to disc puncture, when compared to rats receiving only sham exposure, display increased grooming behavior and “wet-dog shaking” (WDS), a behavior that resembles a wet dog that is shaking to remove water from the fur. These two behaviors are suggested to indicate stress and pain. Thus, the ability of AMDACs and PRP, alone or in combination, to suppress or ameliorate these behaviors in rats subjected to disc puncture are assessed.

### 6.8 Example 8

### In Vivo Model for Treating Neuropathic Pain with Compositions Comprising AMDACS and PRP

This Example provides an exemplary model and method for evaluating the effects of a composition comprising AMDACs and PRP in a rat model for chronic, painful peripheral mononeuropathy.

Peripheral mononeuropathy is surgically induced in rats as follows. Rats are anesthetized with sodium pentobarbital (40 mg/kg. i.p.). The common sciatic nerve is exposed at the level of the middle of the thigh by blunt dissection through biceps femoris. Proximal to the sciatic nerve's trifurcation, about 7 mm of nerve is freed of adhering tissue and 4 ligatures (4.0 chromic gut) are tied loosely around it with about 1 mm spacing. The length of nerve thus affected is 45 mm long. The ligatures are tied such that the diameter of the nerve is seen to be just barely constricted when viewed with 40× magnification. The desired degree of constriction retards, but does not arrest, circulation through the superficial epineurial vasculature. The incision is closed in layers. In each animal, an identical dissection is performed on the opposite side, except that the sciatic nerve is not ligated. Groups of control rats are used, wherein some rats are not operated upon and others receive bilateral sham procedures (sciatic exposure without ligation).

Each group is subsequently treated with AMDACs only, PRP only, and AMDACs in combination with PRP. The amounts of AMDACs, and the ratio of AMDACs to PRP, are varied to assess dose-dependency of the different treatments. The animals are inspected every 1 or 2 days during the first 14 postoperative days and at about weekly intervals thereafter. During these inspections, each rat is placed upon a table and carefully observed for 1-2 minutes. Notes are made of the animal's gait, the posture of the affected hind paw, the condition of the hind paw skin, and the extent, if present, of autotomy. Particular attention is given to the condition of the claws because autotomy involving frank tissue damage can be indicated by gnawed claw tips. Postoperative, post-administration behavior of the rats is observed, including appetite and hyperalgesic responses to noxious radiant heat and chemogenic pain.

Assessment of Response to Noxious Heat

The rats are placed beneath an inverted, clear plastic cage (18×28×13 cm) upon an elevated floor of window glass. A radiant heat source beneath the glass floor is aimed at the plantar hind paw. Stimulus onset activates a timer controlled by a photocell positioned to receive light reflected from the hind paw. The hind paw withdrawal reflex interrupts the photocell's light and automatically stopped the timer. Latencies are measured to the nearest 0.1 sec. The hind paws are tested alternately with 5 min intervals between consecutive tests. Five latency measurements are taken for each hind paw in each test session. The 5 latencies per side are averaged and a difference score is computed by subtracting the average latency of the control side from the average latency of the ligated side. Difference scores are compared for each treatment group, i.e., AMDACs only, PRP only, and AMDACs combined with PRP.

Assessment of Response to Noxious Pressure Stimulation

A conical stylus with a hemispherical tip (1.2 mm radius) is placed upon the middle of hind paw dorsum between the second and third or third and fourth metatarsals. The animal is restrained gently between cupped hands and calibrated pressure of gradually increasing (ca. 25.5 g/sec) intensity is applied until the rat withdraws the hind paw. The hind paws are tested alternately at 3-4 min intervals. Three measurements are taken for each side, averaged, and a difference score computed by subtracting the average of the control side from the average of the ligated side. Difference scores are compared for each treatment group, i.e., AMDACs only, PRP only, and AMDACs combined with PRP.

### EQUIVALENTS

The compositions and methods provided herein, and embodiments of the same, are not to be limited in scope by the specific embodiments described herein. Indeed, various modifications in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

What is claimed is:

1. A composition comprising amnion derived adherent cells and platelet rich plasma, wherein said composition is suitable for injection into an individual, and wherein said AMDACs are OCT-4⁻ as determinable by RT-PCR, are adherent to tissue culture plastic, and are not trophoblasts.

2. The composition of claim 1, wherein said composition does not comprise an implantable bone substitute, and does not require thrombin to retain said AMDACs at a site of injection of said composition into said individual.

3. The composition of claim 1, wherein injection of said composition to said individual results in prolonged localization of said AMDACs at the site of injection, relative to AMDACs not combined with platelet rich plasma.

4. The composition of claim 1, wherein said platelet rich plasma is autologous platelet rich plasma.

5. The composition of claim 1, wherein said platelet rich plasma is derived from placental perfusate.

6. The composition of claim 1, wherein the volume to volume ratio of AMDACs to platelet rich plasma in the composition is between about 10:1 and 1:10.

7. The composition of claim 1, wherein the volume to volume ratio of AMDACs to platelet rich plasma in the composition is about 1:1.

8. The composition of claim 1, wherein the ratio of the number of AMDACs to the number of platelets in the platelet rich plasma is between about 100:1 and 1:100.

9. The composition of claim 1, wherein the ratio of the number of AMDACs to the number of platelets in the platelet rich plasma is about 1:1.

10. A method of transplantation comprising administering the composition of claim 1 by injection, wherein said injection results in prolonged localization of said AMDACs at the site of injection, as compared to injection of AMDACs not combined with platelet rich plasma, wherein said AMDACs are OCT-4⁻ as determinable by RT-PCR, are adherent to tissue culture plastic, and are not trophoblasts.

11. The method of claim 10, wherein said composition does not comprise an implantable bone substitute, and does not require thrombin to retain said AMDACs at a site of said injection of said composition into said individual.

12. The method of claim 10, wherein said platelet rich plasma is autologous platelet rich plasma.

13. The method of claim 10, wherein said platelet rich plasma is derived from placental perfusate.

14. The method of claim 10, wherein said AMDACs and said platelet rich plasma are combined to form said composition ex vivo prior to said injecting the individual.

15. The method of claim 10, wherein said platelet rich plasma is injected into the individual in a first step, and AMDACs are injected into or near the site of platelet rich plasma injection in a second step, and said composition is formed in vivo.

16. The method of claim 10, wherein transplantation of said composition comprising AMDACs and platelet rich plasma prolongs localization of the AMDACs at the site of injection or implantation for 1, 2, 3, 4, 5, 6, 7, 8, 9, 10, 11, 12, 13, 14, 15, 16, 17, 18, 19, 20 or 21 days or more, post-transplant, as compared to AMDACs not combined with platelet rich plasma.

17. The method of claim 10, wherein the volume to volume ratio of AMDACs to platelet rich plasma in the composition is between about 10:1 and 1:10.

18. The method of claim 10, wherein the volume to volume ratio of AMDACs to platelet rich plasma in the composition is about 1:1.

19. The method of claim 10, wherein the ratio of the number of AMDACs to the number of platelets in the platelet rich plasma is between about 100:1 and 1:100.

20. The method of claim 10, wherein the ratio of the number of AMDACs to the number of platelets in the platelet rich plasma is about 1:1.

21. A method of treating an individual having or critical limb ischemia, comprising administering to the individual a therapeutically effective amount of a composition comprising AMDACs and platelet rich plasma, wherein said AMDACs are OCT-4⁻ as determinable by RT-PCR, are adherent to tissue culture plastic, and are not trophoblasts.

22. A method of treating an individual having leg ulcer, comprising contacting the leg ulcer with a therapeutically effective amount of a composition comprising AMDACs and platelet rich plasma, wherein said AMDACs are OCT-4⁻ as determinable by RT-PCR, are adherent to tissue culture plastic, and are not trophoblasts.

23. The method of claim 22, wherein the leg ulcer is a venous leg ulcer, arterial leg ulcer, diabetic leg ulcer, decubitus ulcer, or split thickness skin grafted ulcer.

24. A method of treating an individual having degenerative disc disorder, comprising administering to the individual a therapeutically effective amount of a composition comprising AMDACs and platelet rich plasma, wherein said AMDACs are OCT-4⁻ as determinable by RT-PCR, are adherent to tissue culture plastic, and are not trophoblasts.

25. A method of treating an individual having herniated disc, comprising contacting the herniated disc with a therapeutically effective amount of a composition comprising AMDACs and platelet rich plasma, wherein said AMDACs are OCT-4⁻ as determinable by RT-PCR, are adherent to tissue culture plastic, and are not trophoblasts.

26. A method of treating an individual having neuropathic pain, comprising administering to the individual a therapeutically effective amount of a composition comprising AMDACs and platelet rich plasma, wherein said AMDACs are OCT-4⁻ as determinable by RT-PCR, are adherent to tissue culture plastic, and are not trophoblasts.

27. A method of treating an individual having or at risk of developing a disease or disorder associated with or caused by an inappropriate or unwanted immune response, comprising administering to the individual a therapeutically effective amount of amnion-derived adherent cells (AMDACs), or culture medium conditioned by AMDACs, wherein said therapeutically effective amount is an amount sufficient to cause a detectable improvement in one or more symptoms of said disease, disorder or condition, and wherein said AMDACs are OCT-4⁻ as determinable by RT-PCR, and are adherent to tissue culture plastic.

28. The method of claim 27, wherein said AMDACs are OCT-4⁻ as determinable by RT-PCR, and CD49f⁺, CD105⁺, and CD200⁺ as determinable by flow cytometry.

29. The method of claim 27, wherein said AMDACs are positive for VEGFR1/Flt-1 (vascular endothelial growth factor receptor 1) and VEGFR2/KDR (vascular endothelial growth factor receptor 2), as determinable by immunolocalization.

30. The method of claim 27, wherein said AMDACs are CD90⁺ and CD117⁻ as determinable by flow cytometry, and HLA-G-, as determinable by RT-PCR.

31. The method of claim 30, wherein said AMDACs are OCT-4⁻ and HLA-G⁻, as determined by RT-PCR, and CD49f⁺, CD90⁺, CD105⁺, and CD117⁻ as determinable by flow cytometry.

32. The method of claim 27, wherein said AMDACs are additionally one or more of CD9⁺, CD10⁺, CD44⁺, CD54⁺, CD98⁺, Tie-2⁺ (angiopoietin receptor), TEM-7⁺ (tumor endothelial marker 7), CD31⁻, CD34⁻, CD45⁻, CD133⁻, CD143⁻, CD146⁻, or CXCR4⁻ (chemokine (C-X-C motif) receptor 4) as determinable by immunolocalization.

33. The method of claim 27, wherein said AMDACs are additionally CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and CXCR4⁻ as determinable by immunolocalization.

34. The method of claim 27, wherein said AMDACs are OCT-4⁻, as determinable by RT-PCR, and CD49f⁺, HLA-G⁻, CD90⁺, CD105⁺, CD117⁻, and CD200⁺, as determinable by immunolocalization, and wherein said AMDACs:
(a) express one or more of CD9, CD10, CD44, CD54, CD98, CD200, Tie-2, TEM-7, VEGFR1/Flt-1, or VEGFR2/KDR (CD309), as determinable by immunolocalization;
(b) lack expression of CD31, CD34, CD38, CD45, CD133, CD143, CD144, CD146, CD271, CXCR4, HLA-G, or VE-cadherin, as determinable by immunolocalization;
(c) lack expression of SOX2, as determinable by RT-PCR;
(d) express mRNA for ACTA2, ADAMTS1, AMOT, ANG, ANGPT1, ANGPT2, ANGPTL1, ANGPTL2, ANGPTL4, BAH, CD44, CD200, CEACAM1, CHGA, COL15A1, COL18A1, COL4A1, COL4A2, COL4A3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, GRN, HGF, HEY1, HSPG2, IFNB1, IL8, IL12A, ITGA4, ITGAV, ITGB3, MDK, MMP2, MYOZ2, NRP1, NRP2, PDGFB, PDGFRA, PDGFRB, PECAM1, PF4, PGK1, PROX1, PTN, SEMA3F, SERPINB5, SERPINC1, SERPINF1, TIMP2, TIMP3, TGFA, TGFB1, THBS1, THBS2, TIE1, TIE2/TEK, TNF, TNNI1, TNFSF15, VASH1, VEGF, VEGFB, VEGFC, VEGFR1/FLT1, or VEGFR2/KDR;
(e) produce one or more of the proteins CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type IIB precursor, Wnt-9 protein, glial fibrillary acidic protein, astrocyte, myosin-binding protein C, or myosin heavy chain, nonmuscle type A;
(f) secrete vascular endothelial growth factor (VEGF), hepatocyte growth factor (HGF), interleukin-8 (IL-8), monocyte chemotactic protein-3 (MCP-3), FGF2, Follistatin, G-CSF, EGF, ENA-78, GRO, IL-6, MCP-1, PDGF-BB, TIMP-2, uPAR, or galectin-1 into culture medium in which the AMDACs grows;
(g) express micro RNAs miR-17-3p, miR-18a, miR-18b, miR-19b, miR-92, or miR-296 at a higher level than an equivalent number of bone marrow-derived mesenchymal stem cells;
(h) express micro RNAs miR-20a, miR-20b, miR-221, miR-222, miR-15b, or miR-16 at a lower level than an equivalent number of bone marrow-derived mesenchymal stem cells;
(i) express miRNAs miR-17-3p, miR-18a, miR-18b, miR-19b, miR-92, miR-20a, miR-20b, miR-296, miR-221, miR-222, miR-15b, or miR-16; or
(j) express increased levels of CD202b, IL-8 or VEGF when cultured in less than about 5% O₂, compared to expression of CD202b, IL-8 or VEGF when cultured under 21% O₂.

35. The method of claim 34, wherein said AMDACs are OCT-4⁻, as determined by RT-PCR, and CD49f⁺, HLA-G⁻, CD90⁺, CD105⁺, and CD117⁻, as determined by immunolocalization, and wherein said AMDACs:
(a) express CD9, CD10, CD44, CD54, CD98, CD200, Tie-2, TEM-7, VEGFR1/Flt-1, and VEGFR2/KDR (CD309), as determinable by immunolocalization;
(b) lack expression of CD31, CD34, CD38, CD45, CD133, CD143, CD144, CD146, CD271, CXCR4, HLA-G, and VE-cadherin, as determinable by immunolocalization;
(c) lack expression of SOX2, as determinable by RT-PCR;
(d) express mRNA for ACTA2, ADAMTS1, AMOT, ANG, ANGPT1, ANGPT2, ANGPTL1, ANGPTL2, ANGPTL4, BAIL CD44, CD200, CEACAM1, CHGA, COL15A1, COL18A1, COL4A1, COL4A2, COL4A3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, GRN, HGF, HEY1, HSPG2, IFNB1, IL8, IL12A, ITGA4, ITGAV, ITGB3, MDK, MMP2, MYOZ2, NRP1, NRP2, PDGFB, PDGFRA, PDGFRB, PECAM1, PF4, PGK1, PROX1, PTN, SEMA3F, SERPINB5, SERPINC1, SERPINF1, TIMP2, TIMP3, TGFA, TGFB1, THBS1, THBS2, TIE1, TIE2/TEK, TNF, TNNI1, TNFSF15, VASH1, VEGF, VEGFB, VEGFC, VEGFR1/FLT1, and VEGFR2/KDR as determinable by RT-PCR;
(e) produce the proteins CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type IIB precursor, Wnt-9 protein, glial fibrillary acidic protein, astrocyte, myosin-binding protein C, and/or myosin heavy chain, nonmuscle type A;
(f) secrete VEGF, HGF, IL-8, MCP-3, FGF2, Follistatin, G-CSF, EGF, ENA-78, GRO, IL-6, MCP-1, PDGF-BB, TIMP-2, uPAR, and Galectin-1 into culture medium in which the cell grows;
(g) express micro RNAs miR-17-3p, miR-18a, miR-18b, miR-19b, miR-92, and miR-296 at a higher level than an equivalent number of bone marrow-derived mesenchymal stem cells;
(h) express micro RNAs miR-20a, miR-20b, miR-221, miR-222, miR-15b, and miR-16 at a lower level than an equivalent number of bone marrow-derived mesenchymal stem cells;
(i) express miRNAs miR-17-3p, miR-18a, miR-18b, miR-19b, miR-92, miR-20a, miR-20b, miR-296, miR-221, miR-222, miR-15b, and miR-16; or
(j) expresses increased levels of CD202b, IL-8 and/or VEGF when cultured in less than about 5% O₂, compared to expression of CD202b, IL-8 and/or VEGF under 21% O₂.

36. The method of any of claims 27-36, comprising additionally administering a second type of stem cells to said individual.

37. The method of claim 36, wherein said second type of stem cells are embryonic stem cells, stem cells isolated from peripheral blood, stem cells isolated from placental blood, stem cells isolated from placental perfusate, non-AMDAC stem cells isolated from placental tissue, stem cells isolated from umbilical cord blood, umbilical cord stem cells, bone marrow-derived mesenchymal stem cells, adipose-derived stem cells, hematopoietic stem cells, or somatic stem cells.

38. The method of any of claims 27-36 wherein said disease or disorder is an allergy, asthma, or a reaction to an antigen exogenous to said individual.

39. The method of claim 38, wherein said disease or disorder is graft-versus-host disease.

40. The method of any of claims 27-36 wherein said disease or disorder is an autoimmune disease.

41. The method of claim 40 wherein said autoimmune disease is inflammatory bowel disease, multiple sclerosis, rheumatoid arthritis, psoriasis, lupus erythematosus, diabetes, mycosis fungoides, or scleroderma.

42. The method of claim 40, wherein said autoimmune disease is one or more of Addison's disease, alopecia areata, ankylosing spondylitis, antiphospholipid antibody syndrome, antiphospholipid syndrome (primary or secondary), asthma, autoimmune gastritis, autoimmune hemolytic anemia, autoimmune hepatitis, autoimmune inner ear disease, autoimmune lymphoproliferative disease, autoimmune thrombocytopenic purpura, Balo disease, Behcet's disease, bullous pemphigoid, cardiomyopathy, celiac disease, Chagas disease, chronic inflammatory demyelinating polyneuropathy, cicatrical pemphigoid (e.g., mucous membrane pemphigoid), cold agglutinin disease, degos disease, dermatitis hepatiformis, dermatomyositis (juvenile), essential mixed cryoglobulinemia, Goodpasture's syndrome, Graves' disease, Guillain-Barre syndrome, Hashimoto's thyroiditis (Hashimoto's disease; autoimmune thyroditis), idiopathic pulmonary fibrosis, idiopathic thrombocytopenia purpura, IgA nephropathy, juvenile arthritis, lichen planus, Ménière disease, mixed connective tissue disease, morephea, myasthenia gravis, narcolepsy, neuromyotonia, pediatric autoimmune neuropsychiatric disorders (PANDAs), pemphigus vulgaris, pernicious anemia, polyarteritis nodosa, polychondritis, polymyalgia rheumatica, polymyositis (e.g., with dermatomyositis), primary agammaglobulinemia, primary biliary cirrhosis, Raynaud disease (Raynaud phenomenon), Reiter's syndrome, relapsing polychondritis, rheumatic fever, Sjogren's syndrome, stiff-person syndrome (Moersch-Woltmann syndrome), Takayasu's arteritis, temporal arteritis (giant cell arteritis), uveitis, vasculitis (e.g., vasculitis not associated with lupus erythematosus), vitiligo, and/or Wegener's granulomatosis.

43. The method of claim 42, wherein said inflammatory bowel disease is Crohn's disease.

44. The method of claim 43, wherein said Crohn's disease is gastroduodenal Crohn's disease, jejunoileitis, ileocolitis, or Crohn's colitis.

45. The method of claim 44, wherein said inflammatory bowel disease is ulcerative colitis.

46. The method of claim 45, wherein said ulcerative colitis is pancolitis, limited colitis, distal colitis, or proctitis.

47. The method of claim 45, wherein said symptom is one or more of inflammation and swelling of a part of the GI tract, abdominal pain, frequent emptying of the bowel, diarrhea, rectal bleeding, anemia, weight loss, arthritis, skin problems, fever, thickening of the intestinal wall, formation of scar tissue in the intestine of the individual, formation of sores or ulcers in the intestine of the individual, development of one or more fistulas in the wall of the intestinal of the individual, development of one or more fissures in the anus of the individual, development of nutritional deficiencies (e.g., deficiencies in one or more of proteins, calories, vitamins), development of kidney stones, or development of gallstones.

48. The method of claim 45, wherein said symptom is one or more of abdominal pain, bloody diarrhea, fevers, nausea, abdominal cramps, anemia, fatigue, weight loss, loss of appetite, rectal bleeding, loss of bodily fluids and nutrients, skin lesions, joint pain, growth failure, osteoporosis, eye inflammation, or liver disease.

49. The method of claim 41, wherein said disease or disorder is scleroderma.

50. The method of claim 49, wherein the scleroderma is diffuse scleroderma, limited scleroderma (CREST syndrome), morphea, or linear scleroderma.

51. The method of claim 49, wherein said symptoms comprise one or more of hardening of the skin of the face, hardening of the skin of the fingers, Reynaud's syndrome, inappropriate vasoconstriction in an extremity, calcinosis, telangiectasia, or esophageal dysmotility.

52. The method of claim 41, wherein said disease or disorder is psoriasis.

53. The method of claim 52, wherein said symptom of psoriasis is psoriatic arthritis.

54. The method of claim 52, wherein said symptom of psoriasis is one or more of scaling of the skin, redness of the skin, thickening of the skin, formation of plaques, discoloration under the nail plate, pitting of the nails, lines going across the nails, thickening of the skin under the nail, onycholysis, development of pustules, joint or connective tissue inflammation, inflammation of the skin, or exfoliation of the skin.

55. The method of claim 41, wherein said disease or disorder is multiple sclerosis.

56. The method of claim 55, wherein said symptom is one or more of a sensory disturbance in a limb of the individual, optic nerve dysfunction, pyramidal tract dysfunction, bladder dysfunction, bowel dysfunction, sexual dysfunction, ataxia, or diplopia.

57. The method of claim 41, wherein said disease or disorder is rheumatoid arthritis.

58. The method of claim 57, wherein said rheumatoid arthritis involves one or more of pyoderma gangrenosum, neutrophilic dermatosis, Sweet's syndrome, viral infection, erythema nodosum, lobular panniculitis, atrophy of digital skin, palmar erythema, diffuse thinning (rice paper skin), skin fragility, subcutaneous nodules on an exterior surface, e.g., on the elbows, fibrosis of the lungs (e.g., as a consequence of methotrexate therapy), Caplan's nodules, vasculitic disorders, nail fold infarcts, neuropathy, nephropathy, amyloidosis, muscular pseudohypertrophy, endocarditis, left ventricular failure, valulitis, scleromalacia, mononeuritis multiplex, or atlanto-axial subluxation.

59. The method of claim 41, wherein said disease or disorder is lupus erythematosus.

60. The method of claim 59, wherein said symptom of lupus erythematosus is one or more of malar rash, development of thick red scaly patches on the skin, alopecia, mouth ulcers, nasal ulcers, vaginal ulcers, skin lesions, joint pain, anemia deficiency, iron deficiency, lower than normal platelet and white blood cell counts, antiphospholipid antibody syndrome, presence of anticardiolipin antibody in the blood, pericarditis, myocarditis, endocarditis, lung inflammation, pleural inflammation, pleuritis, pleural effusion, lupus pneumonitis, pulmonary hypertension, pulmonary emboli, pulmonary hemorrhage, autoimmune hepatitis, jaundice, presence of antinuclear antibody (ANA) in the blood, presence of smooth muscle antibody (SMA) in the blood, presence of liver/kidney microsomal antibody (LKM-1) in the blood, presence of anti-mitochondrial antibody (AMA) in the blood, hematuria, proteinuria, lupus nephritis, renal failure, development of membranous glomerulonephritis with “wire loop” abnormalities, seizures, psychosis, abnormalities in the cerebrospinal fluid, deficiency in CD45 phosphatase and/or increased expression of CD40 ligand in T cells of the individual, lupus gastroenteritis, lupus pancreatitis, lupus cystitis, autoimmune inner ear disease, parasympathetic dysfunction, retinal vasculitis, systemic vasculitis, increased expression of FcεRIγ, increased and sustained calcium levels in T cells, increase of inositol triphosphate in the blood, reduction in protein kinase C phosphorylation, reduction in Ras-MAP kinase signaling, or a deficiency in protein kinase A I activity.

61. The method of claim 41, wherein said disease or disorder is diabetes.

62. The method of claim 61 wherein said symptom is one or more of abnormally high blood sugar, lack of insulin resistance as determined by a glucose tolerance test, fatigue, or loss of consciousness.

63. The method of claim 41, wherein said disease, disorder or condition is mycosis fungoides (Alibert-Bazin syndrome).

64. The method of claim 63, wherein said mycosis fungoides is in the patch phase.

65. The method of claim 63, wherein said mycosis fungoides is in the skin tumor phase.

66. The method of claim 63 herein said mycosis fungoides is in the skin redness (erythroderma) stage.

67. The method of claim 63, wherein said mycosis fungoides is in the lymph node stage.

68. The method of claim 63, wherein said symptom is one or more of development of flat red patches that are itchy, development of flat, red patches that are raised and hard (plaques), development of raised lumps (nodules), development of large red itchy scaly areas over the body, cracking of the skin of the palms and soles, thickening of the skin of the palms and soles, or inflammation of the lymph nodes.
